SEQUENCE LISTING (94 sequences)

SEQ ID NO: 1 - protein, 256 aa, Sorghum halepense
MAAVLAALVTGGSCAPKKFPPGPNITTNYNGQWLSARATWYGQPNGAGPDDNGGACGIKNVNLPPYNGFTACGNVPIFKDGKGCGSCYEVRCKEMPECSGNPITVFITDMNYEPIAPYHFDFSGKAFGSLAKPGLNDKLRHCGIMNVEFRRVRCKLGGKIMFHVEKGSNPNYLAVLVKNVADDGNIVLMELEDKASPGFKPMKQSWGAVWRFDTPKPVKGPFSIRLTSESGKKLVAPNVIPATWKPDTLYNSNIQF

SEQ ID NO: 2 - protein, 242 aa, Sorghum halepense
APKKFPPGPNITTNYNGQWLSARATWYGQPNGAGPDDNGGACGIKNVNLPPYNGFTACGNVPIFKDGKGCGSCYEVRCKEMPECSGNPITVFITDMNYEPIAPYHFDFSGKAFGSLAKPGLNDKLRHCGIMNVEFRRVRCKLGGKIMFHVEKGSNPNYLAVLVKNVADDGNIVLMELEDKASPGFKPMKQSWGAVWRFDTPKPVKGPFSIRLTSESGKKLVAPNVIPATWKPDTLYNSNIQF

SEQ ID NO: 3 - protein, 422 aa, Sorghum halepense
MALGSNAMRVFFLLAMVVCAAHAAGKAAPKEKEKGKDDKSGGAPAEAPSGSAGGSDISKLGAKGDGKTDSTKALNEAWAAACGKEGPQTLMIPKGDYLTGPLNFSGPCKGSVTIQLDGNLLGTTDLSAYKTNWIEIEHVDNLVISGKGTLDGQGKQVWDNNKCAQKYDCKILPNSLVLDYVNNGEVSGITLLNAKFFHMNVFQCKGVTIKDVTVTAPGDSPNTDGIHIGDSSKVTITGTTIGVGDDCISIGPGSTGINVTGVTCGPGHGISVGSLGRYKDEKDVTDINVKDCTLKKTSNGVRIKSYEDAACVITASKLHYENIAMDDVANPIIIDMKYCPNKICTAKGDSKVTVKDVTFKNITGTSSTPEAVSLLCSDKIPCSGVTMDNIKVEYKGTNNKTMAVCQNAKGSATGCLKELACF

SEQ ID NO: 4 - protein, 399 aa, Sorghum halepense
AGKAAPKEKEKGKDDKSGGAPAEAPSGSAGGSDISKLGAKGDGKTDSTKALNEAWAAACGKEGPQTLMIPKGDYLTGPLNFSGPCKGSVTIQLDGNLLGTTDLSAYKTNWIEIEHVDNLVISGKGTLDGQGKQVWDNNKCAQKYDCKILPNSLVLDYVNNGEVSGITLLNAKFFHMNVFQCKGVTIKDVTVTAPGDSPNTDGIHIGDSSKVTITGTTIGVGDDCISIGPGSTGINVTGVTCGPGHGISVGSLGRYKDEKDVTDINVKDCTLKKTSNGVRIKSYEDAACVITASKLHYENIAMDDVANPIIIDMKYCPNKICTAKGDSKVTVKDVTFKNITGTSSTPEAVSLLCSDKIPCSGVTMDNIKVEYKGTNNKTMAVCQNAKGSATGCLKELACF

SEQ ID NO: 5 - protein, 410 aa, Sorghum halepense
MACTGNAMRAFFLLAFVCAAHAGKDAPAKDGDAKAASGPGGSFDISKLGASGDGKKDSTKAVQEAWTSACGGTGKQTILIPKGDYLVGPLNFTGPCKGDVTIQVDGNLLATTDLSQYKGNWIEILRVDNLVITGKGKLDGQGPAVWSKNSCAKKYDCKILPNSLVLDYVNNGEVSGITLLNAKFFHMNVFQCKGVTIKDVTVTAPGDSPNTDGIHIGDSSKVTITGTTIGVGDDCISIGPGSTGINVTGVTCGPGHGISVGSLGRYKDEKDVTDINVKDCTLKKTSNGVRIKSYEDAACVITASKLHYENIAMDDVANPIIIDMKYCPNKICTAKGDSKVTVKDVTFKNITGTSSTPEAVSLLCSDKIPCSGVTMDNIKVEYKGTNNKTMAVCQNAKGSATGCLKELACF

SEQ ID NO: 6 - protein, 388 aa, Sorghum halepense
GKDAPAKDGDAKAASGPGGSFDISKLGASGDGKKDSTKAVQEAWTSACGGTGKQTILIPKGDYLVGPLNFTGPCKGDVTIQVDGNLLATTDLSQYKGNWIEILRVDNLVITGKGKLDGQGPAVWSKNSCAKKYDCKILPNSLVLDYVNNGEVSGITLLNAKFFHMNVFQCKGVTIKDVTVTAPGDSPNTDGIHIGDSSKVTITGTTIGVGDDCISIGPGSTGINVTGVTCGPGHGISVGSLGRYKDEKDVTDINVKDCTLKKTSNGVRIKSYEDAACVITASKLHYENIAMDDVANPIIIDMKYCPNKICTAKGDSKVTVKDVTFKNITGTSSTPEAVSLLCSDKIPCSGVTMDNIKVEYKGTNNKTMAVCQNAKGSATGCLKELACF

SEQ ID NO: 7 - protein, 261 aa, Sorghum halepense
MPRGGKPAASSKPNPFDSDSDSESNNKPAAKKSGAYQAPADAKKRYKDGFRDAGGLENQSVEELQHYAAYKAEETTDALEGCLRIAEDIKKDASDTLVTLHKQGEQISRTHEKAVEIDQDLSKSESLLGSLGGFFSKPWKPKKTKQIKGPAHVSRDDSFKKKASRMEQRDKLGLSPRGKRDPRHYAEATDAMDKVQIEKKKQDDALDDLSGVLGQLKGMAVDMGSELDRQNEALDNLQGDVDELNSRVKGANQRARKLVAK

SEQ ID NO: 8 - protein, 118 aa, Sorghum halepense
MASEEGVVIACHTKAEFDAQMAKAKEAGKLVVIDFTASWCGPCRAIAPLFVEHAKKYTQAVFLKVDVDELKEVTAEYKIEAMPTFHFIKNGETVETIVGARKDELLALIQKHTASASA

SEQ ID NO: 9 - protein, 149 aa, Sorghum halepense
MADQLTDDQIAEFKEAFSLFDKDGDGCITTKELGTVMRSLGQNPTEAELQDMINEVDADGNGTIDFPEFLNLMARKMKDTDSEEELKEAFRVFDKDQNGFISAAELRHVMTNLGEKLTDEEVDEMIREADVDGDGQINYEEFVKVMMAK

SEQ ID NO: 10 - protein, 610 aa, Sorghum halepense
RRVDVVPGGAGSPRSTSSISRGPDAGVSEKTSGAWSGGGRLRSDGAGGNAFPWSNAMLQWQRTGFHFQPHMNWMNDPNGPVYYKGWYHLFYQYNPDGAIWGNKIAWGHAVSRDLIHWRHLPLAMVPDQWYDTNGVWTGSATTLPDGRLAMLYTGSTNASVQVQCLAVPADDADPLLTNWTKYEGNPVLYPPPGIGPKDFRDPTTAWFDPSDNTWRIVIGSKDDAEGDHAGIAVVYRTKDFVSFELLPGLLHRVARTGMWECIDFYPVATRGKASGNGVDMSDAFGKNGAIVGDVVHVMKASMDDDRHDYYALGRYDAATNEWTPLDAEKDVGIGLRYDWGKFYASKTFYDPAKRRRVLWGWVGETDSERADVSKGWASLQGIPRTVLLDTKTGSNLLQWPVEEAETLRTNSTDLSGITIDYGSAFPLNLRRATQLDIEAEFQLDRRAVMSLNEADVGYNCSTSGGAAARGALGPFGLLVLADKHLREQTAVYFYVAKGLDGSLTTHFCQDESRSSSANDIVKRVVGSSVPVLDDETTLSLRVLVDHSIVESFAQGGRSTATSRVYPTEAIYANAGVFLFNNATAARVTAKKLVVHEMDSSYNHDYMVTDI

SEQ ID NO: 11 - protein, 173 aa, Sorghum halepense
MASILVTTTTATAILLCVLFCAAAANTTVANDPNLPDYVIQGRVYCDTCRAGFVTNVTEYMAGAKVRLECKHFGTGEVERAIDGVTDATGTYTIELKDSHEEDICQVVLVQSPRKDCDQTQPLRDRAGVLLTRNVGIADSLRPANPLGYFKDVPLPVCAALLKQLDSDNDDDQ

SEQ ID NO: 12 - protein, 572 aa, Sorghum halepense
LHCTGTAMVRASHTVYPELQSLEVEKVDEMSRTGYHFQPPKHWINDPNGPMYYKGLYHLFYQYNPKGAVWGNIEWAHSVSTDLIDWTALDPGIYPSKNFDIKGCWSGSATVLPSGMPIVMYTGIDPNDHQVQNLAYPKNLSDPFLREWVKPDYNPIISPDSGINASAFRDPTTAWLGPDKHWRLLVGSRVDDKGLAVLYRSRDFKRWVKAHHPLHSGLTGMWECPDFFPVAVHGGSRHHRRGVDTAELHDRALAEEVKYVLKVSLDMTRYEYYTVGSYDHATDRYTPDAGFRDNDYGLRYDYGDFYASKSFYDPAKRRRILWGWANESDTVPDDRRKGWAGIQAIPRKLWLSPGGKQLIQWPVEEVKALRGKHVNVSDQVVKGGQYFEVDGFKSVQSDVEVTFAVDDLSKAEQFNPKWFTDPQRLCKKRGAREKGEVGPFGLWVLAAGDLTERTAVFFRVFRTNTSRLVVLMCNDPTNSTFEAQVYRPTFASFVNHDIAKTKTIALRTLIDHSVVESFGAGGRTCILSRVYPKKALGDNAHLFVFNHGEVDVKVAKLDAWEMRTPKMNAPAQ

SEQ ID NO: 13 - protein, 136 aa, Sorghum halepense
KVIDVVVSAAPPDKQKETLHAAQKHLKPLTSALDKAKETGDEKEIARLVLSVEITLAMTKNAPPEKKLKTMEDSINSVAAPSPLDCPTVDKAYCEMHAKIQKAVNGFATADLANKMSEAQATVLEETLYTAGSTIN

SEQ ID NO: 14 - protein, 166 aa, Sorghum halepense
MTQQSMVALVAAGVLLLAGVASAEKAGGFVVTGRVYCAPCRAGFETNVSKSVAGATVEVVCRHFEASKETLKAEATTDDFGWYKLEIDQDHQEEICEVVLKKSPDPACAEIEEQRARARVPLTSNNGIKQKGTRYANPIAFFRKDPLKECGAILQKYDLKDASDTP

SEQ ID NO: 15 - protein, 426 aa, Sorghum halepense
RAPAPARSLHPIPGLQHEIHTPMAFISNDVVAMKAAAVSALLVFAAVAGGAPSMPAGPLDIAQLGAKGDGKSDSTPMILKAWKNACDATGVQKIVIPPGNYLTGGLELKGPCKSSIIIRLDGNLLGTGDLNAYKRNWIEIENVDNLSINGHGTIDGQGSLVWNKNDCQHSYNCKVLPNSLVLDFVTNAQIRGITLANSKFFHLNIFASKNVLIDKVTVKAPGNSPNTDGIHMGDSENVTISGTTIGVGDDCISIGPGSKTIRIDGVKCGPGHGISVGSLGRYKDEKDVEDVKVKGCTLVGTTNGLRIKSYEDSKSSPKVTKFVYEDVTMDNVSYPIIIDQKYCPNNICVRSGASKVAVTDVVFKNIHGTSNTPEAITLNCADNLPCQGVQLHNVDIKYNKSNNKTMAVCKNAVGKSFGLSKELACI

SEQ ID NO: 16 - protein, 446 aa, Sorghum halepense
MAVTITWVKARQIFDSRGNPTVEVDIGLSDGSYARGAVPSGASTGIYEALELRDGGSDYLGKGVLKAVSNVNTIIGPAIVGKDPTEQVEIDNFMVQQLDGTSNEWGWCKQKLGANAILAVSLAVCKAGAMVKKIPLYQHIANLAGNKTLVLPVPAFNVINGGSHAGNKLAMQEFMILPTGASSFKEAMKMGVEVYHNLKSIIKKKYGQDATNVGDEGGFAPNIQENKEGLELLKAAIEKAGYTGKVVIGMDVAASEFFSEKDKTYDLNFKEENNDGSNKISGDSLKDLYKSFVSEYPIVSIEDPFDQDDWTTYAKLTDEIGQQVQIVGDDLLVTNPTRVAKAINEKTCNALLLKVNQIGSVTESIEAVRMSKRAGWGVMASHRSGETEDTFIADLSVGLATGQIKTGAPCRSERLAKYNQLLRIEEELGDAAVYAGAKFRAPVEPY

SEQ ID NO: 17 - protein, 246 aa, Sorghum halepense
MTCKMRRRHATTTTMIAAVLCLLLFSGRLAAAEKSFGGGGYSGLEAGGQQPETGGASEAALAGAAETTTTPAAYSSGGDAAAASGGGGGGGYGGKLDPDGDPEVGLNEKAIKEIVDEHNMFRAKENAGLPPLVWNETLAKWSQKYAETLKGNCQQIHSTSPYGENLMEGTPGLTWKITVDGWSEEKKNYHFDSDTCDAGKMCGHYKAVVWKTTTSVGCGRIKCNSGDTIIMCSYWPPGNYDGVKPY

SEQ ID NO: 18 - protein, 251 aa, Sorghum halepense
MAGRKILLLLLLCAMDRVAVVVLAVAGQQGADPRALPAEWATAIKYKATMDVKTRQAFDGVVAAAPAEKRSEAVEAVLQQQLNMDVSLGKATASGDENNFVSVAGAYEKAADAVIAASPANKLGTMAFAYNGVVAPDPGRCTAAAAADKPFCETYAKTEKAFAGVIATGDSPRTRLGFTDVVLKQRLATDAAINKAYAEGDKDKIAKILAAYAQAADAVAAAAPPEKLRVMEQTFSAVAAAAHQPAAAAKA

SEQ ID NO: 19 - protein, 391 aa, Sorghum halepense
MELRPWLLLVVVAIVVSISADGGTVINVKNYGAHGNGANDDSKPLMAAWKAACGSAGAVTMVVTPGTYYIGPVQFHGPCKASSLTFQLQGSTLKAATDLSKFGNDWIEFGWVNGLTVTGGTIDGQGAASWPFNKCPVRKDCKVLPTSVLFVNNQNTVVRDVTSVNPKFFHMALLTVKNIRMSGLKISAPSNSPNTDGIHIERSSGIQITDTRISTGDDCISIGQGNDNVQIARVQCGPGHGMSVGSLGRYASEGDVTRVHVRDMTFTGTTNGVRIKTWENSPSKSHAAHMVFENMVMKDVQNPIIIDQKYCPYYNCEHKYVSGVTIQDIQFKNIKGTAATQVAVLLRCGVPCQGLVLQDVDLRYKGQGGTLAKCENAKAKYVGNQFPKPCP

SEQ ID NO: 20 - protein, 118 aa, Sorghum halepense
EIEGHHLTSAAIAGHDGAVWAQSATFPEFKPEDMTNIMKDFDEPGHLAPTGLFLGATKYMVIQGEPGAVIRGKKGSGGITVKKTGQALIIGIYDEPMTPGQCNMVVERLGDYLVEQGM

SEQ ID NO: 21 - protein, 81 aa, Sorghum halepense
RRPKPSCHEVMVGEYRPTAADAAANRTAGFGLVTNIVNGGLECNRTDDARVNNRIGFYQRYCQIFNVDAGANLDCAHQQPY

SEQ ID NO: 22 - protein, 346 aa, Sorghum halepense
LFLARGAVVRATQDTSSWPLIEPLPSYGRGRELPGGRYISLIHGSGLQDVVITGENGTIDGQGTPWWDMWKKGTLLYTRPHLLELMSSSHIIVSNVVFQDSPFWNIHPVYCSNVVIRNVTILAPHDSPNTDGIDPDSSSNICIEDCYISTGDDAIAIKSGWDEYGIAYGRPSSDITVRRITGSSPFAGFAVGSETSGGVENVLAEHLNFFSSGFGIHIKTNTGRGGFIRNVTVSDVTLDNVRYGLRIVGDVGNHPDERYNRSALPIVDALTIKNVQGQNIREAGLIKGIANSAFSRICLSNVKLTGGAPVQPWKCEAVSGGALDVQPSPCTELTSTSGTSFCTNSL

SEQ ID NO: 23 - protein, 127 aa, Sorghum halepense
MAMAKILLLILLVVVTAVVEAADPPAKWKAALTALDAMDAKMRQAVDGVAAAAPAEKQSEVQEAAMAERLDVSLALARVEETGNEKKVESMAASYEKAADLVVAAPPPDKLKVMKEAFRAVTKAAAL

SEQ ID NO: 24 - protein, 126 aa, Sorghum halepense
MATTEAAAATPVAPAEGSVIAIHSLDEWSIQIEEANSAKKLVVIDFTATWCPPCRMIAPVFAELAKKHPNVVFLKVDVDEMKTIAEQFSVEAMPTFLFMREGDVKDRVVGAAKEELANKLQLQMAQ

SEQ ID NO: 25 - protein, 131 aa, Sorghum halepense
MSWQTYVDEHLMCEIEGHHLTSAAIIGHDGTVWAQSTAFPQFKPEEMTNIMKDFDEPGFLAPTGLFLGPTKYMVIQGEPGAVIRGKKGSGGITVKKTGQALVIGIYDEPMTPGQCNMVVERLGDYLVEQGL

SEQ ID NO: 26 - protein, 413 aa, Sorghum halepense
MASASNALRVFFILAILCAVCTAKRTGAKTGDSAADSAASGASGTFDISKLGATGDGKTDSTKAVQDAWTSACRATGSATVLIPKGDYLVGPLNFVGPCKGAITIQLDGNLLGSNDLAKYKASWIELSHVDNIVMTGSGTLDGQGTAVYKKAKTGTVKAMPNTLVLFYVTNGTVSGIKLLNSKFFHINIDASKNITVKDVNITAPGDVENTDGVHVGMSTKVSITNSTIGTGDDCISVGPGSDGVMVNNIICGPGQGISIGCLGRYKDEKDVTDVTVRDCVLKKTTNGVRIKSYEDAESVLTASHLTFENIRMEEVANPIIIDQYFCPEKVCPGKKSDSSHVIVKDVTFRNITGTSSTPQAISLLCSQSQPCSGVSLIDVNVEYAGKNNKTMAVCSNAKGTAKGSVEALACLA

SEQ ID NO: 27 - protein, 273 aa, Sorghum halepense
MSGRRRRSWTIWAPLLASLLLAGLALSAKVVDEEEAEADGDDGGGASKKKKPHVNHGKFKADPWTDGHATFYGGRDGSGTTDGGACGYKGELGKDYGALTAAVGPSLYTNGAGCGACYELKGSKGTVVVTATNQAPPPVSGQKGEHFDLTMPAFLKIDEEKAGIVPITYRKVACARQGGIRYTITGNPNYNMVMVTNVGGAGDVVALSVKGNKRVKWTPMKRSWGQLWITEVNLTGESLTFRVMTGDHRKATSWHVAPRDWTYDKTYQATKNF

SEQ ID NO: 28 - protein, 442 aa, Sorghum halepense
MMMDARLRRLVFLLLLAAAAPLATAQLSQDFYKTSCPDAEKIIFGVVEKRFKADPGTAAGLLRLVFHDCFANGCDASILIDPMSNQASEKEAGPNISVKGYDVIEEIKTELEKKCPGVVSCADIVSVSARDSVKLTGGPEYSVPLGRRDSLVSNREDADNLPGPDIAVPKLIDEFSKQGFNLEEMVAMLGGGHSIGICRCFFIETDAAPIDPGYKKKISDACDGKDSGSVDMDSTSPNTFDGSYFGLVLEKKMPLTIDRLMGMDSKTEPVVQAMADKKTDFVPIFAKAMEKLSNLKVITGKDGEIRKVCSEFNNPQNSSSSSSVIRTSSVNADEVAGLSSSSSRKVGPPDAVETPAMVADEAAAKVPGGVVVSVGGDQQQPPNPEADRPGLKLRGSREPVNPAAPVEPGPGGEDAAKQQAVAALEEKKKRNMAKLRAAQAKM

SEQ ID NO: 29 - protein, 243 aa, Sorghum halepense
MAKMILLLIILVATITAAVEAAPSPAVPVPQQSSAEADKKINEVNLTLKKVFDDVIATAPPAKKQEAIDATTKQLQVAERALAKAKAGGDEKVAKLAMSYELSARIVTETPPAMKLERMEELFNAMAAPNHKTECHPNAEADKPFCETVSKLQKAFKEVRSAVAQGKKEETIDDVFLVNQEFAPTIRAINKAYADGDEKEIAAVLATYDKCADAILAAPLAEKFKVMKESIAAASRAPGKQAP

SEQ ID NO: 30 - protein, 80 aa, Sorghum halepense
MAETADMERIFKRFDTNGDGKISLSELTDALRQLGSTSADEVQRMMAEIDTDGDGCIDFNEFITFCNANPGLMKDVAKVF

SEQ ID NO: 31 - protein, 144 aa, Sorghum halepense
MMPQLRSLVALLLVATAVAAVVAVAAAGGGFVVTGRIYCDNCRAGFETNISHAIQGATVEMECRHFESQQIHDKAQATTDAGGWYKMEIAGDHQDEICDVRLLKSPEADCAEIEVNRDRCRVPLTGNDGIKQSGVRYANPIAFF

SEQ ID NO: 32 - protein, 220 aa, Sorghum halepense
PSSVVVLTPETFDSIVLDETKDVLVEFYAPWCGHCKSLAPTYEKVASVFKLDEGVVIANLDADKYRDLAEKYGVTGFPTLKFFPKGNKAGEDYDGGRDLGDFVKFINEKSGTSRDTKGQLTSEAGRIASLDVLAKEFLGASSDKRKEVLSSMEEEAAKLSGPSARHGKVYVNIAKKILEKGNEYTKKETERLDRMLEKSINPSKADEFIIKKNVLSTFSS

SEQ ID NO: 33 - protein, 121 aa, Sorghum halepense
MTSSSSFLLVVVVLAALFAVSSCDNPPAITFTIGKDSSSTKLSFATDVAISEVAVKQNGAENWSDNLKESPVKTFTLDSKDPIKGPITIRFADKDGGYHVLVDIIPADFKAGSVYKALSYV

SEQ ID NO: 34 - protein, 463 aa, Sorghum halepense
MAPGRSLRPAVAVVLWAASLVLLLAAACAGAGGASGGPGCRKHVAKVTEYGAVGDGRTLNTEAFAKAVADLSRRARDGGAALVVPPGKWLTGPFNLTSCFTLYLDEGAEILASQDMNHWPLIAPLPSYGRGRDEPGPRYINFIGGSNLTDVIITGKNGTINGQGQVWWDKFHAKELKSTRGHLLELLHSDNIIISNVTFVDAPYWNLHPTYCTNVTISGVTILAPLNSPNTDGIDPDSSTHVKIEDCYIVSGDDCVAVKSGWDEYGIKFNMPSQHIVIRRLTCISPTSAMIALGSEMSGGIRDVRAEDNIAINTESAVRIKSGAGRGGFVRDIFVRRLSLHTMKWVFWMTGNYGQHPDNTSNPNAMPEVTGINYSDVFAENVTTAGRMEGIPNDPYTGICISNVTASLAPNATELQWNCTNVKGVTSNVSPKPCPELGAEGKPCAFPVEELVIGPPALPKCSY

SEQ ID NO: 35 - protein, 176 aa, Sorghum halepense
MMGRRCITDQVLLAAVVVVAAASGGLLVAAVKDDDFFVDGSVYCDTCRAGFETNATTPIAGAKVRLECRHYMSASGAVERSAEGATDAAGRYRIELVDNRGAEEVCSVVLLSSPVPGCAEKEVGRDRAQVELVTDAGAGLATTVRRANPLGFLKSQPLPNCGEILKSYGLGSGPGY

SEQ ID NO: 36 - protein, 183 aa, Sorghum halepense
RVLTKSDHPGSHCAAPPAMAIRSSKACWISLLLALALSAVARAEEPAAEGAAEAVLTLDVDSFDEAVAKHPFMVVEFYAPWCGHCKKLAPEYETAAKELSKHDPPIVLAKVDANEEKNRPLATKYEIQGFPTLKIFRNQGKNIQEYKGPREADGIVDYLKKQVGPASKELKSQEDVATHYDDK

SEQ ID NO: 37 - protein, 148 aa, Sorghum halepense
MGGKDLTEDQIASMREAFSLFDTDGDGKIAPSELGVLMRSLGGNPTQAQLRDIAAQEKLTAPFDFPRFLELMRAHLKPEPFDRPLRDAFRVLDKDGSGTVSVADLRHVLTSIGEKLEAHEFDEWIREVDVAPDGTIRYDDFIRRIVAK

SEQ ID NO: 38 - protein, 104 aa, Sorghum halepense
ERLGASFKKAKSVLIAKIDCDEHKSLCSKYGVSGYPTIQWFPKGSLEPKKYEGQRTAEALAEFVNTEGGTNVKLATIPSSVVVLTPETFDSIVLDEAKDVLVEF

SEQ ID NO: 39 - protein, 108 aa, Sorghum halepense
WCGHCKKLAPILEEAATTLQSDEEVVIAKMDATANDVPSEFEVQGYPTMYFVTPSGKVTAYDSGRTADDIVDFIKKSKETAGATQATTTTSEKAADAAEKAEPVKDEL

SEQ ID NO: 40 - protein, 526 aa, Sorghum halepense
MATSRSLALALLLCALSSSCHAAISYPPSAMPAAAPAKGDFLACLTKSIPPRLLYARSSPAYGSIWASTVRNLKFDSDKTAKPLYIITPTEPAHIQATVACGRKHGMRVRVRSGGHDYEGLSYRSTKPETFAVVDMSLLRKVSLDGKAATAWVDSGAQLGDIYYALGKWAPKLGFPAGVCATIGVGGHFSGGGFGMMLRKHGLAVDNVVDAKVVDANGNLLDRKTMGEDYFWAIRGGGGESFGIVVSWQLKLVPVPPKVTVLQMPRSVKDGAIDLIVKWQQVAPSLPEDLMIRILAMGGTAIFEGLFLGTCKDLLPLMASRFPELGVKQGDCKEMSWVQSVAFIPMGDKATMKDLLNRTSNIRSFGKYKSDYVKDPIAKPVWEKIYAWLAKPGAGIMIMDPYGAKISAIPDRATPFPHRQGMLFNIQYVTYWSGEAAGAAPTQWSRDMYAFMEPYVTKNPRQAYVNYRDLDLGVNQVVNDISTYESGKVWGEKYFSFNFERLARIKAKVDPTDYFRNEQTIPPLFK

SEQ ID NO: 41 - protein, 199 aa, Sorghum halepense
MDSNQGVVAVVKPTLAKGTPSASFRLRNGSLNAVRLRRVFDLFDRNGDGEITVDELAQALDALGLDADRAGLAATVGAYVPDGAAGLRFEDFDKLHRALGDAFFGALADHQDDATDAGGKKGEEDEQEMREAFKVFDVDGDGFISAAELQTVLKKLGLPEASSMANVREMITNVDRDSDGRVDFSEFKCMMKGITVWGA

SEQ ID NO: 42 - protein, 150 aa, Sorghum halepense
ETTGPNMVVDMCKGVQYLNEIKDSVVAGFQWASKEGALAEENMRGICFEVCDVVLHADAIHRGGGQVIPTARRVIYASQLTAKPRLLEPVYLVEIQAPENALGGIYGVLNQKRGHVFEEMQRPGTPLYNIKAYLPVIESFGFSSQLRAAT

SEQ ID NO: 43 - protein, 206 aa, Sorghum halepense
HGALWIAVVAFLVASGSVVVIRVAEARYGPGHWNPAAPAPVATLVSEQLYNSLFLHKDDAACPAKGFYTYAAFIQAARTFPKFAATGDLSTRKREVAAFFAQISHETTGGWATAPDGQYAWGLCYKEEISPASSYCDATDKQWPCYPGKSYHGRGPIQLSWNFNYGPAGQALGFDGLRNPEVVANCSETAFRTALWFWMTPRRPKP

SEQ ID NO: 44 - protein, 266 aa, Sorghum halepense
MGVNMMSWSMQVALVVALAFLVGGAWCGPPKVAPGKNITATYGSDWLEAKATWYGKPTGAGPDDNGGACGYKDVNKAPFNSMGACGNLPIFKDGLGCGSCFEIKCDKPAECSGEAVVVHITDMNYEQIAAYHFDLAGTAFGAMAKKGEEEKLRKAGIIDMKFRRVKCKYGEKVTFHVEKGSNPNYLALLVKYVDGDGDVVGVDIKEKGGDAYQPLKHSWGAIWRKDSDKPIKFPVTVQITTEGGTKTAYEDVIPEGWKADTTYTAK

SEQ ID NO: 45 - protein, 119 aa, Sorghum halepense
MASSSSFLLAMAALAALLAVGSCSTLMTWTIGKDSTSTRLVLVASADVSEVAVKDKGATDFSDDLKESPAKTFTYESKEPIKGPLSVRFAVKGQGYRTTDDVIPADFKPGSVYKTKEQV

SEQ ID NO: 46 - protein, 121 aa, Sorghum halepense
MASSSSFCFLLAVVALAALFAIGSCGTTLTIEVGKDSTSTKLSLITNVAISEVSVKPKGATDFTDDLKESEPKTFTLDSKEPIEGPIAFRFLAKGGGYRVVDNAIPADFKAGSVYKTTEQV

SEQ ID NO: 47 - protein, 121 aa, Sorghum halepense
MASSSSFLLAMAALAALFAVGLCGNSVTLTVGKGTTPTYTHLVLVANLPIHELAIREKGAAEFLDDMKESPAKTFTQDSKAPLKGPLSVRFAVKGGGYRNRDEIFPVGLKPGAVINTNIPY

SEQ ID NO: 48 - protein, 228 aa, Sorghum halepense
MAKTTILLLLSSILVVADQAATTATQLSPAAEKSIGDLNLEVEKVIDVVVSAAPPAKQMETMHAALKHLQPIKSALAKAKESGDEKKIAKLVFRVEIAAAIINAAPADKKLMMMEDSFNSVAAPSPLDCPTVDKAYCETDSKIQKAFDGVVAAAPVEKRLEVRATILKKTMYTAGSTINKAYADGDEKKIAQVLAAYSKAADEVIAAAPADKLTIMEKTFIAAAATGN

SEQ ID NO: 49 - protein, 207 aa, Sorghum halepense
MASSSSSNHSFLLLLLFPLLLVLLLSTTVVAADSTATAEKYQKWCQVASDSPSCVKVIESIPGIQEVDYNNAGKVADLCLHFAANKTKEAKGAADTLLAAEKGKPASGCLKACATNIKSMAEVLVNLPAGQDDMNAYQTYKEVRAKFKNEKPPACEKDCWNKTSSSSSSAADIVDKFHDIWNVAKVGNMQINYIFPWPDSDDDDNIL

SEQ ID NO: 50 - DNA, 768 bp, Sorghum halepense
atggcggctgtccttgcggcgctcgtcaccggcggctcgtgcgcgcccaagaagttcccgcctggccccaacatcacaaccaactacaacggccagtggctctctgccagggccacctggtacggccagcccaacggcgccggccctgacgacaacggcggtgcgtgcggaatcaagaacgtgaacctgccaccctacaatggcttcacggcctgcggtaacgtccccatcttcaaggatggcaagggctgcggctcatgctacgaggtgagatgcaaggaaatgccggagtgttcgggcaacccgatcacggtgttcatcaccgacatgaactacgagcccatcgctccctaccacttcgacttcagcggcaaggccttcggctccctggcaaagcccgggctcaacgacaagctccgccactgcggcatcatgaacgtggagttcaggagggtgcggtgcaagcttgggggcaagatcatgttccacgttgagaaggggtccaaccccaactacctggccgtgctggtcaagaacgtggcggacgacggcaacatcgtgctcatggaactcgaggacaaggcgtcgccggggttcaagccaatgaagcaatcctggggcgccgtgtggaggtttgacacacccaagccggtcaagggccccttctccatccgcctcaccagcgagtccggcaagaagctcgtcgccccaaacgtcatcccggcgacctggaagcccgacaccctctacaactccaacatccagttc

SEQ ID NO: 51 - DNA, 1266 bp, Sorghum halepense
atggctttgggcagcaatgctatgagggtgttcttcctccttgcgatggtggtgtgcgccgcgcatgcggcggggaaggcagccccaaaggagaaagagaaggggaaggacgacaagtccggcggtgcccccgccgaggcgccctcaggctccgccgggggcagcgacatatccaagctcggagctaagggcgacggcaagacggacagcaccaaagcgctgaatgaagcgtgggcggcggcgtgcggcaaggaagggccgcagacgctcatgatccccaagggcgactacctgacagggcccctcaacttcagcgggccgtgcaagggctccgtcaccatccagctcgacggcaaccttctcggcaccacggacctgagcgcgtacaagaccaactggatcgagatcgagcacgtcgacaaccttgtcatcagcggcaaaggcaccctcgacggccagggcaaacaagtatgggacaacaacaaatgtgctcagaaatacgactgcaagatcctgcccaactcgctggtgctggactacgtgaataacggggaggtgtccgggatcacgctgctcaacgccaagttcttccacatgaacgtgttccagtgcaaaggcgtgacgatcaaggacgtgaccgtcaccgcgcccggggacagccccaacaccgacggcatccacattggtgactcctccaaggtcaccatcaccggcaccaccatcggcgtgggtgacgactgcatctccatcggccctggcagcaccgggatcaacgtcaccggcgtcacctgcgggcccggccacggcatcagcgtcggcagcctgggcaggtacaaggatgagaaggatgtgacggacatcaacgtcaaggactgcacgctcaagaagaccagcaatggcgtccggatcaagtcctacgaggacgccgcgtgcgtgatcactgcctccaagctccactacgagaacatagccatggacgacgtggccaaccccatcatcatcgacatgaagtactgccccaacaagatctgcactgccaagggtgactccaaggtcaccgtcaaggacgtcacgttcaagaacatcaccggcacctcatccaccccggaggccgtcagcctgctctgctccgacaaaatcccctgcagcggcgtcaccatggataacatcaaggtcgagtacaagggcaccaacaacaagaccatggccgtatgtcaaaacgccaagggaagcgccacaggctgcctcaaggaactggcctgcttc

SEQ ID NO: 52 - DNA, 1230 bp, Sorghum halepense
atggcgtgcacaggcaatgcgatgagagccttcttcctcctagcgttcgtctgcgccgcgcatgctgggaaggacgctcctgccaaggatggcgatgcgaaagcggcgtccgggcccggcgggtcgttcgacatcagcaagctgggcgcctccggcgacggcaagaaggacagcacgaaggctgtgcaggaggcatggacgtcggcgtgcggcggcaccgggaagcagacgatcctcatccccaagggcgactacctcgtcgggccactcaacttcaccggcccgtgcaagggcgacgtgaccatccaggtggacggcaatctgctggcgaccacggacctgagccagtacaagggtaactggatcgagatcctgcgtgtggacaacctggtgatcaccggcaaggggaagctcgacgggcagggccctgccgtgtggagcaagaactcgtgcgccaagaagtacgactgcaagatcctgcccaactcgctggtgctggactacgtgaataacggggaggtgtccgggatcacgctgctcaacgccaagttcttccacatgaacgtgttccagtgcaaaggcgtgacgatcaaggacgtgaccgtcaccgcgcccggggacagccccaacaccgacggcatccacattggtgactcctccaaggtcaccatcaccggcaccaccatcggcgtgggtgacgactgcatctccatcggccctggcagcaccgggatcaacgtcaccggcgtcacctgcgggcccggccacggcatcagcgtcggcagcctgggcaggtacaaggatgagaaggatgtgacggacatcaacgtcaaggactgcacgctcaagaagaccagcaatggcgtccggatcaagtcctacgaggacgccgcgtgcgtgatcactgcctccaagctccactacgagaacatagccatggacgacgtggccaaccccatcatcatcgacatgaagtactgccccaacaagatctgcactgccaagggtgactccaaggtcaccgtcaaggacgtcacgttcaagaacatcaccggcacctcatccaccccggaggccgtcagcctgctctgctccgacaaaatcccctgcagcggcgtcaccatggataacatcaaggtcgagtacaagggcaccaacaacaagaccatggccgtatgtcaaaacgccaagggaagcgccacaggctgcctcaaggaactggcctgcttc

SEQ ID NO: 53 - DNA, 783 bp, Sorghum halepense
atgccacgcggcggcaagcccgcggcttcgtcgaagccgaaccccttcgactccgactcggactcggagtccaataataagccggcggcgaagaagtccggggcgtaccaggcccccgccgacgccaagaagcggtacaaggacgggttccgcgacgccggcgggctggagaaccagtcggtggaggagctgcagcactacgccgcgtacaaggccgaggagaccaccgacgcgctcgagggctgcctgcgcatcgccgaggacatcaagaaggacgcgtccgacacgctcgtcaccctgcacaagcaaggggagcagatcagccgcacgcacgagaaggccgtcgagatcgaccaggacctcagcaagagcgagtcgcttctcggcagccttggcggcttcttctccaagccatggaagcccaagaaaaccaagcagatcaagggacccgcgcacgtgtcacgagatgactcgttcaagaagaaggccagccgcatggagcagagggacaagctcgggctgagcccgcgagggaagcgcgaccctcgacactacgccgaggccaccgatgccatggacaaagttcagatcgagaagaagaagcaggacgacgccctcgatgacctcagcggcgtgctgggccagctcaagggcatggccgtcgacatgggcagtgagcttgacaggcaaaacgaagcgctggataatctgcaaggcgacgtggacgagctcaactcaagggtgaagggagccaaccaacgtgcgcgcaagctggtcgccaag

SEQ ID NO: 54 - DNA, 354 bp, Sorghum halepense
atggcgtccgaggagggagtcgtgatcgcgtgccacaccaaggccgagttcgacgcccagatggccaaggccaaggaggccggcaagctggtggtcattgacttcaccgcctcctggtgtggtccttgccgcgccatcgctccactgtttgtcgagcacgccaagaagtacactcaagctgtcttcctgaaggtggacgtggacgaactgaaggaagttactgcagagtacaagatcgaggcgatgccgaccttccacttcatcaagaacggcgagacggtggagactatcgtcggtgccaggaaggacgagctcctggccctgatccagaagcataccgcgtccgcgtccgcg

SEQ ID NO: 55 - DNA, 447 bp, Sorghum halepense
atggcggaccagctcaccgacgaccagatcgccgagttcaaggaggccttcagcctcttcgacaaggacggcgatggttgcatcacaaccaaggagcttggaactgtcatgcgttcactgggtcaaaacccaaccgaggctgagcttcaggacatgatcaatgaggtcgatgccgatggcaatggcaccattgactttcctgagttcctcaacctcatggcccgcaagatgaaggacaccgactccgaggaggagctcaaggaggcgttcagggtgttcgacaaggaccagaacggcttcatctctgcagcggagctccgccatgtgatgaccaaccttggcgagaagctgaccgacgaggaggtcgatgagatgatccgcgaggctgacgtcgatggcgatggccagatcaactacgaggagttcgtgaaggtgatgatggccaag

SEQ ID NO: 56 - DNA, 1830 bp, Sorghum halepense
cgccgcgtcgacgtcgtccccggcggcgcggggtcgccgaggagcaccagcagcatcagcaggggccccgatgccggcgtgtcggagaagacgtccggcgcgtggagcggcggcggcaggctgcggagtgacggcgccggcgggaacgcgttcccgtggagcaatgcgatgctgcagtggcagcgcacgggattccacttccagccacacatgaactggatgaacgatcccaatggcccggtgtactacaagggatggtatcatctgttctaccagtacaacccagacggtgccatctggggcaacaagatcgcgtggggccacgccgtctcccgcgacctgatccactggcgccacctcccgctagccatggtgccggaccagtggtacgacaccaacggcgtctggacaggctccgccaccacgctccccgacggccgcctcgccatgctctacaccggctccaccaacgcctccgtgcaggtgcagtgcctcgccgtccccgccgacgacgccgacccgctgctcaccaactggaccaagtacgagggcaacccggtcctgtacccgccgccggggatcgggcccaaggacttccgtgaccccaccacggcgtggttcgacccgtcggacaacacctggcgcatcgtcatcgggtccaaggacgacgccgagggcgaccacgccggcatcgccgtcgtctaccggaccaaggacttcgtcagcttcgagctcctccccggcctcctccaccgcgtcgcgaggacggggatgtgggagtgcatcgacttctaccccgtcgccacccgcggcaaggcgtccgggaacggcgtcgacatgtccgacgccttcggcaagaacggcgccattgttggggacgtcgtgcacgttatgaaggccagcatggacgacgaccgccatgactactacgcgctcgggaggtacgatgcggccaccaacgagtggacgccgctcgacgccgagaaggacgtcggcatcgggctccggtatgactggggcaagttttacgcgtccaagaccttctatgaccccgccaagcgccgccgcgtgctctggggatgggtcggcgagaccgactcggagcgcgctgacgtctccaagggatgggcatcgttgcagggtatcccccggacggtgctgctggacaccaagacgggcagcaacctgctgcagtggcccgtggaggaagcggagacgctgcgcaccaactccacggacctcagcggcatcaccatcgactacggctcggcgttcccgctcaacctccggcgcgccacgcagctggacatcgaggcggagttccagctcgaccgccgcgccgtcatgtcgctcaacgaggccgacgtggggtacaactgcagcacgagcggtggcgccgcggcccgcggtgccctcggccccttcggcctgctcgtcctcgccgacaagcacctgcgcgagcagacggccgtctacttctacgtggccaagggcctggacggctccctcaccacgcacttctgccaggacgagtcccggtcctccagcgccaacgacatcgtcaagcgcgtcgtcggcagctccgtccccgtgctggacgacgagaccacgctctcgctccgcgtgctcgtcgaccactccatcgtcgagagcttcgcgcagggcggaaggtcgacggcaacctcgcgcgtctaccccaccgaggccatctacgccaacgccggcgtgttcctcttcaacaacgccaccgccgcgcgcgtcaccgccaagaagctcgtcgtccacgagatggactcatcctacaaccacgactacatggtcacggacatc

SEQ ID NO: 57 - DNA, 519 bp, Sorghum halepense
atggcctcgattctggtgacgacgaccaccgccaccgccatcctcttatgcgtcctcttctgtgccgcggcggctaacaccaccgtcgccaacgaccccaacctccccgactacgtcatccagggccgtgtctactgcgacacctgccgcgccgggttcgtgaccaacgtcaccgagtacatggcgggcgccaaggtgcggctggagtgcaagcacttcggcaccggcgaggtcgagcgcgccatcgacggggtgaccgacgcgaccggcacctacaccatcgagctcaaggacagccacgaggaggacatctgccaggtggtgctggtgcagagcccgcgcaaggactgcgaccagacgcagccgctcagggaccgcgccggcgtcctgctcaccaggaacgtcggcatcgcagacagcctgcgccccgccaacccgctcggctacttcaaggatgtcccgctgcccgtctgcgctgcgctgctcaagcagctggactccgacaatgacgacgatcag

SEQ ID NO: 58 - DNA, 1716 bp, Sorghum halepense
ctgcactgcacgggcacggcgatggtgcgggcgtcgcacactgtgtatccagagctccagtcgttggaggtggagaaggttgatgagatgtcgcgcaccgggtaccacttccagcctccaaagcactggatcaacgatccaaatggaccaatgtactacaaggggctgtaccatctcttctaccagtacaaccccaagggcgcggtgtggggcaacatcgagtgggcgcactcggtgtcaaccgacctgatcgactggacggcactagaccccgggatctacccgtccaagaacttcgacatcaagggctgctggtcaggctccgccaccgtgctccccagcggcatgccgatcgtcatgtacacgggcatcgaccccaacgatcaccaggtgcagaacctggcctaccccaagaacctctccgacccgttcctccgcgagtgggtcaagcccgactacaaccccatcatctcccccgacagcggcatcaacgccagcgccttccgcgacccgaccaccgcctggctcggccccgacaagcactggcgcctgctggtcggcagcagggtcgacgacaagggcctcgccgtgctgtaccggagccgggacttcaagcgctgggtcaaggcgcaccacccgctccactcgggcctcacggggatgtgggagtgcccggacttcttccccgtcgccgtccacggcggcagccgccaccaccgccgcggcgtcgacaccgccgagctgcacgacagggcgctcgccgaggaggtcaagtacgtgctcaaggtcagcctggacatgacgcgctacgagtactacaccgtggggtcctacgaccacgccacggaccgctacacccccgacgccggcttccgcgacaacgactacggcctccgctacgactacggcgacttctacgcgtccaagtcgttctacgatccggccaagcgccgccgcatcctctggggctgggctaacgagtcggacaccgtccccgatgaccgcagaaagggctgggccggcattcaggcgataccgaggaagctgtggctgtcgccgggcgggaagcagctgatccagtggccggtggaggaggtcaaggcgctgcgcgggaagcacgtgaacgtcagtgaccaggtcgtcaagggagggcagtacttcgaggtcgacggcttcaagtccgtgcagtcggacgtggaggtgacgttcgcggtcgacgacctgagcaaggcggagcagttcaacccaaagtggttcactgacccacaaaggctgtgcaagaagcggggcgcccgggagaagggcgaggtgggcccgttcgggctgtgggtgctggccgccggcgacctcacggagaggacggccgtcttcttcagggtgttcaggaccaacaccagcaggctcgtcgtactcatgtgcaacgaccctaccaactccacgttcgaggcgcaggtctaccgccccaccttcgccagcttcgtcaaccacgacatcgccaaaaccaaaaccatcgcactcaggacactgatcgaccactccgtggtggagagcttcggggccggcggaaggacgtgcatcttgtcgagggtgtacccgaagaaggccctgggcgacaacgcgcacctctttgtgttcaaccacggcgaggtggacgtcaaggtcgcgaagctggacgcctgggagatgaggacgccgaagatgaacgcgccggcgcag

SEQ ID NO: 59 - DNA, 408 bp, Sorghum halepense
aaggttatcgatgttgtcgtctccgccgccccaccggacaagcaaaaggaaaccttgcatgccgctcagaagcacctcaaacccctcacttccgctcttgacaaggccaaggagacaggagatgagaaggaaatcgctcgcctcgttctttctgtggagataacccttgccatgaccaagaatgcgccgccagagaagaagctcaagacgatggaggactccatcaactcggtagctgcacctagcccgctagattgccctaccgtcgataaggcttactgtgagatgcacgccaagattcagaaggccgtcaatggattcgccacggctgacctagcaaacaaaatgtcggaggcgcaagctactgtcctcgaggaaacattatacaccgctggctccactatcaac

SEQ ID NO: 60 - DNA, 498 bp, Sorghum halepense
atgacgcagcagtcgatggtggcgctggtcgccgccggcgtcctcctcctcgccggcgtggcgtcggcggagaaggcgggcgggttcgtggtgacgggtcgcgtgtactgcgccccctgccgcgccgggttcgagacgaacgtgtccaagagcgtggcgggcgcgacggtggaggtggtgtgccggcacttcgaggcgagcaaggagacgctcaaggcggaggcgacgacggacgacttcgggtggtacaagctggagatcgaccaggaccaccaggaggagatctgcgaggtggtgctcaagaagagccccgacccggcgtgcgccgagatcgaggagcagcgcgcccgcgcccgcgtcccgctcacctccaacaacggcatcaagcagaagggcacccggtacgccaaccccatcgccttcttccgcaaggacccgctcaaggagtgcggcgcaatcctccagaagtacgacctcaaggacgcatccgacacgcca

SEQ ID NO: 61 - DNA, 1278 bp, Sorghum halepense
agggccccggcaccggcacgcagcctccatccgatccccggcctccagcacgagatacatacaccgatggcgttcatcagcaacgacgtcgtggcgatgaaggcggcggccgtgtccgctctgctggtgttcgcggcggtggcgggaggcgcgccgtcgatgcccgcgggcccgctggacatcgcgcagctgggcgccaagggcgacggcaagtcggacagcaccccgatgatcctcaaggcgtggaagaacgcgtgcgacgcgaccggggtgcagaagatcgtcatcccaccgggcaactacctgacgggcgggctggagctcaagggcccctgcaagtcctccatcatcatccgcctcgacggcaacctgctcggcaccggcgacctcaacgcgtacaagcgcaactggatcgagatcgagaacgtcgacaacctgtccatcaatggccacggcaccatcgacgggcagggctccctggtgtggaacaagaacgactgccagcattcctacaactgcaaggtcctccccaatagcttggtgctggactttgtgacgaacgcccagatcaggggcatcacgctggccaacagcaagttcttccacctcaacatcttcgcgagcaagaacgtgttgatcgacaaggtgacggtgaaggccccggggaacagccccaacacggacggaatccacatgggggactctgagaacgtgaccatcagcggcaccaccatcggcgtcggcgacgactgcatctccatcgggcccggcagcaagacgatccggatcgacggcgtcaagtgcggccccggccacggcatcagcgtcgggagcctggggaggtacaaggacgagaaggacgtggaggacgtgaaggtgaaaggttgcacgctggtgggcaccaccaacggcctgcgcatcaagtcgtacgaggactccaagtcgtcgcccaaggtcaccaagttcgtgtacgaggacgtgaccatggacaacgtctcgtacccgatcatcatcgaccagaagtactgcccgaacaacatctgcgtcaggtccggcgcgtccaaggtggccgtcaccgacgtcgtcttcaagaacatccacggcacctccaacacgcccgaggccatcacgctcaactgcgccgacaacctgccatgccagggcgtgcagctccacaacgtcgacatcaagtacaacaagtccaacaacaagaccatggccgtctgcaagaacgccgtcggcaagtccttcggcttgtcaaaggagctcgcgtgcatc

SEQ ID NO: 62 - DNA, 1338 bp, Sorghum halepense
atggcggtcacgatcacgtgggtgaaggcgaggcagatcttcgacagccgcggcaaccccaccgtcgaggtggacatcggcctcagcgacggcagctacgcgaggggtgccgtgccgagcggcgcatccactggaatatatgaggccttggagttgagggatggaggatctgattatcttggcaagggtgttcttaaggctgtgagcaatgtaaatacaattattggaccagcaatcgttggaaaggaccccactgagcaggttgagattgacaacttcatggtccaacagcttgatggtacctccaacgaatggggctggtgcaaacagaagcttggcgcaaatgccattcttgccgtttcacttgctgtgtgtaaagctggagctatggtgaagaagattcctctttaccagcacatcgcaaatcttgctggaaacaaaactttggtgctccctgtacctgcctttaatgtgattaatggcggatcacatgctgggaacaagcttgccatgcaggagttcatgatcctcccaactggtgcctcctcgttcaaggaggccatgaagatgggagttgaggtgtaccacaacctgaagagcataatcaagaagaagtatggtcaagatgccacaaatgttggggatgaaggtggttttgcacctaacattcaggaaaacaaagaaggccttgaactactgaaggcagctatagaaaaggctggctacactggaaaggtggtcattggaatggatgttgctgcttctgaattctttagtgagaaggacaagacttatgatcttaatttcaaggaggagaacaacgatggttcaaacaaaatttcaggtgacagcctgaaagatctgtacaagtcctttgtgtctgagtaccctattgtgtctatcgaagatccattcgatcaggatgactggaccacttatgccaaactcactgatgagattggacagcaagtgcagattgtaggagatgatcttcttgttactaaccccactagggttgccaaggccatcaatgagaagacctgcaatgctcttctcttgaaggtgaaccaaattggctctgtgaccgagagtatcgaagctgttagaatgtccaagcgtgccggatggggtgtgatggcaagccacaggagtggcgagactgaggacaccttcatagctgacctctcagttggcctggctacgggccaaatcaagacaggagctccctgccggtctgagcgcctggccaaatacaaccagctcctcaggatcgaggaagagctcggtgatgccgcagtctacgccggagcaaagttcagggcaccagtggagccctac

SEQ ID NO: 63 - DNA, 738 bp, Sorghum halepense
atgacctgcaagatgcgacgacgccacgccacgacgacgacgatgatagccgccgtgctctgcctgctcctcttctccggccgcctcgccgcggcggagaagtctttcggtggtggaggctacagcgggttggaggccggtgggcagcagccggagacgggtggagcgagcgaggcggcgcttgccggtgccgctgagacgacgacgacaccggcggcatattcgtcaggcggcgacgctgccgctgcttccggcggcggcggcgggggtggttacggcggcaagctggacccggacggcgacccggaggttggtctgaacgagaaggcgatcaaggagatcgtggacgagcacaacatgttccgcgccaaggagaacgcaggcctgccgccgctggtgtggaacgagacgctggccaagtggtcgcagaagtacgcggagacgctcaagggcaactgccagcagatccactcgacgtcgccgtacggggagaacctcatggagggcacgccggggctcacctggaagatcaccgtcgacggctggagcgaggagaagaagaactaccacttcgactccgacacctgcgacgccggcaagatgtgcgggcactacaaggccgtcgtctggaagaccaccaccagcgtcggctgcggacgcatcaagtgcaacagcggcgacaccatcatcatgtgcagctactggccgccggggaactacgacggcgtcaagccatac

SEQ ID NO: 64 - DNA, 753 bp, Sorghum halepense
atggcgggcaggaagatcctcctcctcctcctactttgtgccatggatagggtagcagtggtagtgctcgccgtcgcagggcagcaaggcgccgatccgcgggcactgccggcggagtgggcgacggcgataaagtacaaggccaccatggacgtgaagacgcgccaggctttcgacggcgtggtggctgccgctccggcggagaagcgttcggaggccgtggaagccgtgctgcagcagcagctcaacatggacgtctccctgggcaaggccacggcgtccggcgacgagaacaacttcgtgagcgtggccggcgcgtacgagaaggcggccgacgccgtcatcgcggcgtcgccggcgaacaagctgggcacgatggcgttcgcgtacaacggcgtggtggcgccggacccgggcaggtgcaccgccgccgccgccgccgacaagcccttctgcgagacgtacgccaagacggagaaggccttcgccggggtcatcgccacgggggactcgccgcggacgaggctggggttcacggacgtggtgctcaagcagaggctcgccaccgacgccgccatcaacaaggcgtacgccgagggggacaaggacaagatcgccaaaatccttgctgcctacgcgcaggccgccgacgcggtcgctgccgccgcgcctcctgaaaagctcagagtcatggagcagaccttctcggcggtggccgccgccgctcatcagcctgcagctgctgcaaaagct

SEQ ID NO: 65 - DNA, 1173 bp, Sorghum halepense
atggaattgcggccgtggctgctcttagtggtcgtcgcgatcgtcgtgtcaatatctgcagacggggggaccgtcataaacgtcaagaactacggggcccatggcaatggcgccaatgacgattccaagccactgatggcggcgtggaaggcagcgtgcggatcagctggcgcggtgacgatggtcgtgacgccggggacgtactacatcggtccggtgcagttccacggcccctgcaaggcctccagcttgaccttccagctgcagggtagtacgctcaaggccgccacggacctgagcaagttcggcaacgactggatcgagttcgggtgggtgaacgggcttaccgtcacaggcggcaccattgacggccagggcgccgcctcgtggcccttcaacaagtgccccgtccgcaaggactgcaaagtcctgcccaccagcgtgctgttcgtgaacaaccagaacacagtggtgcgcgacgtgacgtcagtgaatcccaagttcttccacatggcgctgctgacggtcaagaacatccggatgagcgggctcaagatcagcgcgccgtccaacagtcccaacacggatggcatccacatcgagcgcagcagcggcatacaaatcacggacacgcgcatcagcacgggcgacgactgcatctccatcggtcagggcaacgacaacgtgcaaattgcccgcgtgcagtgcggccccggccacggcatgagtgtcggcagcttgggtcggtatgccagcgagggcgacgtcacacgggtccacgtccgcgacatgaccttcacgggcaccacgaacggcgttcgcatcaagacatgggagaactccccttccaaaagccacgccgcacacatggttttcgagaacatggtcatgaaggacgtccagaaccccattatcatcgaccagaagtactgcccctactacaactgcgagcacaagtacgtgtccggggtgactatccaggacattcagttcaagaacatcaagggcacggcggcgacccaggtggcggtgttactacggtgcggtgtgccgtgccagggtctggtgctgcaggacgtggaccttaggtacaaggggcagggtggtacattggccaagtgcgagaatgccaaggccaagtacgttggcaaccagtttcccaagccgtgcccg

SEQ ID NO: 66 - DNA, 354 bp, Sorghum halepense
gagatcgagggccaccacctcacgtcggcggccatcgccggccacgacggcgccgtctgggctcagagcgccacattccccgagttcaagcccgaggacatgaccaacatcatgaaggatttcgatgagccggggcacctcgcgccgacaggcctgtttcttggagccacgaagtacatggtcatccaaggtgaacccggcgctgtcattcgtggcaagaagggatcaggaggcatcactgtgaagaagacagggcaggcactcatcattggcatctacgacgagccgatgactcccgggcagtgcaacatggtggtggaaaggctgggcgactacctggttgaacagggcatg

SEQ ID NO: 67 - DNA, 243 bp, Sorghum halepense
cgccggcccaagccgtcgtgccacgaggtcatggtcggcgagtaccgccccacggccgccgacgccgcggccaaccggacggcagggttcgggctcgtcaccaacatcgtcaacggcgggctcgagtgcaatcgcaccgacgatgcccgggtcaacaatcggattggcttctaccagaggtactgccagatcttcaacgtcgacgccggcgccaacctcgactgcgctcaccagcagccgtac

SEQ ID NO: 68 - DNA, 1038 bp, Sorghum halepense
ctcttcctcgcgcgcggcgccgtcgtccgcgccacgcaggacacatcaagctggcctctgatcgaaccactgccctcatatgggagaggacgagagctgcccggcggaagatacatcagtttaatccatggcagtgggcttcaggatgttgtcatcacaggtgagaatggaactattgatggtcaaggcaccccatggtgggatatgtggaagaagggcacactgctctacacaaggcctcaccttcttgagctgatgagctcttctcatatcatcgtctccaatgttgtctttcaggattcaccattctggaacattcatcctgtttattgcagcaatgttgtgatcagaaatgtgactatcctagctccacatgactcccccaacacggatggaattgatccagattccagcagcaacatctgcattgaggattgctacatttcgacgggtgacgatgccattgccatcaagagtggctgggatgagtatggaatcgcttatggccgccccagctccgacatcaccgtccgccggatcacaggctcctccccttttgccggcttcgctgttggaagcgaaacatcaggtggcgtggagaacgtccttgcagagcacctgaacttcttcagctcagggttcgggatccacatcaagaccaacaccggcaggggaggcttcatccggaacgtcactgtctccgacgtgaccctggataacgtccgctacggcctgcggatcgtcggcgatgttggcaaccaccccgacgagcgctacaaccggagcgcgctcccaatcgttgacgccctgacgataaagaatgtccagggccagaacatcagggaggctgggctgatcaagggcattgccaactcggccttctcccggatctgcctgtcgaacgtcaagctcactggaggtgcgcctgtccagccgtggaagtgcgaggctgtcagcggtggcgctctcgacgtgcagccgtcgccgtgcacagaactgacttccacgtccgggacgagcttctgtacaaattcactc

SEQ ID NO: 69 - DNA, 381 bp, Sorghum halepense
atggcaatggccaagatcctcctgctcatcctcctcgtcgtcgtcactgcggtcgtagaggccgccgatccaccggcgaaatggaaggccgccctgacggccttagacgccatggacgcgaagatgcggcaagctgttgatggcgtcgccgccgccgctccagccgagaagcagtccgaggtccaagaggccgccatggcggagaggctggatgtctcactcgccctcgcccgggtcgaagaaaccgggaacgagaagaaggtcgagagcatggcggcttcctacgagaaagccgccgacctggttgtcgccgcgccaccgcctgacaagctcaaagtcatgaaggaggccttccgcgcggtaacaaaagcagcagcgtta

SEQ ID NO: 70 - DNA, 378 bp, Sorghum halepense
atggcgactacggaggcggcggcggcgacaccggtggcgccggcagaggggtcggtgatcgcgatccacagcctcgacgagtggagcatccagatcgaggaggccaacagcgccaagaagctggtggtgattgacttcactgctacttggtgtccaccatgccgcatgatagctccagtttttgctgaactggccaagaagcacccaaatgttgttttcctgaaagttgatgttgatgaaatgaagaccattgctgagcaattcagtgttgaggccatgccaacattcctgttcatgagggagggcgacgtcaaggacagggttgtcggcgcagcaaaggaagagctagcaaataagcttcaactgcagatggcccag

SEQ ID NO: 71 - DNA, 393 bp, Sorghum halepense
atgtcgtggcagacgtacgtcgacgagcacctcatgtgcgagatcgagggccaccacctcacctccgccgccatcatcggccacgacggcaccgtctgggcccagagcaccgcgttcccgcagttcaagcctgaggagatgaccaacatcatgaaggacttcgacgagcccgggttcctggccccgaccggcctcttcctcggccccaccaagtacatggtcatccaaggcgagcccggcgccgtcatccgcgggaagaagggatctggaggcataaccgtgaagaagaccgggcaggcgctggtgatcggcatctacgacgagcccatgacccccgggcagtgcaacatggtggttgagaggctcggcgactacctcgtagagcaaggcctg

SEQ ID NO: 72 - DNA, 1239 bp, Sorghum halepense
atggcttccgcaagcaatgctctcagggtgttcttcatcctagcgatcctatgcgcggtatgcacagcgaaaaggaccggagcaaagacgggagactcggcagcagactctgctgcttctggagccagcgggacgttcgacatctccaagctcggcgcgacgggcgacggcaagacggactccacaaaggcggttcaagacgcgtggacgtcagcgtgcagagcgaccggaagcgccacggtgctcatccccaagggcgactatctggtcggccctctcaacttcgttggcccgtgcaagggcgccatcaccatccagctcgatggcaacctgctgggatccaacgacctggccaagtacaaggcgagctggatcgagttgtcgcacgtcgacaacatcgtcatgactggctcgggcacgctcgacggccagggcaccgccgtttataaaaaggccaaaaccggcactgtgaaggcgatgcccaacacattggtgctgttctacgtgaccaacggcactgtctctggaatcaaactactcaactccaagttcttccacatcaatatcgacgcttcaaagaacatcacggtgaaggacgtgaacatcaccgcgcctggggacgttgagaacacggacggcgtccatgttggaatgtccaccaaggtgagcatcaccaactcaaccatcggcactggcgacgactgcatctctgttggccccgggagcgacggcgtcatggtgaacaacatcatctgcggccccgggcagggcatcagcatcggctgcctaggccgctacaaggatgagaaggacgtgaccgacgtgacggtgcgggactgcgtgctcaagaagaccactaacggcgtgcgcatcaagtcgtacgaggacgccgagtctgtgctgacggcgtcgcatctgaccttcgagaacatcaggatggaggaggtggcgaaccccatcatcatcgaccagtacttctgccccgagaaggtgtgccccggcaagaagagcgactcctctcatgtcatcgtcaaggacgtcacgttccgcaacatcacgggcacgtcgtccacgccacaggccatcagcctgctctgctcgcagtcacagccatgcagcggcgtgtcgctcatcgatgtaaacgtggagtacgccggcaagaacaacaagaccatggccgtgtgcagcaacgccaagggcaccgccaagggcagcgtcgaggccctggcatgcctggcc

SEQ ID NO: 73 - DNA, 819 bp, Sorghum halepense
atgtctggtcgtcgtcgtcgttcgtggacaatatgggcgcctctcctggcgtcgctgctgctcgccgggctggcgctgtcggccaaggtggtggacgaggaggaggcggaggcggacggcgacgacggtggtggcgccagcaagaagaagaagccccacgttaaccacggcaagttcaaggcggacccgtggacggacgggcacgcgacgttctacggcggccgcgacgggtccggcaccacggacggcggcgcgtgcggctacaagggcgagctgggaaaggactacggcgcgctcacggcggccgtgggcccgtcgctctacaccaacggcgccgggtgcggcgcgtgctacgagctcaagggctccaagggcaccgtggtcgtgacggccaccaaccaggccccgccgccggtcagcgggcagaagggcgagcacttcgacctcaccatgccggcgttcctcaagatcgacgaggagaaggccggcatcgtgcccatcacctatcgcaaggtggcgtgcgcgaggcaaggcggcatccggtacaccatcacggggaacccgaactacaacatggtgatggtgaccaacgtgggcggcgccggggacgtggtggcgctgtcggtgaagggcaacaagcgcgtcaagtggacgccgatgaagcgcagctggggacagctctggatcacggaggtcaacctcaccggcgagtcgctgacgttccgcgtcatgaccggcgaccaccgcaaggctacctcctggcacgtcgcgccccgcgactggacgtacgacaaaacataccaggccaccaagaacttc

SEQ ID NO: 74 - DNA, 1326 bp, Sorghum halepense
atgatgatggacgcccgcctccgccgcctcgtcttcctcctcctcctcgcggccgcggcgccgctggccaccgcccagctctcacaagatttctacaagacgtcgtgccccgacgccgagaagatcatcttcggcgtcgtcgagaagcggttcaaggcggaccccggcaccgccgccggcctcctccgcctcgtcttccacgactgcttcgcaaacggctgcgacgcgtccatcttgatcgacccgatgtcgaaccaagcctccgagaaggaggccggtcccaacatctccgtcaagggctacgacgtgatcgaggagatcaagacggagctggagaagaagtgccccggcgtggtgtcgtgcgcggacatcgtgtcggtgagcgcccgcgactcggtgaagctgacgggagggcccgagtactcggtgcccctggggcggcgcgactcgctcgtgtccaaccgcgaggacgccgacaacctgcctggcccggacatcgcggtgcccaagctcatcgacgagttctccaagcagggcttcaacctcgaggagatggtcgccatgctgggcggcggccacagcatcggcatctgcaggtgcttcttcatcgagaccgacgcggcgcccatcgaccccgggtacaagaagaagatcagcgacgcctgcgacggcaaggactcgggctccgtcgacatggactccacctcgcccaacaccttcgacggcagctacttcggcctcgtcctggagaagaagatgccgctcaccatcgaccgcctcatggggatggactccaagacggagcccgtggtgcaggccatggccgacaagaagaccgacttcgtccccatcttcgccaaggccatggagaagctcagcaacttgaaggttatcacggggaaggacggcgagatcaggaaggtgtgctccgagttcaacaacccgcagaacagcagcagcagctcgtcggtgatacggaccagctccgtcaacgccgacgaggtggccggcctgtcgtcctcgtccagcaggaaggtgggccctcccgacgcggtggaaacacccgccatggtggcggatgaggcggccgccaaggttcccggcggtgtcgtcgtcagcgtcgggggagaccagcagcagccgccaaacccggaagccgataggcccggcctgaagctccgcggcagccgcgaacctgtgaacccggcggccccggtggagcccggccccggcggcgaggatgctgccaagcagcaggcggtggcggccctggaggagaagaagaagaggaacatggccaaactccgggccgcccaggccaagatg

SEQ ID NO: 75 - DNA, 729 bp, Sorghum halepense
atggccaagatgatcctcctactaatcatccttgttgctaccatcacagccgccgtggaagccgctccttcgccagcagtgcctgtgcctcagcaatcctccgccgaagctgacaagaagatcaacgaggtgaatctgacactgaagaaggtcttcgacgatgtcatcgccaccgccccgccggccaagaagcaggaagccatagacgcgaccaccaagcagctccaagtcgccgagcgcgccctcgcgaaggccaaggcaggaggcgacgagaaggtcgcgaagctcgccatgtcctacgagctgagcgccaggatcgtcacggagacaccgccggcgatgaagctggagcggatggaggagctgttcaacgccatggctgcaccgaaccacaagacagaatgccacccgaacgccgaggccgacaagcccttctgcgagaccgtctccaagctgcagaaggccttcaaggaggtgcgctccgccgtcgcgcagggcaagaaggaggagaccatcgacgatgtgttcctcgtcaaccaagagttcgcgcccacgatcagggctatcaacaaggcgtacgcggacggagacgagaaggagatcgcggcggttctggcgacctacgacaagtgtgccgatgcgatccttgcagctccgctcgctgaaaagttcaaggtgatgaaagagagcatcgcggccgcttcccgtgcacccggaaagcaggcgcca

SEQ ID NO: 76 - DNA, 240 bp, Sorghum halepense
atggcggagacggcggacatggagcggatcttcaagcggttcgacaccaatggcgacggcaagatctcgctgtcggagctgacggacgcgctgcggcagctggggtccacctccgccgacgaggtgcagcgcatgatggccgagatcgacaccgacggcgacggctgcatcgacttcaacgagttcatcaccttctgcaacgccaacccggggctcatgaaggacgtcgccaaggtcttc

SEQ ID NO: 77 - DNA, 432 bp, Sorghum halepense
atgatgccgcagctgcgtagcctcgtcgcgctgctcctagtggccacggcagtcgccgccgtcgtggccgtcgccgcagccggtggtgggtttgtcgtcaccggccgcatctactgcgacaactgccgcgccgggttcgagaccaacatctcccacgccatccaaggcgcgacggtggagatggagtgccgtcacttcgagtcgcagcagattcacgacaaggcacaggcgacgacggacgccggcgggtggtacaagatggagatcgccggcgaccaccaggacgagatctgcgacgtgaggctgctcaagagccccgaggcggactgcgccgagatcgaggtcaaccgcgacaggtgccgcgtcccactcaccggcaacgacggcatcaagcagagcggcgtccgatacgccaaccccatcgccttcttc

SEQ ID NO: 78 - DNA, 660 bp, Sorghum halepense
ccttcaagcgtcgtggttctgactccagagacttttgactctattgtccttgatgaaaccaaagatgtccttgttgagttctatgctccatggtgtggtcactgcaagagtcttgcaccgacatatgagaaggtggcttctgttttcaagttagatgaaggagttgttattgctaaccttgatgctgacaaatacagggatttggctgagaagtatggagttactggatttcctacattgaagtttttcccaaagggaaacaaagctggtgaagattatgatggcggtagggacttgggtgactttgtcaagttcattaatgagaagagtggtaccagccgtgacacaaagggtcaactaacctcagaggctggccgcatagcaagtctggatgtcctggctaaggagttccttggtgcttccagtgacaagcgaaaggaagtcctttccagtatggaggaggaggcagctaagctcagtggtccttctgcgaggcatgggaaggtctatgtaaacatcgcaaagaagattctagagaaaggcaatgagtatactaagaaggaaaccgagaggcttgatcgcatgttggagaagtccatcaacccctcgaaggccgatgagtttatcatcaagaagaatgttctctcgactttctcatcc

SEQ ID NO: 79 - DNA, 363 bp, Sorghum halepense
atgacctcgtcatcctccttcctgctcgtggtggtggtgttggcagcactgtttgccgtcagctcgtgtgacaaccccccggccatcaccttcacgatcggtaaggactccagctccaccaaactatcctttgccaccgacgtcgccatctccgaggtggcggtcaagcagaatggcgccgagaattggtctgacaacctcaaggagtcgcccgtcaagacctttaccctcgacagcaaggatccgatcaagggacctatcaccatccgcttcgctgataaggatggtggctaccatgttcttgttgatatcatccctgccgactttaaggctggctcagtttataaggctttgtcctacgtc

SEQ ID NO: 80 - DNA, 1389 bp, Sorghum halepense
atggcaccgggaagatctcttcgccctgcggtggcggtggttctgtgggcggcgtcgctggtgctgctgctggcggcggcgtgcgcgggcgccggcggcgcgtccggggggccagggtgccggaagcacgtggcgaaggtcacggagtacggcgcggtgggggacgggaggacgctcaacacggaggcgttcgccaaggcggtggcggacctgtcgcggcgcgcccgcgatggcggcgcggcgctggtggtgccgccggggaagtggctcacggggcccttcaatctcaccagctgcttcacgctctacctcgacgagggcgccgagatcctagcgtcccaggacatgaaccattggcccctcatagctcccttgccgtcttacgggagaggaagggacgagcctggcccaaggtacatcaatttcattggaggatccaatctcactgacgtcatcatcacaggtaaaaatggaacaatcaacgggcaggggcaagtctggtgggacaagttccatgccaaggagctcaagtccacccgtggccacctcctagagctcctccactctgataacatcatcatctccaatgtcaccttcgtcgatgcgccatactggaacctccaccctacctattgcaccaatgtgaccatcagtggcgtcaccattctcgcgccattgaattcgcctaacaccgatggaattgacccagactcttccacgcatgtgaagatcgaggactgctacatcgtctccggcgacgactgcgtcgccgtgaagagcgggtgggacgaatatggcatcaagttcaacatgccgagccagcacatcgtcatcaggaggctgacctgcatctctcccacgagcgccatgatcgcgctgggcagcgagatgtctggcggcatccgcgacgtgcgcgccgaggacaacatcgccatcaacacggagtcggccgtcaggatcaagtccggcgcggggaggggcggcttcgtcagggacatcttcgtgcgccgcctcagcctccacaccatgaagtgggtgttctggatgaccggcaactacgggcagcaccccgacaacacgtccaaccccaacgccatgcccgaggtcaccggcatcaactacagcgatgtgttcgccgagaacgtgaccacggccggcaggatggagggcatccccaacgacccatacaccggcatctgcatatccaacgtgaccgccagcctcgcgccgaacgccacggagctgcagtggaactgtaccaacgtcaagggggtcacctccaacgtctcgcccaagccatgcccggagctcggcgcggagggcaagccgtgcgccttcccagtggaagagctcgtcatcggcccaccggcgctgccaaagtgtagctac

SEQ ID NO: 81 - DNA, 528 bp, Sorghum halepense
atgatgggacgacggtgcataacagatcaggtgctgctcgccgcggtggtggtggtggcggcggccagcggcggcctgctggtggccgcggtgaaggacgacgacttcttcgtggacggctcggtgtactgcgacacgtgccgggcgggtttcgagacgaacgcgacgacgccgatcgcgggcgccaaggtgcgtctggagtgccggcactacatgagcgcgagcggcgcggtggagcggtcggcggagggcgccacggacgcggcggggcggtaccgcatcgagctggtggacaaccgcggcgccgaggaggtgtgctcggtggtgctgctcagcagccccgtgcccgggtgcgccgagaaggaggtgggccgcgaccgcgcgcaggtggagctggtcacggacgccggcgccgggctcgccaccaccgtgcgccgcgccaacccgctgggcttcctcaagagtcagccgctccccaactgcggcgagatactcaagagctacgggcttggctccgggcctggctac

SEQ ID NO: 82 - DNA, 549 bp, Sorghum halepense
cgagtcctgacgaaatctgaccaccccggttcacactgcgctgctccgcccgccatggcgatccgctcctccaaggcctgctggatctcgctgctgctcgcgctcgcgctctccgccgtcgcgcgggcggaggagcccgcggcggagggtgcggccgaggccgtgctcacactcgacgtcgacagcttcgacgaggccgtcgccaagcacccgttcatggtcgtcgagttctacgccccctggtgtggacactgcaagaagcttgctccagagtatgagactgcggccaaggaacttagcaagcacgacccaccgattgttctcgctaaggttgacgctaatgaggagaagaacaggccgcttgctaccaagtacgagatccaagggttcccaaccctcaagatcttcaggaaccaggggaagaacattcaggaatacaagggccccagggaggctgatggcattgtcgattacttgaagaagcaggttggccctgcgtccaaggagctcaagtcacaggaagatgttgcgacccattatgatgacaag

SEQ ID NO: 83 - DNA, 444 bp, Sorghum halepense
atgggcggcaaggacctgacggaggaccagatcgcctcgatgcgggaggcgttctcgctgttcgacacggacggggacggcaagatcgcgccgtcggagctgggcgtcctgatgcgctccctgggcgggaaccccacgcaggcgcagctccgtgacatcgcggcgcaggagaagctcacggcgcccttcgacttcccgcgcttcctcgagctcatgcgcgcccacctcaagcccgagcccttcgaccgcccgctccgcgacgccttccgcgtcctcgacaaggacggctccggcaccgtctcagtcgccgacctccgccacgtcctcacctccatcggcgagaagctcgaggcgcacgagttcgacgagtggatccgcgaggtcgacgtcgcccccgacggcaccatccgctacgacgacttcatccgccgcatcgtcgccaaa

SEQ ID NO: 84 - DNA, 312 bp, Sorghum halepense
gaaaggctgggtgcaagttttaagaaagctaaatctgtcttgattgctaagattgattgtgatgagcacaagagtttgtgcagtaagtatggagtttccgggtatccaacaatccaatggttcccaaaaggatccttggagcccaaaaagtatgaaggacaacgcactgcagaagcccttgctgaatttgttaatactgaaggaggcacaaatgtaaagctggcaaccattccttcaagtgttgtggttctgaccccggagacctttgactcaattgtccttgatgaagccaaagatgtccttgttgagttc

SEQ ID NO: 85 - DNA, 324 bp, Sorghum halepense
tggtgtggacactgcaagaagctggcgcccatcttggaagaggcagccaccactctccagagtgatgaggaagttgtgattgctaagatggacgcgactgccaatgatgtgcccagtgagttcgaggtccaaggctacccaaccatgtacttcgtgacccccagcgggaaggtcaccgcctacgacagcggcaggacagcagacgacatcgtcgacttcatcaagaagagcaaggagaccgccggcgccacccaggcgacgacgacgacatctgagaaggcagctgacgcagccgagaaagctgagcccgtcaaggacgagctg

SEQ ID NO: 86 - DNA, 1578 bp, Sorghum halepense
atggcgacatcaaggtcccttgccttggcgctcctcttgtgcgccttgtcatcctcctgccacgccgccatttcctacccaccgtcggccatgcccgccgccgcgcccgccaaaggtgacttcctcgcgtgcctcaccaagagcatccccccgcggctcctctacgccaggagctcgcctgcgtacggctccatctgggcgtccaccgtccggaacctcaagttcgactcggacaagacggcgaagccactgtacatcatcaccccgacggagcccgcccacatccaggccaccgtggcgtgcggcaggaagcacggcatgcgggtccgcgtgcggagcggcgggcacgactacgagggcctgtcgtaccgttccaccaagccggagacgttcgccgtggtggacatgtccttgctacggaaggtgtcgctggacggcaaggcggccacggcgtgggtcgactccggcgcgcagctcggcgacatctactacgcgctggggaagtgggcacccaagctcgggttcccggcgggcgtgtgcgccaccatcggcgtcggggggcacttcagcggtggcggcttcggcatgatgctgcgcaagcacggcctcgccgtggacaacgtcgtcgacgccaaggtggtggacgccaacggcaacctgctggacaggaagaccatgggcgaggactacttctgggcgatcaggggcggcggcggcgagagcttcggcatcgtggtgtcgtggcagctgaagctcgtgcccgtcccgcccaaggtgacggtgttgcagatgcccaggagtgtcaaggacggcgccatcgacctcatcgtcaaatggcagcaggtggcgccgtcgctccccgaggacctgatgatccggatcttggccatgggaggcaccgccatattcgagggcctgttcctcggcacgtgcaaggacctcctcccgctgatggccagccggttcccggagctgggcgtgaagcaaggggactgcaaggagatgtcgtgggtgcagtcggtggcgttcatccccatgggcgacaaggccaccatgaaggacctcctgaaccggacgtccaacatcaggtcgttcggcaagtacaagtcggactacgtcaaggaccccatcgcgaagccggtgtgggagaagatctacgcgtggctggccaagcccggtgccgggatcatgatcatggacccctacggcgccaagatcagcgccatcccggacagggcgacgccgttcccgcaccggcaggggatgctgttcaacatccagtacgtcacttactggtccggcgaggccgccggggcggcgccgacgcagtggagcagggacatgtacgcgttcatggagccgtacgtgaccaagaacccgaggcaggcgtacgtcaactacagggacctcgacctcggcgtcaaccaggtggtcaacgacatctccacctacgagagcggcaaggtctggggagagaagtacttcagcttcaacttcgagaggctcgccaggatcaaggccaaggtggaccccaccgactacttcagaaatgagcagacaatcccaccattgttcaag

SEQ ID NO: 87 - DNA, 597 bp, Sorghum halepense
atggactcgaaccagggcgtggtggcggtggtgaagccgacgctggccaaggggacgccgtcggcgtcgttccggctccgcaacgggagcctgaacgcggtgcgcctccgccgcgtgttcgacctgttcgaccgcaacggggacggcgagatcacggtggacgagctggcgcaggcgctggacgcgctgggcctggacgccgaccgcgccggcctggccgccaccgtgggggcctacgtgcccgacggcgccgcgggcctccgcttcgaggacttcgacaagctccaccgcgcgctcggggacgccttcttcggcgcgctcgcggaccaccaggacgacgccacggacgccggcggcaagaagggggaggaggacgagcaggagatgcgggaggcgttcaaggtcttcgacgtcgacggcgacggcttcatctccgccgccgagctgcagacggtgctcaagaagctgggactccccgaggccagcagcatggccaacgtccgggagatgatcaccaacgtcgaccgcgacagcgacggccgcgtcgacttcagcgagttcaaatgcatgatgaaggggatcaccgtctggggcgcc

SEQ ID NO: 88 - DNA, 450 bp, Sorghum halepense
gagaccactggcccaaacatggttgttgatatgtgtaagggagtgcagtatctcaatgaaatcaaggattctgtcgtggctggtttccagtgggcatcaaaggagggtgcactggctgaggagaacatgcgcggaatttgctttgaggtctgtgatgtcgttcttcatgctgatgctatccacaggggtggtggccaggtcatcccaactgccaggagggtcatctatgcttctcagctcacggccaagccaaggctgctggagccagtgtacctggtggagattcaggccccagaaaatgcacttggtggtatctacggtgttctgaaccagaagagagggcatgtgtttgaggagatgcagaggccgggtaccccgctctacaacatcaaggcttacctccctgtcatcgagtcgtttgggttctccagccaactgagggctgcaacc

SEQ ID NO: 89 - DNA, 618 bp, Sorghum halepense
cacggcgccttgtggatcgccgtggtcgctttccttgttgcttccggcagcgtcgtcgtcatccgagtagcggaggcgaggtacggccctggccactggaaccctgccgcccctgcccctgtggcgaccctcgtcagcgagcagctgtacaactccctgttcctgcacaaggacgacgccgcctgccccgccaagggcttctacacctacgccgccttcatccaggccgccaggacgttccccaagttcgccgccacgggcgacctgagcacccgcaagcgcgaggtcgcggccttcttcgcgcaaatctctcacgagaccacaggcggctgggcgacggcgccggacgggcagtacgcgtggggcctgtgctacaaggaggagatcagcccggcgagcagctactgcgacgcgacggacaagcagtggccgtgctacccgggcaagtcctaccacggccggggccccatccagctgtcgtggaacttcaactacgggccggcggggcaggcgctgggcttcgacggcctgcgcaacccggaggtggtggccaactgttccgagaccgcgttccggacggcgctgtggttctggatgacgccgcgccggcccaagccg

SEQ ID NO: 90 - DNA, 357 bp, Sorghum halepense
atggcctcctcgtcctccttcctgctcgccatggcggcgctagcggcattgttggctgttgggtcgtgcagcaccctaatgacctggacgatcggcaaggactccacctccacccgcctcgtcctcgtcgccagcgccgacgtctctgaggtggccgtcaaagacaagggcgccacggatttctcagacgacctcaaggagtcaccagccaagacatttacatacgagagcaaggaaccgatcaagggccccctctccgtccgctttgctgtcaagggtcaaggctaccgcaccaccgacgatgtcatccctgccgacttcaagcctggctcagtttacaagactaaggaacaggtt

SEQ ID NO: 91 - DNA, 363 bp, Sorghum halepense
atggcctcctcatcgtccttctgcttcctactcgcggtggtggcattggcggcattgtttgccatcggctcgtgcggcactacgctcaccatcgaggtcggtaaggactccacctccaccaaattatcccttatcaccaacgtcgccatctccgaggtgtcggtcaagcccaagggagccacggatttcacggacgacctcaaggagtcagaacccaagacttttacgctcgacagcaaggagccgatcgagggacctatcgccttccgcttccttgcgaagggtggtggctaccgtgttgtcgataatgcgatccctgccgacttcaaggccggctcagtttacaagaccaccgaacaagtc

SEQ ID NO: 92 - DNA, 363 bp, Sorghum halepense
atggcatcctcgtcctccttcctgctggccatggcagcgctggcagcattgtttgctgttggattgtgcggcaacagtgtgaccttgacggtcggcaagggcactacccccacgtacacccacctagtccttgtcgccaacctccccatccatgagctggccatcagagaaaagggcgccgcggaatttttggatgacatgaaggagtcaccagccaagacctttacacaagacagcaaggcaccactcaagggacccctctccgtccgctttgctgtgaagggtggtggctaccgcaacagggatgagattttccctgttggattgaagcctggcgcagttatcaacactaacatcccgtat

SEQ ID NO: 93 - DNA, 684 bp, Sorghum halepense
atggcgaagactacgatcctcctcctccttagtagtatccttgttgtcgcggatcaagctgctacaacggcaacacaattgtctcctgccgctgaaaagtccattggcgacctcaatttagaggttgagaaggttatcgatgttgtcgtctctgctgccccacctgcgaagcagatggaaacaatgcatgccgctttgaagcatctccaacccatcaaatctgctctcgccaaggccaaggagtcaggagatgagaagaaaatcgccaaactcgtctttagggtggagatagccgctgccatcatcaatgctgcgccggcagacaagaagctaatgatgatggaggactccttcaactcggtagctgcacccagcccgctagattgccccaccgtcgacaaggcctactgcgagacggactccaagattcagaaggcctttgatggagtcgtcgcggctgccccagtagagaaaaggttggaggtcagagctaccatcctcaagaaaacaatgtacaccgctggctccactatcaacaaggcgtatgcggatggagatgagaaaaagatcgctcaagtccttgctgcctacagtaaggcagctgatgaggtcattgcagccgctcctgctgacaagctcactatcatggagaagacgttcattgctgccgccgccactgggaac

SEQ ID NO: 94 - DNA, 621 bp, Sorghum halepense
atggcttcttcttcctcctccaatcatagcttcctgctgctgcttttattccctctcctgctggtgctgctcctctccacaacggtcgtcgccgccgattctacagctacagcagaaaagtaccagaagtggtgccaagtagcatcagattctccgtcgtgcgtcaaggtgattgagtccatcccaggaatccaggaggtcgattacaataacgccggcaaggtcgccgacttgtgcttgcattttgctgccaataagacaaaggaggccaagggagctgctgacaccttgcttgccgcagaaaaaggcaagcctgcttccggttgcctcaaggcctgcgccaccaacatcaaatcaatggccgaggtattggtcaatcttcctgctgggcaggacgacatgaacgcctatcaaacctataaggaggtcagagccaagttcaaaaacgagaaaccaccagcctgcgagaaggactgctggaacaagacgtcatcgtcgtcatcttcagccgccgacatcgtggacaagttccatgatatctggaatgtggccaaagttgggaatatgcagattaattatatctttccttggccagatagcgacgacgacgataacatcctt

(19) United States
(12) Patent Application Publication
(10) Pub. No.: US 2016/0144020 A1
(43) Pub. Date: May 26, 2016

(54) JOHNSON GRASS ALLERGENIC POLLEN PROTEINS, ENCODING NUCLEIC ACIDS AND METHODS OF USE

(71) Applicant: QUEENSLAND UNIVERSITY OF TECHNOLOGY, Brisbane, Queensland (AU)

(72) Inventors: Janet Davies, Kenmore (AU); Bradley Campbell, St Lucia (AU)

(73) Assignee: Queensland University of Technology, Brisbane, QL (AU)

(21) Appl. No.: 14/899,538

(22) PCT Filed: Jun. 18, 2014
(86) PCT No.: PCT/AU2014/000630

§ 371 Date: Dec. 17, 2015

(30) Foreign Application Priority Data
Jun. 18, 2013 (AU) 2013902260

(51) Int. Cl. A61K 39/36; G01N 33/68

(52) U.S. Cl. CPC A61K 39/36; G01N 33/6893; G01N 2800/24; G01N 2333/415

(57) ABSTRACT

Allergenic Johnson Grass proteins, antibodies thereto and encoding nucleic acids are provided, which may be used for the diagnosis and/or therapy of sensitivity to these allergenic proteins or to immunologically cross-reactive allergenic proteins. In particular, the allergenic Johnson grass proteins and nucleic acids may be used for environmental testing for airborne allergens and/or for batch standardization of diagnostic and therapeutic compositions.

### TECHNICAL FIELD

THIS INVENTION relates to grass pollen allergens. More particularly, this invention relates to isolated allergenic proteins and nucleic acids from the pollen of Johnson grass (Sorghum halepense) that may be useful in diagnosing, preventing and/or treating allergic rhinitis and environmental allergen detection.

### BACKGROUND

Allergic Rhinitis (AR) has increased globally over several decades in both developed and developing nations placing a substantial economic burden on healthcare budgets (World Allergy Organization, White Book on Allergy, www.worldallergy.org). AR causes a negative effect on quality of life, work productivity, depression and anxiety levels of 500 million sufferers worldwide (Brozek et al., J Allergy Clin Immunol 2010; Bousquet et al., Int Arch Allergy Immunol, 2009; Katelaris et al., Clin Exp Allergy, 2012). In Australia, a nation of 23 million people, the direct and indirect cost of allergic disease was a staggering $7.8 billion in 2007 (Cook et al., Australia: Report by Access Economics, 2007). Likewise, in the United States, the direct costs of AR exceed $11 billion per annum (Meltzer and Bukstein, Ann Allergy Asthma Immunol, 2011). Airborne grass pollen levels also affect hospital admissions for asthma (Bauchau and Durham, Eur Respir J, 2004; Erbas et al., Clin Exp Allergy, 2007; Linneberg et al., Clin Exp Allergy, 2007).

The sources of grass pollen allergens vary according to climatic region and it is clear that subtropical grass pollens are clinically important in the subtropics (Phillips et al., Ann Allergy, 1989; White and Bernstein, Ann Allergy Asthma Immunol, 2003; Davies et al., Clin Transl Allergy, 2012). Until this time, most research has concentrated on the temperate grass species of Timothy grass and Ryegrass. Nonetheless, the contribution of subtropical grasses to allergic respiratory diseases of AR and asthma is predicted to increase with a rise in global temperatures due to anthropogenic climate change that may potentially augment the growth range for subtropical grass species (Morgan et al., Nature, 2011; Beggs and Bennett, Asia Pac J Public Health, 2011; Ziska and Caulfield, Aust J Plant Physiol, 2000). Current demographic data indicates that the population of subtropical climates is increasing in size (Gupta, Geology, 2002) and the tropical zones are widening polewards (Seidel et al., Nat Gosci, 2008). For instance, a conservative estimate of the population in subtropical states within the USA stands at ˜52.3 million, having increased by ˜18.3% since 2000 (US Census Bureau for FL, LA, MS, and TX). Changes in the distribution of the human population are concomitant with the exposure of the population to environmental factors restricted to such regions.

Tablets for sublingual immunotherapy (SLIT) for grass pollen allergy are derived from whole pollen extract exclusively from temperate grass species (Pooideae subfamily) (Bufe et al., J Allergy Clin Immunol, 2009; Didier et al., J Allergy Clin Immunol, 2007). Debate persists as to whether single or multiple allergenic extracts of temperate grass pollens endemic to regions of the northern hemisphere are sufficient to effectively tolerize allergic responses to all grass pollen allergens. Furthermore, emerging evidence indicates that sub-tropical pollen allergens show distinct immunological reactivity from temperate grass pollens (Weber, Ann Allergy Asthma Immunol, 2007; Weber, Curr Opin Allergy Clin Immunol, 2005). Allergenic molecules derived from subtropical grass species differ significantly in primary amino acid sequence and immunological reactivity (Davies et al., Allergy, 2005; Davies et al., Mol Immunol, 2008; Davies et al., Mol Immunol, 2011). The immunological relationship between temperate grass pollen allergens and subtropical grass pollens have been explored for Cynodon dactylon (Bermuda grass; Chloridoideae) (Weber, Ann Allergy Asthma Immunol, 2007; Weber, Curr Opin Allergy Clin Immunol, 2005) and Paspalum notatum (Bahia grass; Panicoideae) (Davies et al., Mol Immunol, 2011).

Johnson grass (Sorghum halepense) is a perennial weed distributed throughout the subtropics and tropics, in particular parts of Australia, Africa, Asia and the Americas (Davies et al., Clin Transl Allergy, 2012; Holm et al., The World's Worst Weeds, 1977; McWhorter, Rev Weed Science, 1989). We have previously shown that 77% of patients with allergic rhinitis from a subtropical region of Queensland demonstrate a positive skin prick test (SPT) response to JGP (Davies et al., Clin Exp Allergy, 2011). Thus far, the sequence of the group 1 allergen of JGP, Sor h 1 has been described and shown to react with group 1-specific monoclonal antibodies (Avjioglu et al., Molecular Biology and Immunology of Allergens, 1993).

The allergenic proteins and their encoding nucleic acid from the pollen of Johnson grass (Sorghum halepense), a wind pollinated perennial grass found worldwide and considered a major weed and significant source of allergenicity in the subtropics including parts of Australia, Africa, Asia and the Americas, remain largely undefined.

### SUMMARY

The invention is broadly directed to allergenic proteins and encoding isolated nucleic acids from the pollen of Sorghum halepense (Johnson grass) and/or their use in diagnosing, preventing and/or treating allergic rhinitis.

In a first aspect, the invention provides a method for determining or monitoring sensitivity to a Johnson grass (Sorghum halepense) pollen allergen, or an allergen immunologically cross-reactive with a Johnson grass pollen allergen, in a subject, including the step of determining a presence or absence of an allergen-specific immune response in said subject, wherein the presence of said immune response indicates sensitivity to the Johnson grass pollen allergen or the allergen which is immunologically cross-reactive to the Johnson grass pollen antigen.

Suitably, sensitivity to the Johnson grass pollen allergen and/or the immunologically cross-reactive antigen is associated with an allergic condition.

Preferably, the allergic condition is allergic rhinitis, allergic dermatitis or allergic asthma.

In one embodiment, the subject is a human.

In a second aspect, the invention provides a method for measuring the level of, or detecting or monitoring the presence of a Johnson grass pollen allergen, or an allergen immunologically cross-reactive with a Johnson grass pollen allergen, in a sample, including the step of contacting the sample with one or more reagents for a time and under conditions sufficient to detect said Johnson grass allergen or said immunologically cross-reactive antigen.

In particular embodiments, the one or more reagents comprise an antibody or fragment thereof.

In one embodiment, the sample is obtained from a mammal, such as a human.

In one embodiment, the sample is an environmental sample. Preferably, the environmental sample is air or water.

In certain embodiments, the sample is, or is derived from, either a composition for immunotherapy or a diagnostic composition. In an embodiment, the method of this aspect is performed to batch standardize the pharmaceutical composition or the diagnostic composition.

In one embodiment, the sample comprises one or a plurality of other grass pollen-derived allergens in addition to said allergen.

In one embodiment, the method of this aspect is for determining a relative or absolute amount of the allergen in the sample

In a third aspect, the invention provides a method of preventing or treating sensitivity to a Johnson grass pollen allergen, or an allergen immunologically cross-reactive with a Johnson grass pollen allergen, in a subject, including the step of administering to said subject a composition comprising a therapeutically effective amount of a Johnson grass pollen allergen or an antibody thereto.

In one embodiment, the subject is a human.

In another embodiment, the therapeutically effective amount of the Johnson grass pollen allergen is administered subcutaneously.

In a further embodiment, the therapeutically effective amount of the Johnson grass pollen allergen is administered sublingually.

Suitably, according to the first, second and third aspects, the Johnson grass pollen allergen is or comprises an isolated allergenic protein.

In particular embodiments, the isolated allergenic protein comprises, consists of or consists essentially of an amino acid sequence set forth in SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48 or SEQ ID NO: 49.

These aspects also include fragments, variants and derivatives of said isolated protein.

In a fourth aspect, the invention provides an isolated protein which comprises, consists of, or consists essentially of an amino acid sequence set forth in SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42 or SEQ ID NO: 43.

This aspect also includes fragments, variants and derivatives of said isolated protein.

In a fifth aspect, the invention provides an antibody or antibody fragment which binds and/or is raised against the isolated protein of the fourth aspect.

The antibody may be a monoclonal antibody or a polyclonal antibody.

In another embodiment, the antibody is a recombinant antibody or antibody fragment.

In a sixth aspect, the invention provides a composition comprising an isolated protein, fragment, variant or derivative, wherein the isolated protein comprises an amino acid sequence according to any one of SEQ ID NOs:1-49 or an antibody that binds or is raised against said isolated protein, fragment, variant or derivative.

Preferably, the antibody or antibody fragment is according to the fifth aspect.

In one embodiment, the composition further comprises one or more additional environmental allergens.

In particular embodiments, the composition further comprises one or more grass pollen allergens from Bahia grass (Paspalum notatum), Bermuda grass (Cynodon dactyln) and/or Ryegrass (Lolium perenne), or one or more antibodies thereto.

In one embodiment, the composition further comprises one or more pharmaceutically acceptable carriers, diluents or excipients.

In another embodiment, the composition is a diagnostic composition.

In a seventh aspect, the invention provides an isolated nucleic acid comprising a nucleotide sequence which encodes, or is complementary to a nucleotide sequence which encodes, the isolated protein of the fourth aspect.

In particular embodiments, the isolated nucleic acid comprises, consists of or consists essentially of a nucleotide sequence set forth in SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88 or SEQ ID NO: 89.

This aspect also includes fragments, variants and derivatives of said isolated nucleic acid.

In an eighth aspect, the invention provides a genetic construct comprising: (i) the isolated nucleic acid of the seventh aspect; or (ii) an isolated nucleic acid comprising a nucleotide sequence complementary thereto; operably linked or connected to one or more regulatory sequences in an expression vector.

In a ninth aspect, the invention provides a host cell transformed with a nucleic acid molecule of the seventh aspect or the genetic construct of eighth aspect.

In a tenth aspect, the invention provides a method of producing the recombinant protein of the fourth aspect, comprising; (i) culturing the previously transformed host cell of the ninth aspect; and (ii) isolating said protein from said host cell cultured in step (i).

In an eleventh aspect, the invention provides a diagnostic and/or screening kit comprising: (i) one or more of the isolated proteins of the aforementioned aspects and/or one or more antibodies that bind or are raised against the proteins; and (ii) instructions for use.

In one embodiment, the kit further comprises one or more additional environmental allergens or antibodies raised against one or more additional environmental allergens.

In a twelfth aspect, the invention provides a method of determining the amino acid sequence of a grass pollen allergen, including the steps of: (i) preparing cDNA from RNA extracted from a grass pollen; (ii) determining the nucleotide sequence of said cDNA library; (iii) isolating allergenic proteins or fragments thereof from the corresponding grass pollen in (i); (iv) determining the amino acid sequence of the isolated allergen proteins or fragments thereof from (iii).

Preferably, the method further comprises extracting RNA from a grass pollen and preparing an RNA fragment library from said RNA.

Preferably, the method further includes the step of confirming the amino acid sequence of (iii) by aligning and comparing the predicted peptide sequence encoding the nucleotide sequence of (ii) with the amino acid sequence of (iii).

Throughout this specification, unless the context requires otherwise, the words “comprise”, “comprises” and “comprising” will be understood to imply the inclusion of a stated integer or group of integers but not the exclusion of any other integer or group of integers.

### BRIEF DESCRIPTION OF THE FIGURES

FIG. 1. Allergic sensitivity to JGP allergens. (A) Skin prick test of non-atopic subjects (n=19), patients with grass pollen allergy (n=48), and allergies other than grass pollen (n=24). (individual data with median and IQR, cut-off line at 3 mm). (B) Serum IgE immunoblots of JGP. (Molecular weights in kDa, arrows designate major allergen components).

FIG. 2. Identification of JGP allergenic components. (A) 2D gel electrophoresis of JGP stained with Coomassie Blue. 2D IgE immunoblots of JGP probed with (B) a JGP-allergic patient serum pool (patients from FIG. 1B, arrows mark IgE-reactive components; replica immunoblot with pool of non-atopic sera from FIG. 1B showed no IgE reactivity, not shown), and (C, D) specific mAb (Sor h 1 and Sor h 13 isoforms marked).

FIG. 3. Serum IgE reactivity with Sor h 1 and Sor h 13. IgE reactivity with each allergen normalized to nonatopic donors. (Box; median and IQR, and whiskers; 10th-90th percentiles). The cut off of three SD above the mean of 23 non-atopic subjects in (A) for JGP (OD=0.410) and Sor h 1 (OD=0.229) and (B) for JGP (OD=0.371) and Sor h 13 (OD=0.384). P values by Mann Whitney U test. Correlation between IgE reactivity with JGP and Sor h 1 (C) and Sor h 13 (D). (Spearman's correlation and CI given). (E) Frequency of IgE reactivity with JGP, Sor h 1 and Sor h 13. (F) Purified JGP allergens stained with Coomassie blue and immunoblotted with mAb specific to group 1 and group 13 allergens (marked).

FIG. 4. Johnson grass pollen transcriptome assembly analysis. (A) Output results for raw and clean reads of Johnson grass pollen transcriptome sequencing. (B) Output for assembly quality of the transcriptome. (C) Unigenes were annotated with the databases of NR, NT, SwissProt, KEGG, COG and GO.

FIG. 5. Non-Redundant database classification of the Johnson grass pollen transcriptome. (A) BLAST E-value distribution; (B) Identity distribution; and (C) Species distribution of homologous sequence matches. NR database (http://www.ncbi.nlm.nih.gov/).

FIG. 6. IgE reactivity with Sor h 1 as non-normalized data. Serum IgE responses shown as Optical Density Units. Cut off for positive test response of three standard deviations above the mean of 19 non-atopic donors for each allergen preparation is marked. P value by Wilcoxon signed rank test for paired data.

FIG. 7. Alignment of group 1 allergen sequences including Sor h 1.01A (CL153) and Sor h 1.02B (UG493-492) to other known grass pollen group 1 allergens. Allergens cluster according to subfamily. Sequences of subtropical grass families Panicoideae (maize pollen; Zea m 1, Bahia grass pollen; Pas n 1, and Johnson grass Sor h 1), Ehrhartoideae (rice Ory s 1) and Chloridoideae (Bermuda grass; Cyn d 1) align in separate clades distant to the Pooideae temperate grass pollens (Ryegrass; Lol p 1, Timothy grass pollen; Phl p 1, Brachypodium sp; Bra di 1, Bra sy 1, Canary grass; Pha a 1, Orchard grass; Dac g 1, Rye; Sec c 1, Kentucky Blue grass; Poa p 1, Velvet grass; Hol l 1, meadow ryegrass; Fes p and Barley pollen; Hor v 13)

FIG. 8. Alignment of group 13 allergen sequences showing Panicoideae sequences (maize pollen; Zea m 13, Bahia grass pollen; Pas n 13 and Johnson grass pollen; Sor h 13) in separate clade to Pooideae group 13 allergens (Timothy grass pollen; Phl p 13, Brachypodium distachyon; Bra di 13 and Barley pollen; Hor v 13).

FIG. 9. TCoffee alignment of Sor h 23 (CL2015.1) predicted peptide with group 5 allergens reveals a shared domain not previously identified in any subtropical grass pollen. Phl; Phleum pratensis timothy grass pollen Phl p 5, Poa; Poa pratense Poa p 5, Dac; orchard grass Dactylis glomerata Dac g 5, Lol; Lolium perenne ryegrass Lol p 5, Cyn; Cynodon dactylon Cyn d 23. Bad avg good colour scale represents degree of similarity.

FIG. 10. Coverage of observed peptide spectra of IgE—reactive protein spots excised from 2D gels for spots for CL 153.1 Spot 1 (pI 6.8/30 kDa, blue), 2 (pI 7.1/30 kDa, yellow) and 3 (pI 10.5/30 kDa, green). Spot 1 shows 78% coverage of amino acids across the mature peptide sequence.

FIG. 11. Alignment of Sor h 1.02B to closest match in BLAST search: XP_002467539 (Sorghum bicolor). Alignment of Sor h 1.02B with the S. bicolor XP_002467539 verifies the validity of the sequences as a complete coding transcript arising from a single gene locus.

FIG. 12. Alignment of Sor h 1.0A (CL153.1) and Sor h 1.02B (UG493-492) peptides. The relatively lower than expected amino acid percentage identity (57%) and similarity (73%) between CL153 and UG493-492 suggests these transcripts are encoded by separate gene loci. The genetic loci encode beta expansin allergens Sor h 1.01A and Sor h 1.02B with different biochemical characteristics. These are likely to confer different immunological properties and may elicit distinct B and T cell responses from patients with grass pollen allergy. These separate allergen isoforms are likely to contain some shared as well as distinct B and T cell epitopes.

FIG. 13. Alignment of CL2015.1 (Sor h 23) to closest match in BLAST search; hypothetical protein XP 002446575.1 (Sorghum bicolor).

FIG. 14. Alignment of CL2015.1 (Sor h 23) to pollen allergen Cyn d 23 (Cynodon dactylon).

FIG. 15. Coverage of observed peptide spectra of IgE-reactive protein spots excised from 2D gels for spot four with CL2015.1 (Sor h 23). The spectra observed cover 66% of the CL2015.1 sequence verifying the presence of this sequence as that encoding the IgE reactive spot.

FIG. 16. Coverage of observed peptide spectra of IgE-reactive protein spots excised from 2D gels for spot five with CL2015.1 (Sor h 23). The spectra observed cover 73% of the CL2015.1 sequence verifying the presence of this sequence as that encoding the IgE reactive spot.

FIG. 17. Alignment of UG388 encoding spot 6 with closest match identified by database search. This sequence has no history of association with allergy.

FIG. 18. Alignment of CL1122.1 (Sor h 2.01) with sequence of maize with homology to group 2 allergen of timothy grass pollen.

FIG. 19. Alignment of CL1122.2 (Sor h 2.03) with closest database match verifying its sequence from S. bicolor.

FIG. 20. Alignment of CL1122.2 (Sor h 2.03) with group 3 pollen allergen (Zea mays).

FIG. 21. Alignment of CL1122.2 (Sor h 2.03) with putative group 3 pollen allergen (Oryza sativa Japonica Group).

FIG. 22. Alignment of CL 1695 (Sor h 2.02) peptide with closest database match of S. bicolor verifying its existence.

FIG. 23. Alignment of CL 1695 (Sor h 2.02) peptide with group 2 homolog in maize.

FIG. 24. Coverage of observed peptide spectra of spots 7 and 8 with predicted peptides of closest matches to CL1122.1 (Sor h 2.01) and CL 1695 (Sor h 2.02). Data confirms presence of these IgE reactive allergens within the proteome and transcriptome of JGP.

FIG. 25. Sequence alignment of CL1737.1 (Sor h 13.01A) and CL1737.2 (Sor h 13.01B).

FIG. 26. Sequence identity percentages for the closest protein and Pas n 13 allergen matches of CL737.1 (Sor h 13.01A) and CL1737.2 (Sor h 13.01B).

FIG. 27. Coverage of peptide spectra for mass spec of purified Sor h 13 A and Sor h 13 B aligned to CL1737.1 and CL1737.2. These are two previously undescribed unique transcripts that encode isoforms of Sor h 13. Both are represented within peptides in the proteome of JGP.

FIG. 28. Nucleotide sequence for Sor h 1.028 transcript. Both coding and untranslated sequence is provided. Nucleotide sequences and predicted peptide sequence for concatenation of Unigene 493 reverse complement to Unigene 492 minus the eight nucleotide overlap are provided. ATG start and Stop codons shown in yellow and red respectively. Signal peptide has been underlined.

FIG. 29. Nucleotide sequence for Sor h 13.01A (CL1737.1) transcript. Both coding and untranslated sequence is provided. Translated region and predicted amino acid sequence are given. Signal peptide junction shown by arrow.

FIG. 30. Nucleotide sequence for Sor h 13.01B (CL1737.2) transcript. Both coding and untranslated sequence is provided. Translated region and predicted amino acid sequence are given. Signal peptide junction shown by arrow.

FIG. 31. Nucleotide sequence for CL110 transcript. Both coding and untranslated sequence is provided. Translated region and predicted amino acid sequence are given.

FIG. 32. Nucleotide sequence for CL1152 transcript. Both coding and untranslated sequence is provided. Translated region and predicted amino acid sequence are given.

FIG. 33. Nucleotide sequence for CL1715 transcript. Both coding and untranslated sequence is provided. Translated region and predicted amino acid sequence are given.

FIG. 34. Nucleotide sequence for CL1444 transcript. Both coding and untranslated sequence is provided. Translated region and predicted amino acid sequence are given.

FIG. 35. Nucleotide sequence for CL1754 transcript. Both coding and untranslated sequence is provided. Translated region and predicted amino acid sequence ar given.

FIG. 36. Nucleotide sequence for CL200 transcript. Both coding and untranslated sequence is provided. Translated region and predicted amino acid sequence are given.

FIG. 37. Nucleotide sequence for CL2015.2 transcript. Both coding and untranslated sequence is provided. Translated region and predicted amino acid sequence are given.

FIG. 38. Nucleotide sequence for CL2052 transcript. Both coding and untranslated sequence is provided. Translated region and predicted amino acid sequence are given.

FIG. 39. Nucleotide sequence for CL248 transcript. Both coding and untranslated sequence is provided. Translated region and predicted amino acid sequence are given.

FIG. 40. Nucleotide sequence for CL70 transcript. Both coding and untranslated sequence is provided. Translated region and predicted amino acid sequence are given.

FIG. 41. Nucleotide sequence for CL830 transcript. Both coding and untranslated sequence is provided. Translated region and predicted amino acid sequence are given.

FIG. 42. Nucleotide sequence for CL962 transcript. Both coding and untranslated sequence is provided. Translated region and predicted amino acid sequence are given.

FIG. 43. Nucleotide sequence for CL986 transcript. Both coding and untranslated sequence is provided. Translated region and predicted amino acid sequence are given.

FIG. 44. Nucleotide sequence for UG1043 transcript. Both coding and untranslated sequence is provided. Translated region and predicted amino acid sequence are given.

FIG. 45. Nucleotide sequence for UG11756 transcript. Both coding and untranslated sequence is provided. Translated region and predicted amino acid sequence are given.

FIG. 46. Nucleotide sequence for UG1334 transcript. Both coding and untranslated sequence is provided. Translated region and predicted amino acid sequence are given.

FIG. 47. Nucleotide sequence for UG1403 transcript. Both coding and untranslated sequence is provided. Translated region and predicted amino acid sequence are given.

FIG. 48. Nucleotide sequence for UG2745 transcript. Both coding and untranslated sequence is provided. Translated region and predicted amino acid sequence are given.

FIG. 49. Nucleotide sequence for UG308 transcript. Both coding and untranslated sequence is provided. Translated region and predicted amino acid sequence are given.

FIG. 50. Nucleotide sequence for UG332 transcript. Both coding and untranslated sequence is provided. Translated region and predicted amino acid sequence are given.

FIG. 51. Nucleotide sequence for UG335 transcript. Both coding and untranslated sequence is provided. Translated region and predicted amino acid sequence are given.

FIG. 52. Nucleotide sequence for UG342 transcript. Both coding and untranslated sequence is provided. Translated region and predicted amino acid sequence are given.

FIG. 53. Nucleotide sequence for UG397 transcript. Both coding and untranslated sequence is provided. Translated region and predicted amino acid sequence are given.

FIG. 54. Nucleotide sequence for UG41 transcript. Both coding and untranslated sequence is provided. Translated region and predicted amino acid sequence are given.

FIG. 55. Nucleotide sequence for UG540 transcript. Both coding and untranslated sequence is provided. Translated region and predicted amino acid sequence are given.

FIG. 56. Nucleotide sequence for UG5446 transcript. Both coding and untranslated sequence is provided. Translated region and predicted amino acid sequence are given.

FIG. 57. Nucleotide sequence for UG551 transcript. Both coding and untranslated sequence is provided. Translated region and predicted amino acid sequence are given.

FIG. 58. Nucleotide sequence for UG552 transcript. Both coding and untranslated sequence is provided. Translated region and predicted amino acid sequence are given.

FIG. 59. Nucleotide sequence for UG578 transcript. Both coding and untranslated sequence is provided. Translated region and predicted amino acid sequence are given.

FIG. 60. Nucleotide sequence for UG6038 transcript. Both coding and untranslated sequence is provided. Translated region and predicted amino acid sequence are given.

FIG. 61. Nucleotide sequence for UG681 transcript. Both coding and untranslated sequence is provided. Translated region and predicted amino acid sequence are given.

FIG. 62. Nucleotide sequence for UG6635 transcript. Both coding and untranslated sequence is provided. Translated region and predicted amino acid sequence are given.

FIG. 63. Nucleotide sequence for UG7876 transcript. Both coding and untranslated sequence is provided. Translated region and predicted amino acid sequence are given.

FIG. 64. Nucleotide sequence for UG808 transcript. Both coding and untranslated sequence is provided. Translated region and predicted amino acid sequence are given.

FIG. 65. Nucleotide sequence for UG832 transcript. Both coding and untranslated sequence is provided. Translated region and predicted amino acid sequence are given.

FIG. 66. Nucleotide sequence for UG8760 transcript. Both coding and untranslated sequence is provided. Translated region and predicted amino acid sequence are given.

FIG. 67. Nucleotide sequence for UG9701 transcript. Both coding and untranslated sequence is provided. Translated region and predicted amino acid sequence are given.

FIG. 68. Amino acid sequence for CL153 (Sor h 1.01A) transcript. Sequence for both the signal peptide (27 amino acids) and the mature peptide (239 amino acids) is provided.

FIG. 69. Nucleotide sequence for Contig1122.1 (Sor h 2.01) transcript. Both coding and untranslated sequence is provided. Translated region and predicted amino acid sequence are given.

FIG. 70. Nucleotide sequence for Contig1695 (Sor h 2.02) transcript. Both coding and untranslated sequence is provided. Translated region and predicted amino acid sequence are given.

FIG. 71. Nucleotide sequence for Contig1122.2 (Sor h 2.03) transcript. Both coding and untranslated sequence is provided. Translated region and predicted amino acid sequence are given.

FIG. 72. Nucleotide sequence for Contig2015.1 (Sor h 23) transcript. Both coding and untranslated sequence is provided. Translated region and predicted amino acid sequence are given.

FIG. 73. Nucleotide sequence for UG388 (spot 6) transcript. Both coding and untranslated sequence is provided. Translated region and predicted amino acid sequence are given.

FIG. 74. Alignment of group 2 allergen sequences including Sor h 2.03. This shows that all of the group 2 allergens of Johnson grass pollen (Sor h 2.01, Sor h 2.02 and Sor h 2.03) align with the group 2 allergens rather than group 3 allergens.

FIG. 75. Alignment of group 23 allergen sequences of subtropical grasses with group 5 allergen sequences of the temperate grasses.

FIG. 76. Concatenation of the sequence of UG492 and UG493. A. Match identified between UG493 to an unidentified sequence XP_002467539.1 (sbjct). B. Match identified between UG492-1 to the same hypothetical protein XP_002467539.1 (sbjct). C. Alignment of Sor h 1.02B, deduced by concatenation of amino acids 1 to 158 of UG493 to amino acids 3 to 109 of UG 492.1, with the S. bicolor sequence XP 002467539.

### BRIEF DESCRIPTION OF THE SEQUENCES

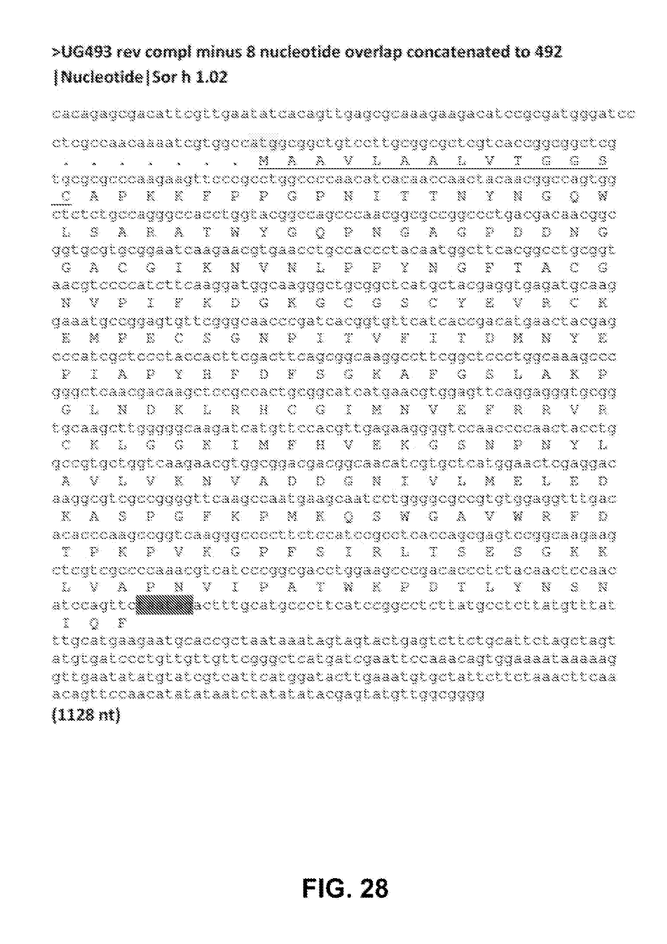

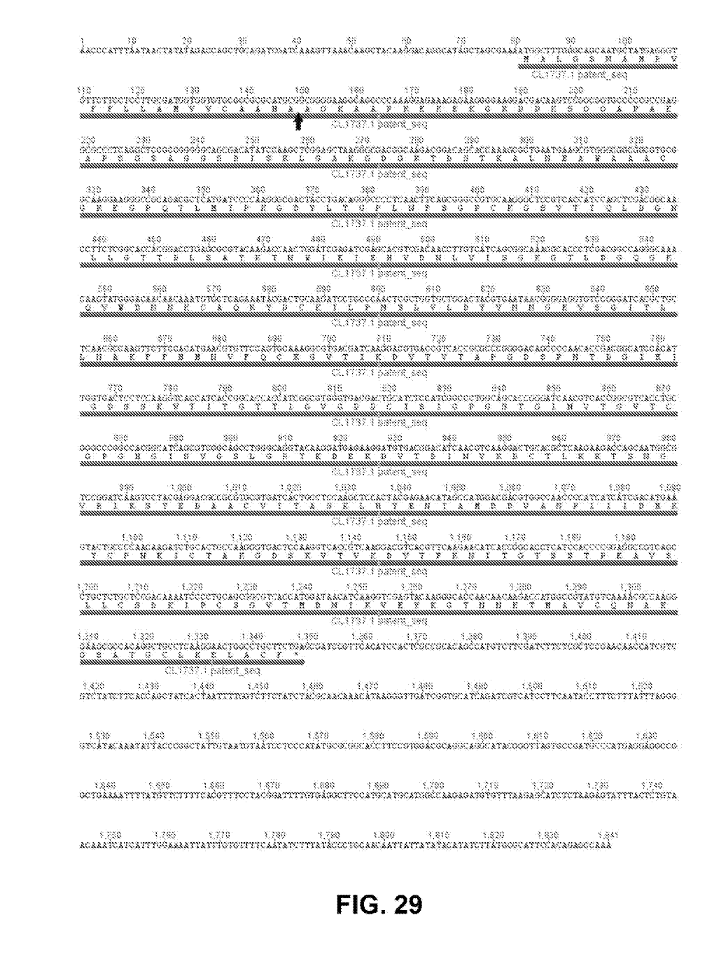

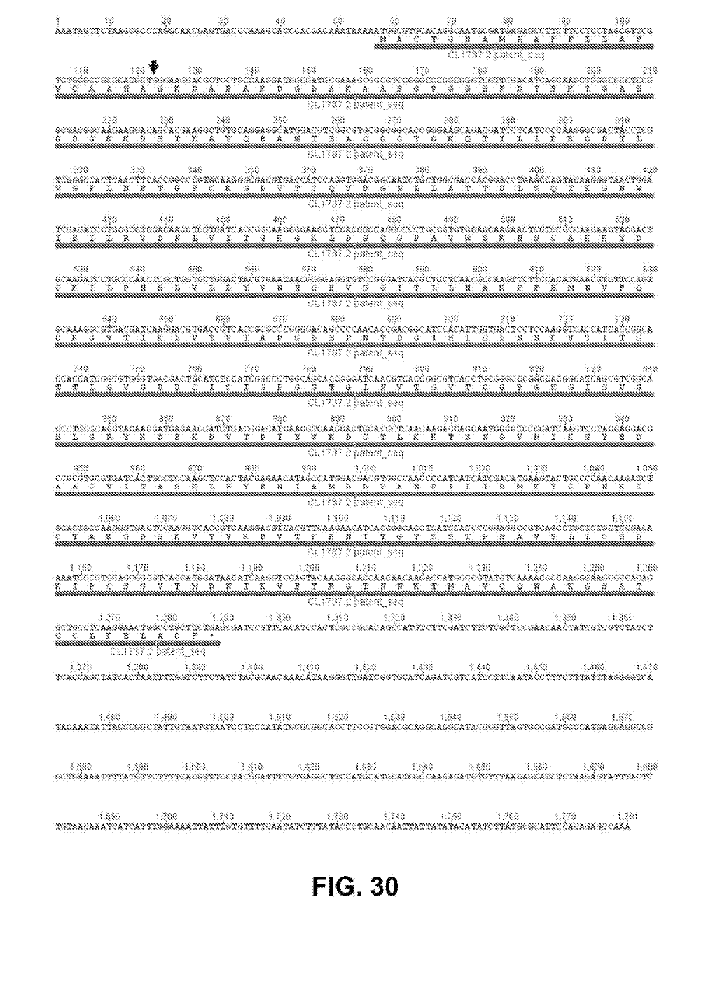

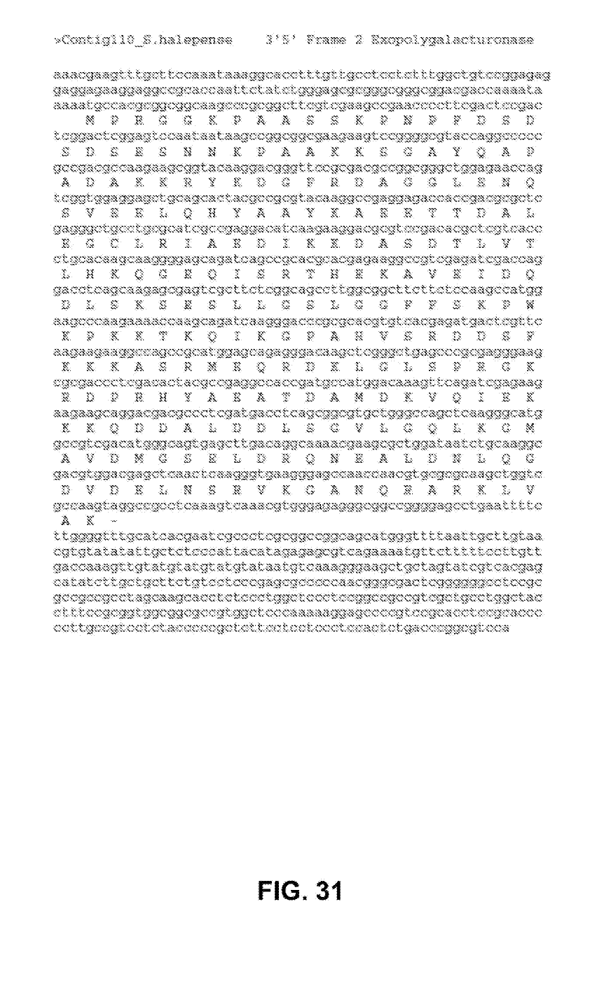

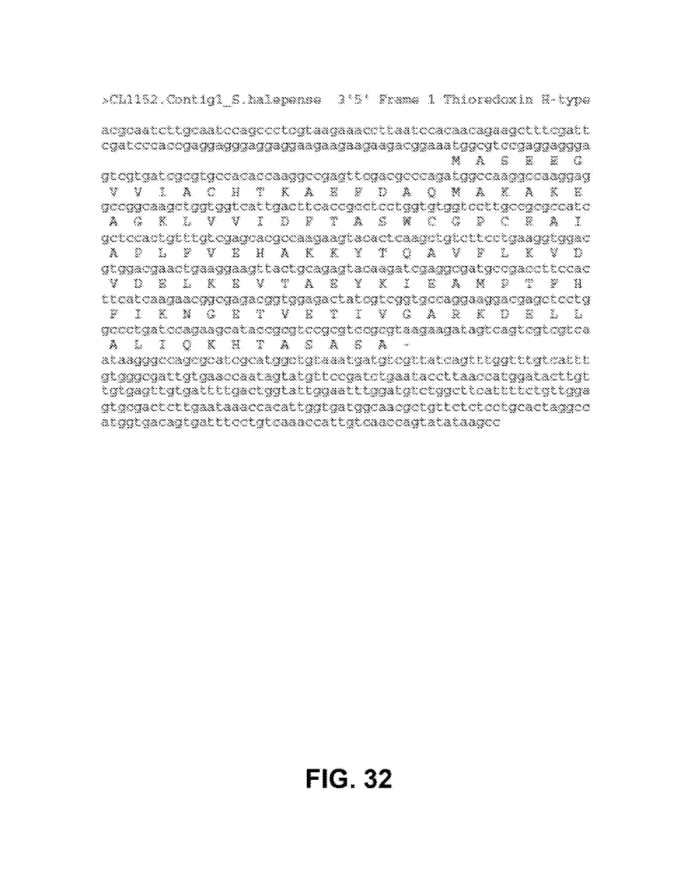

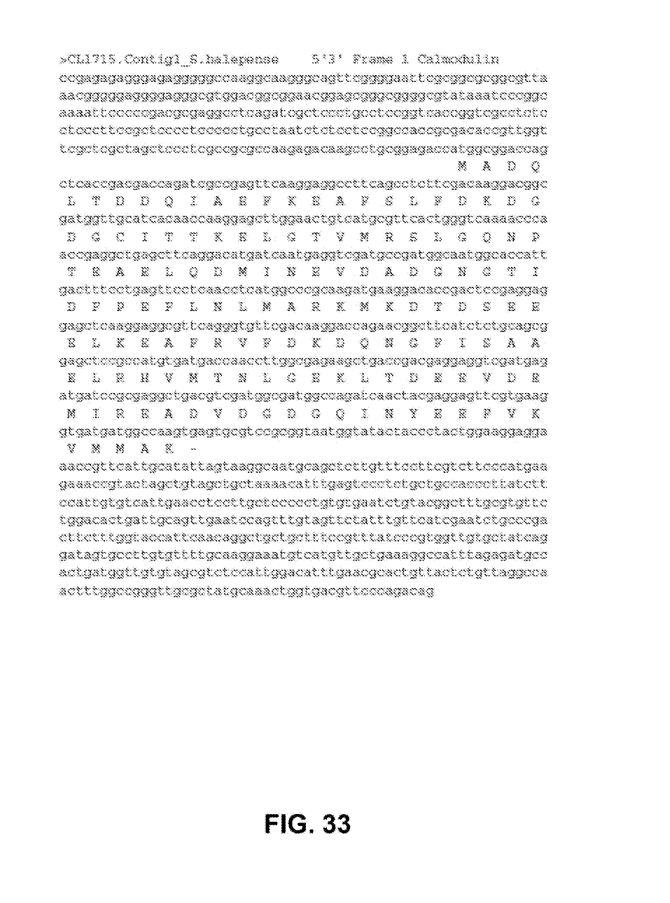

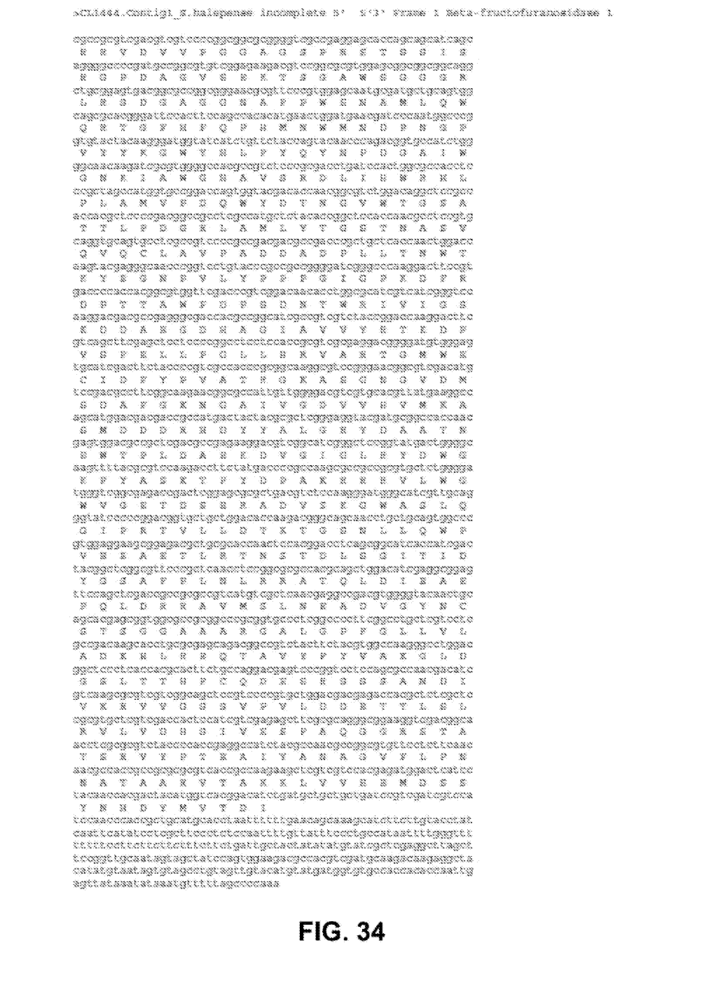

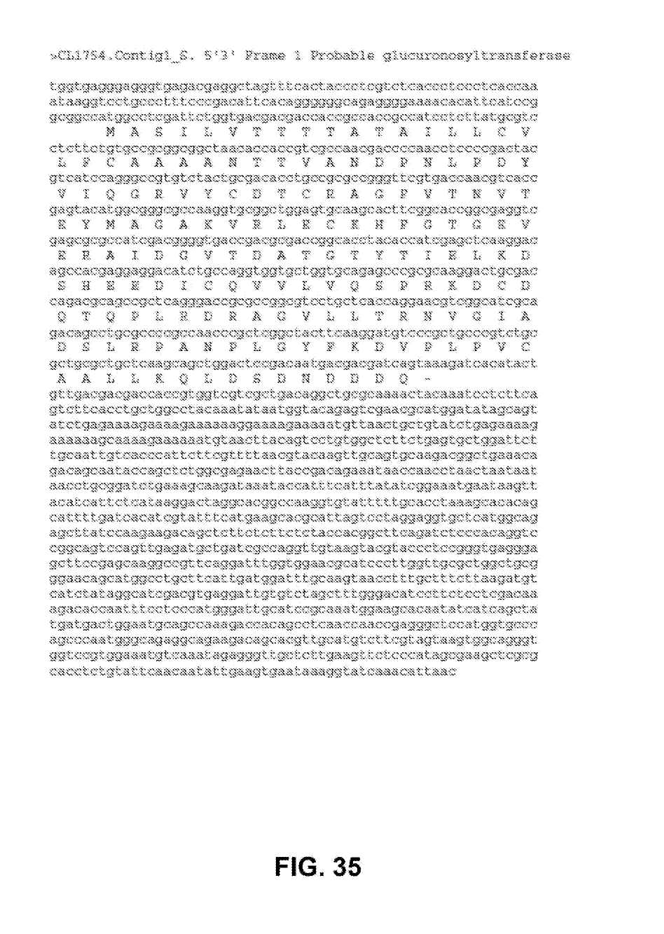

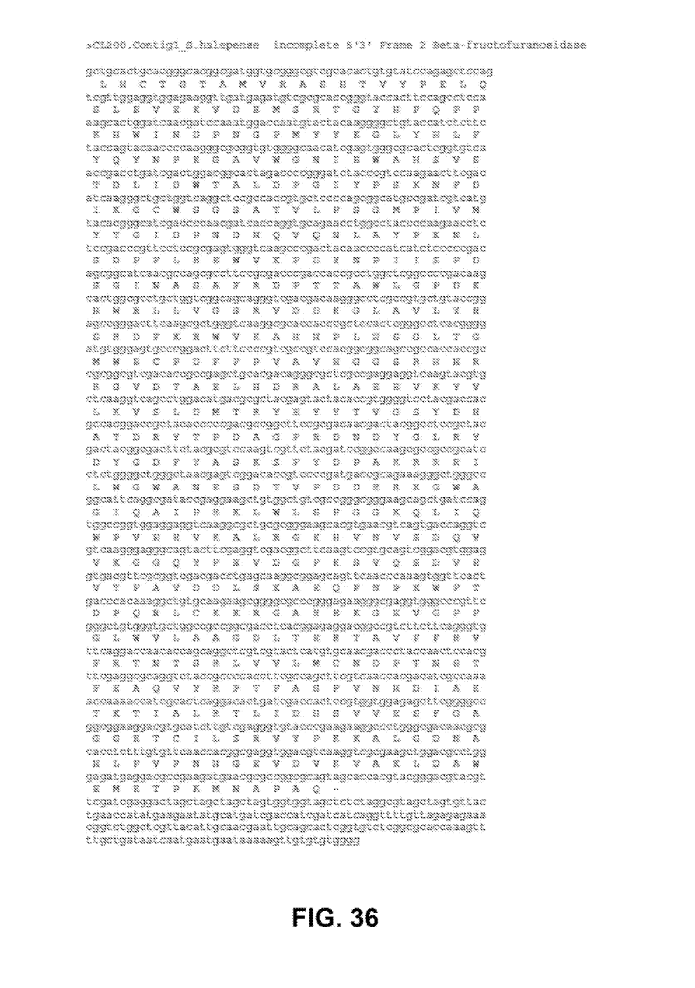

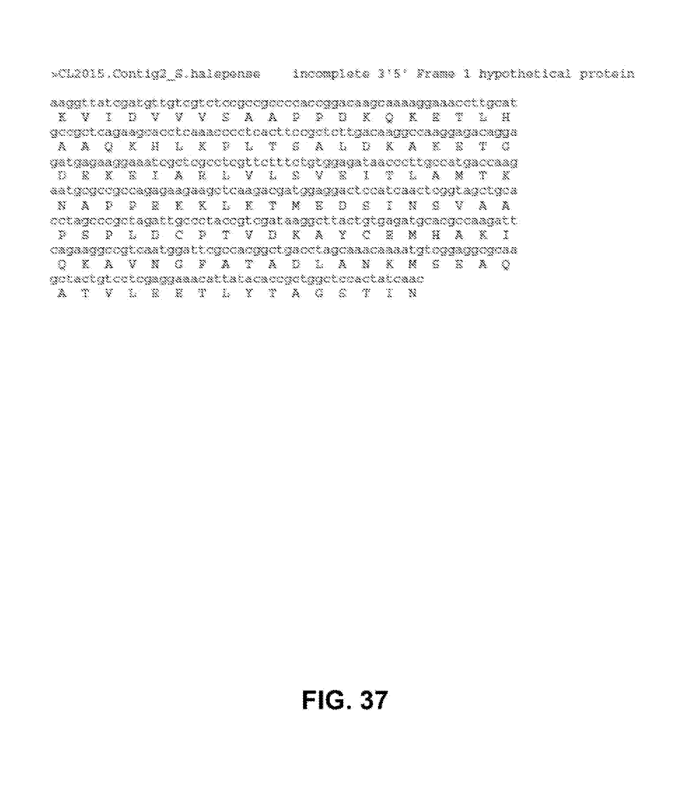

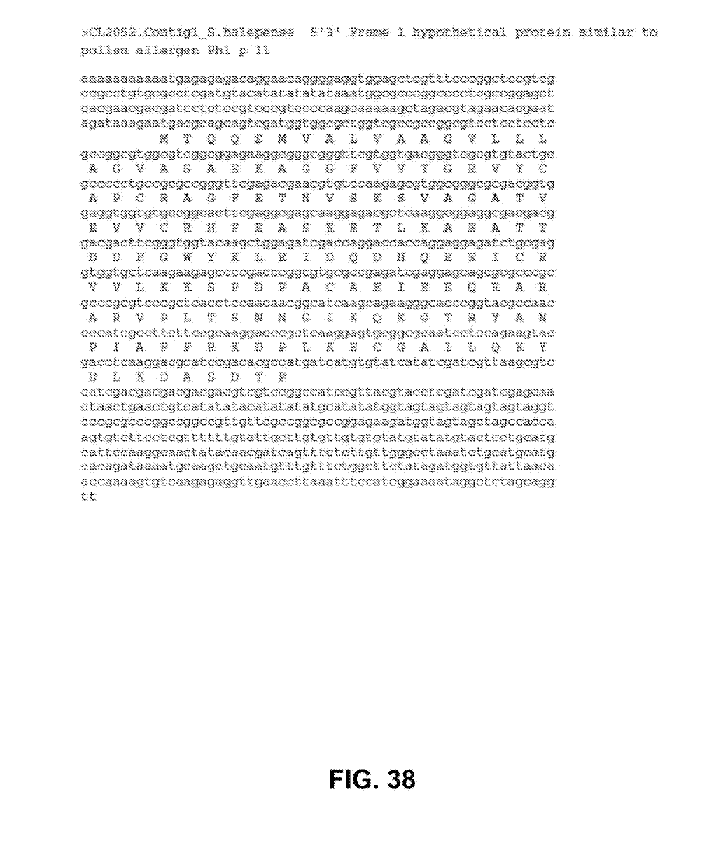

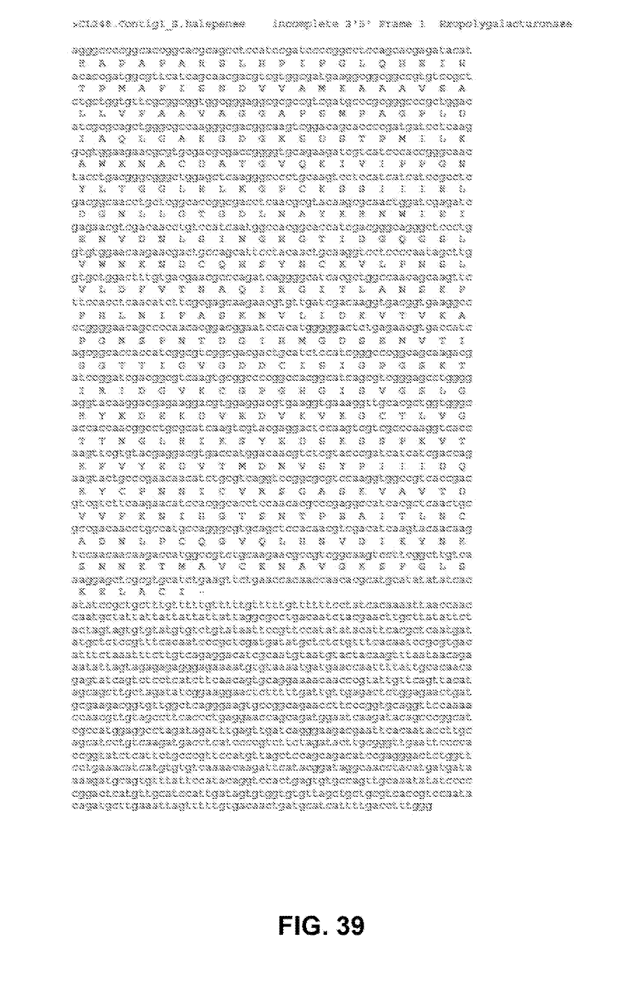

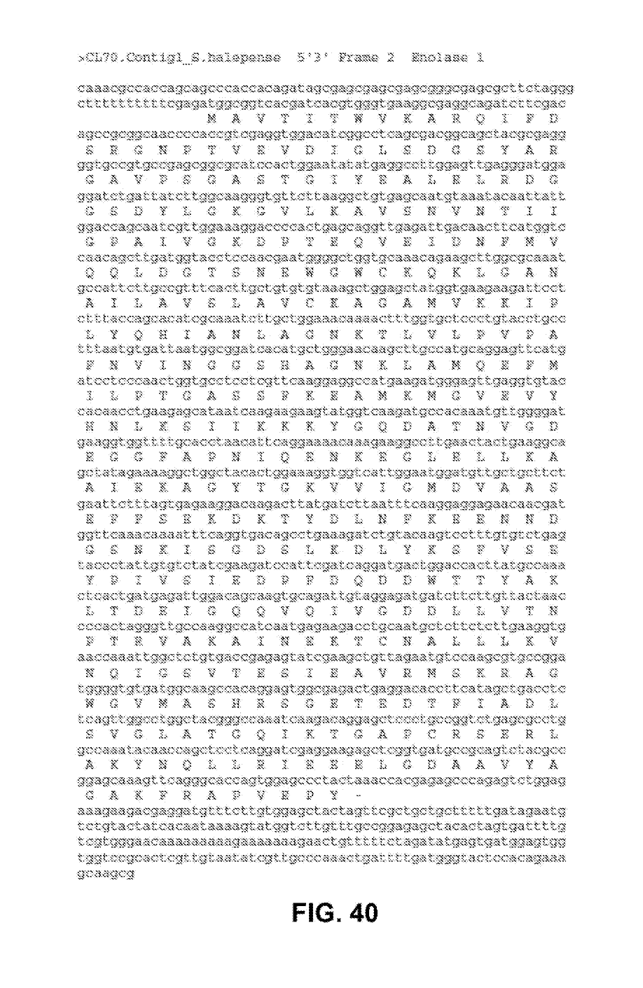

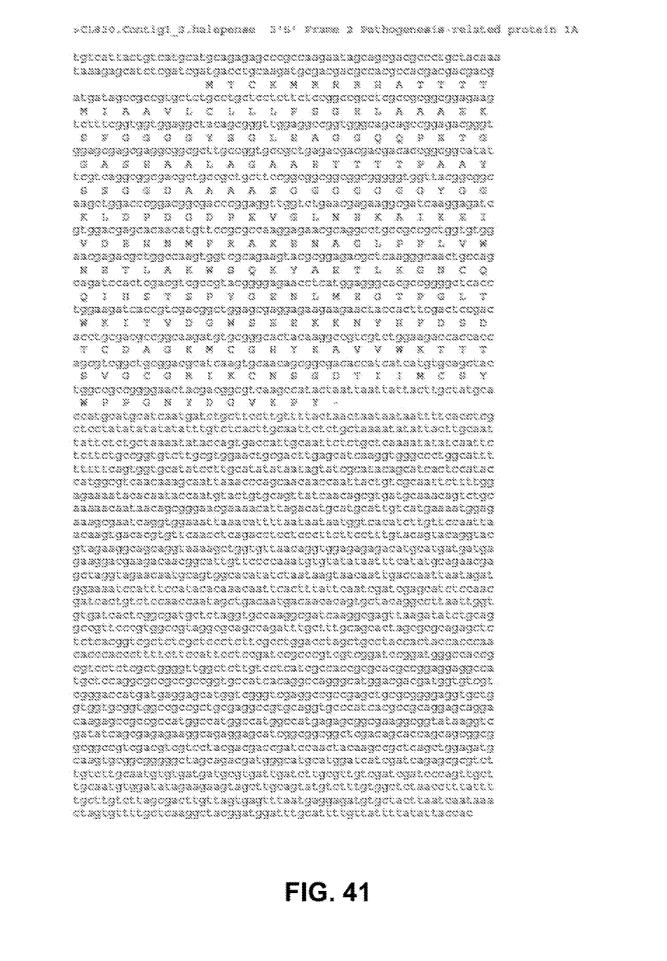

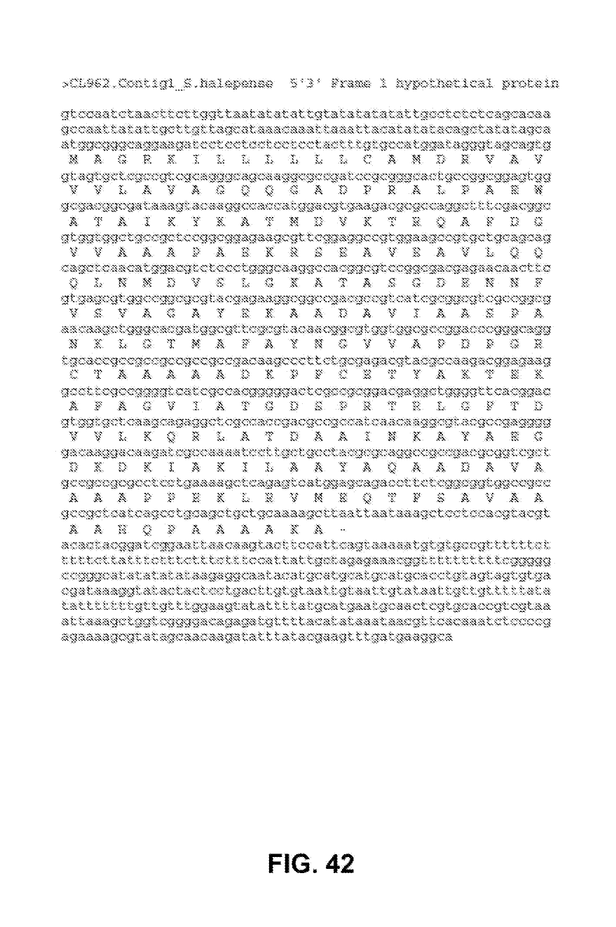

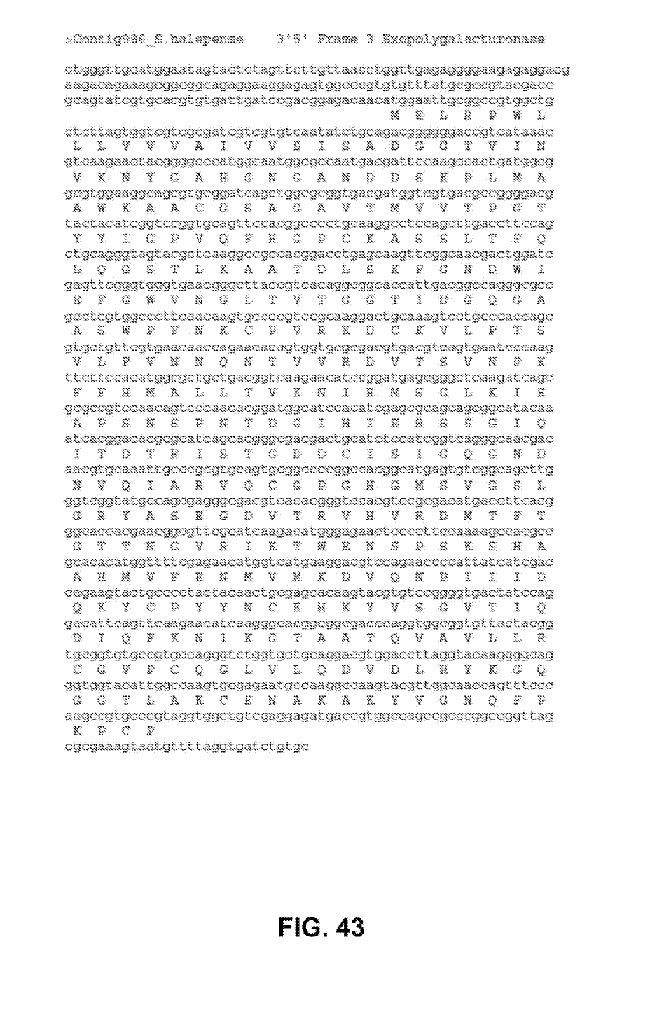

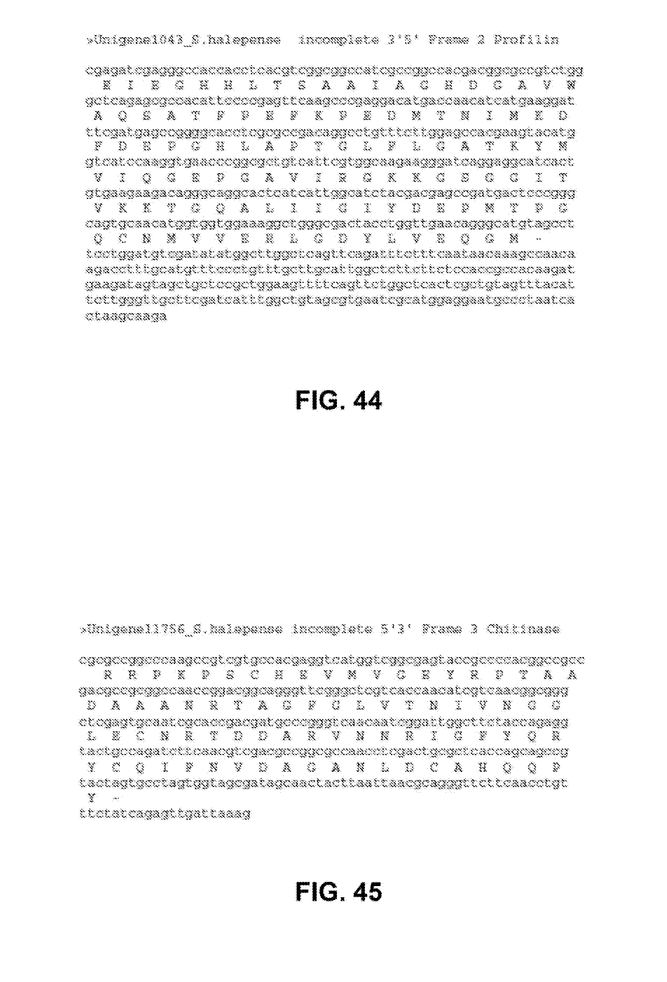

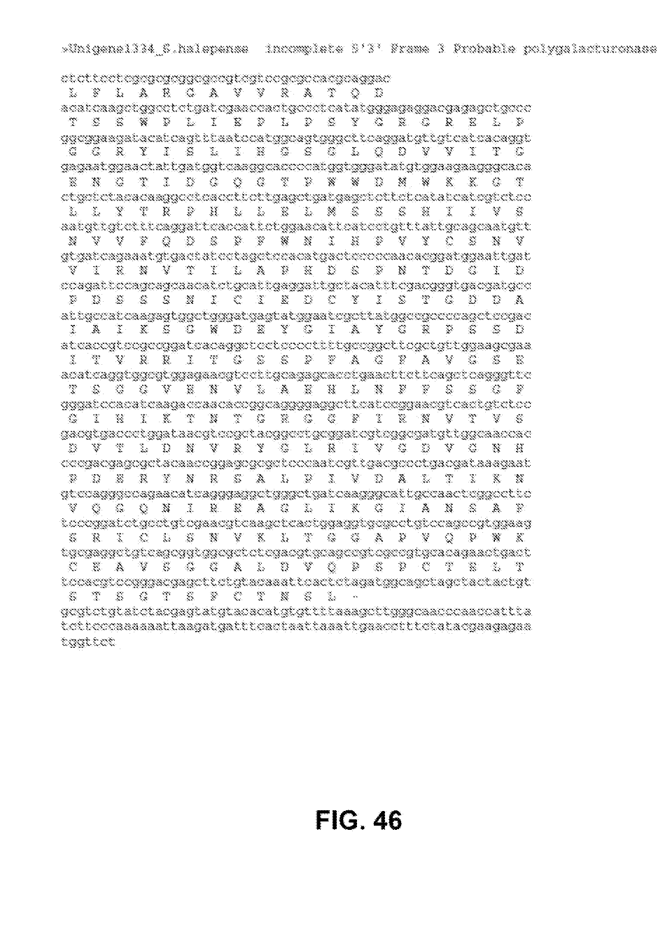

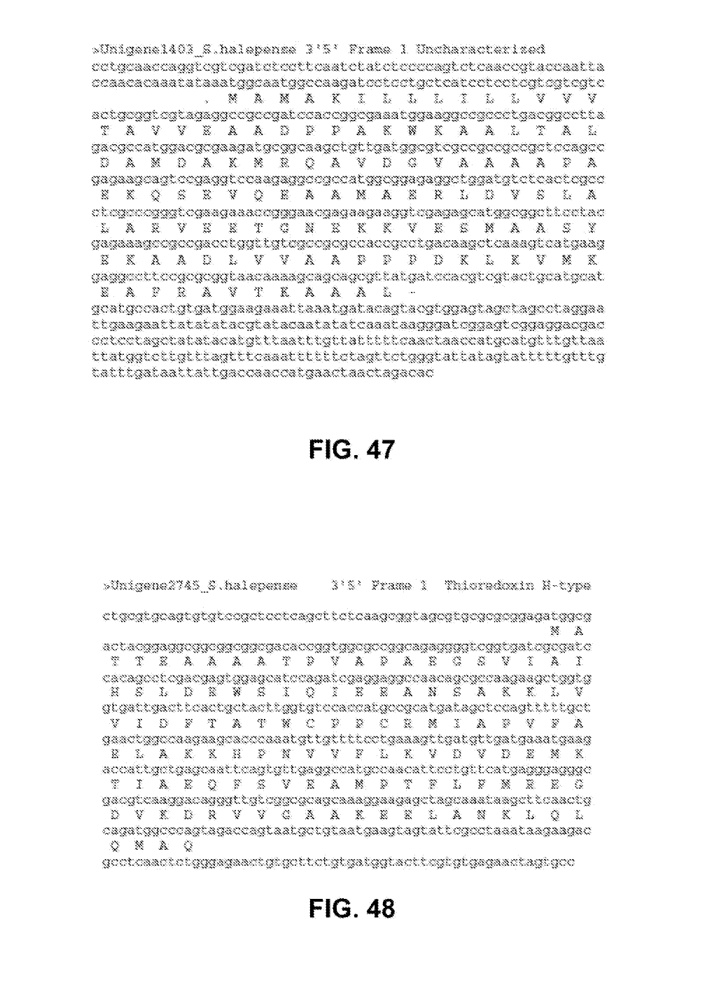

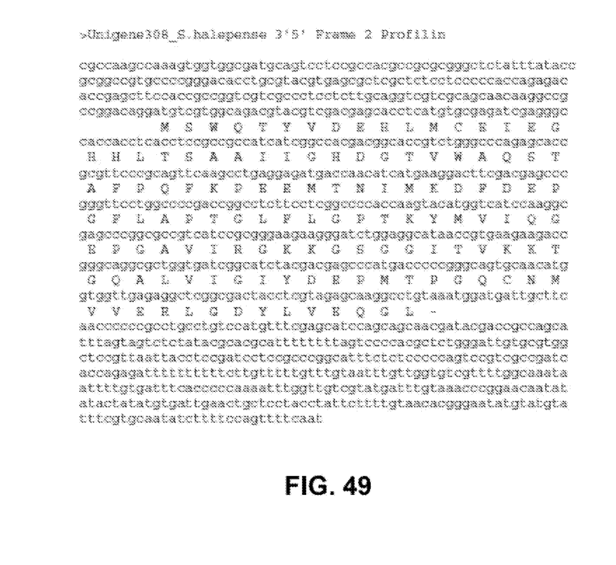

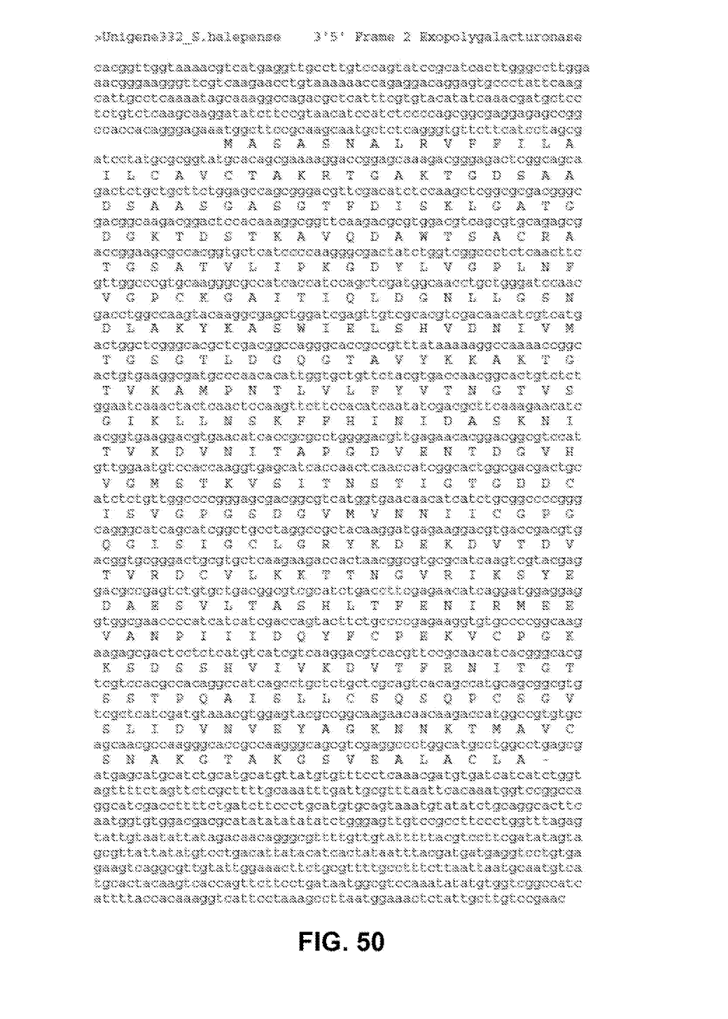

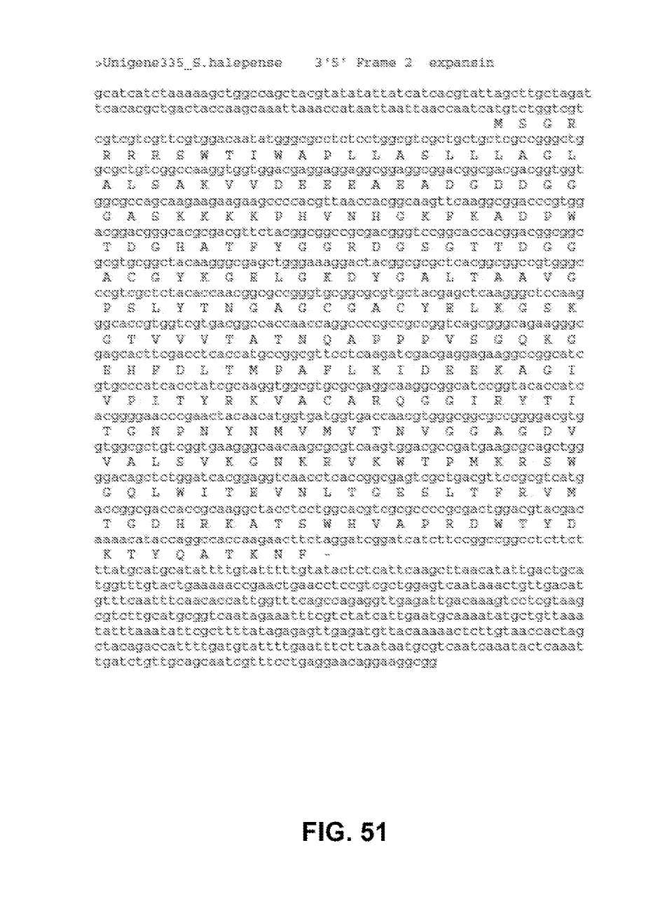

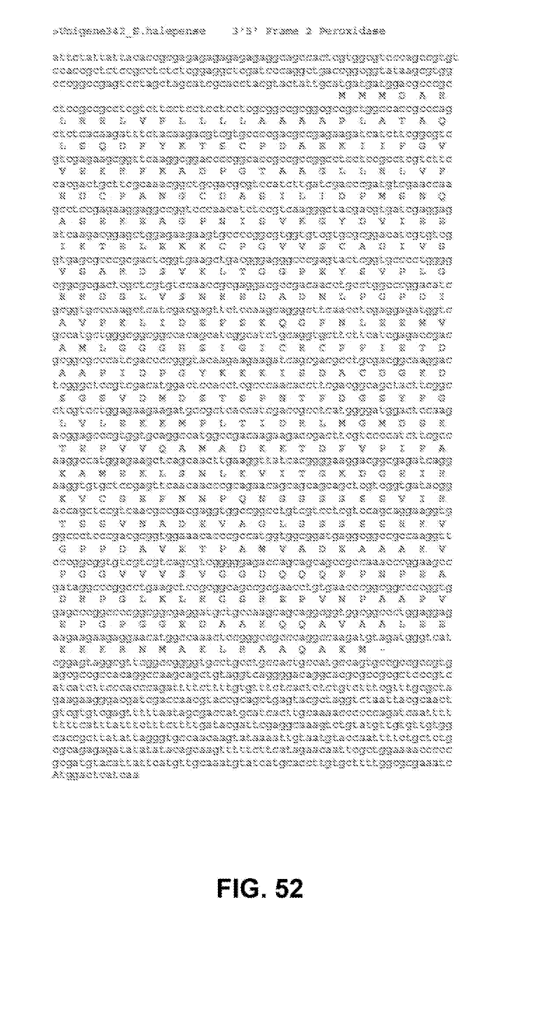

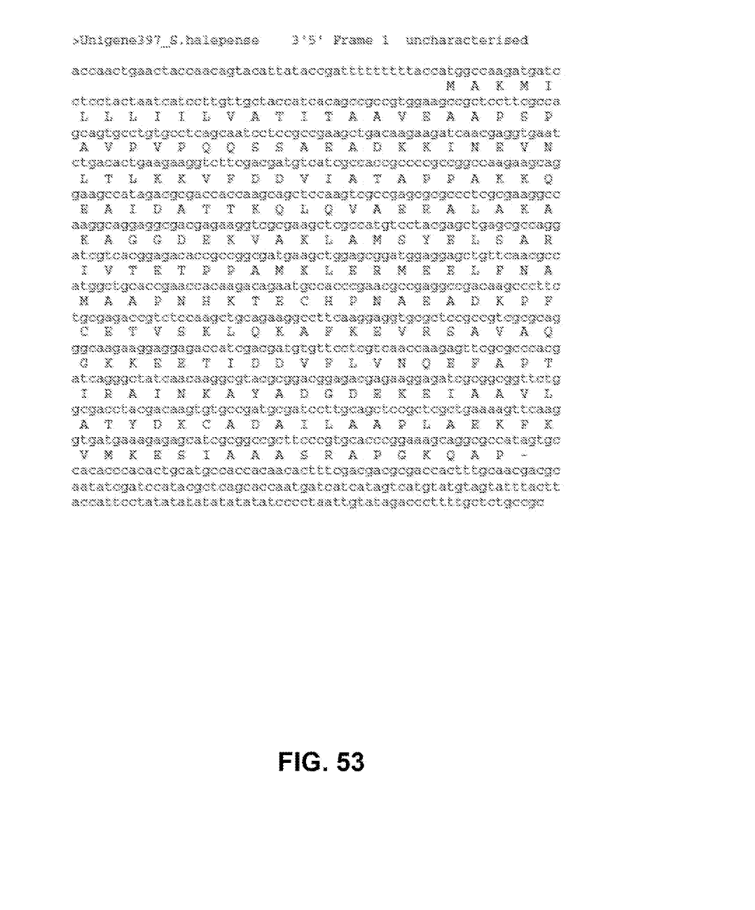

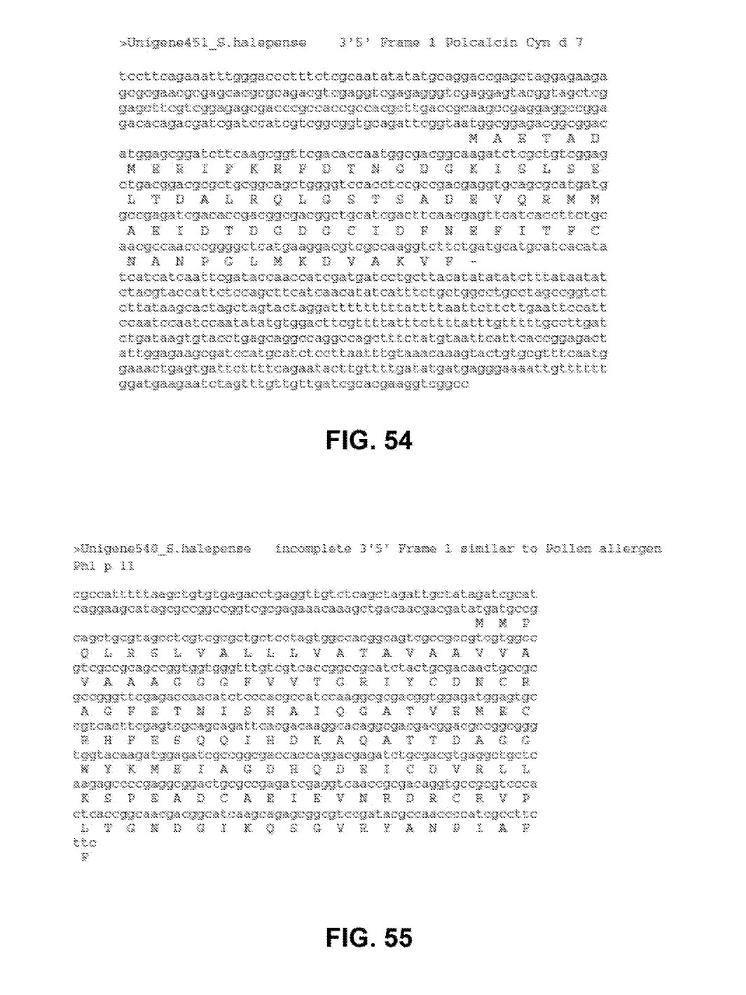

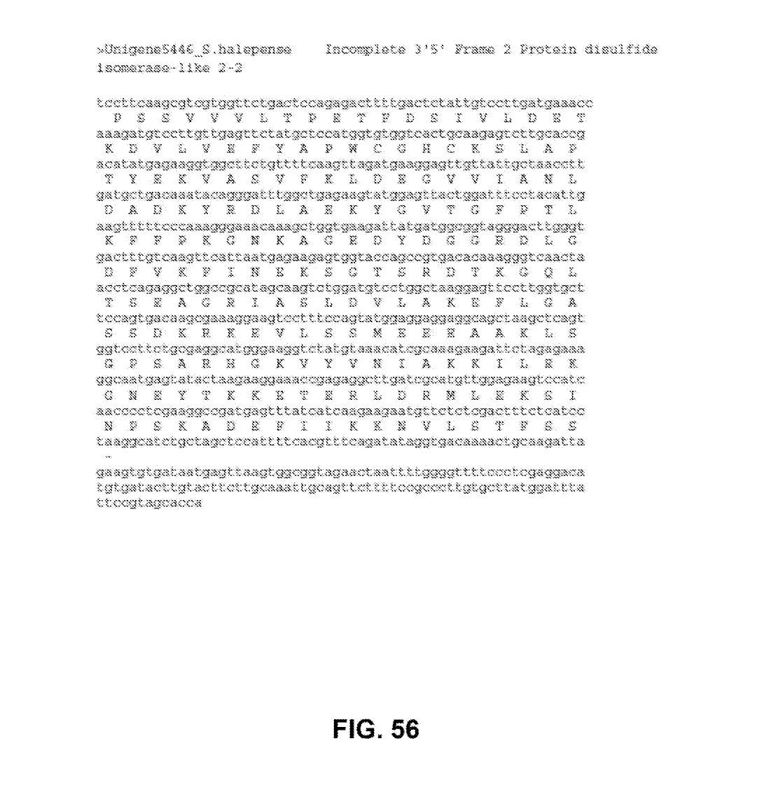

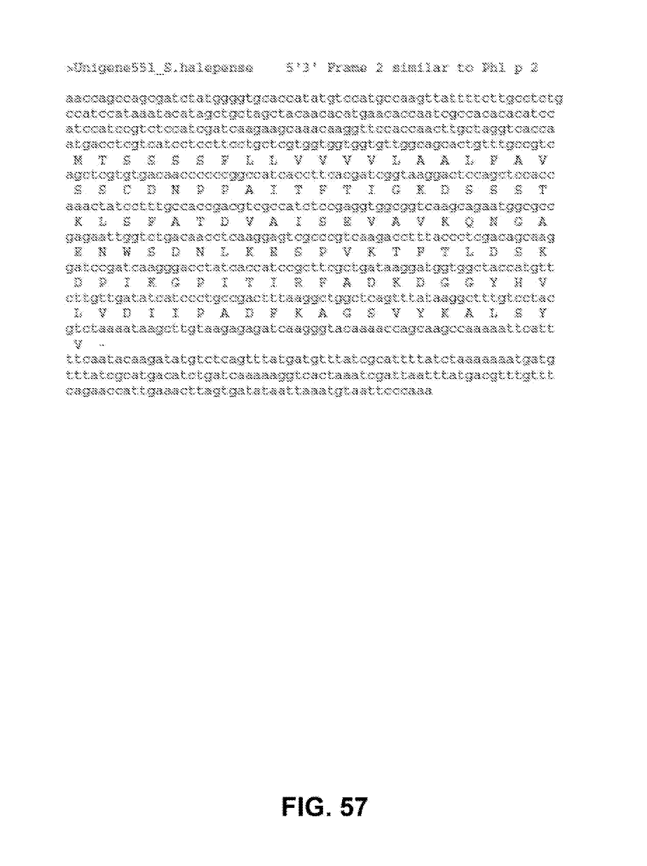

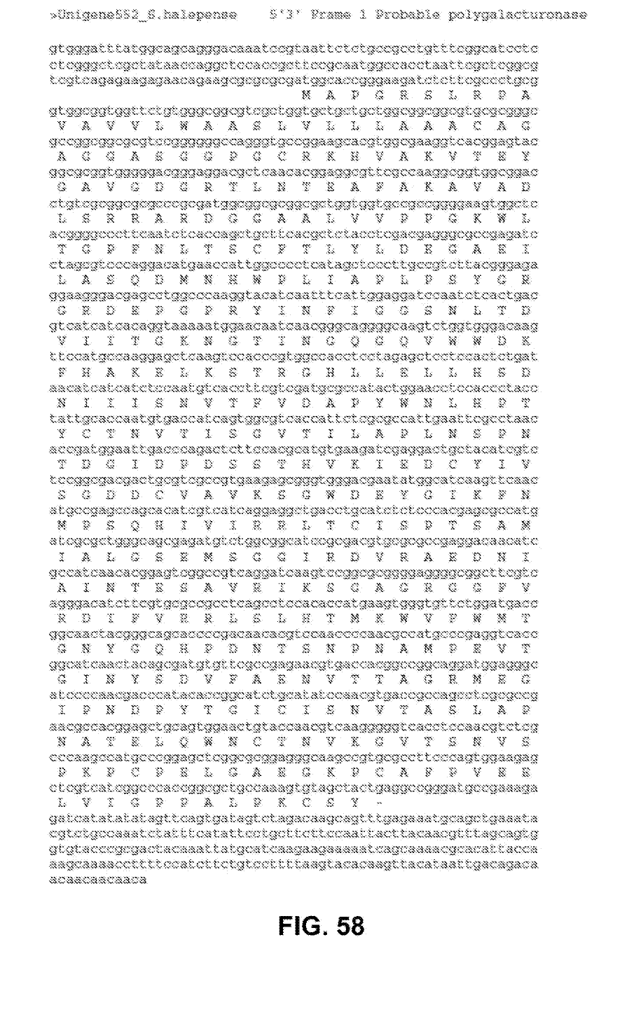

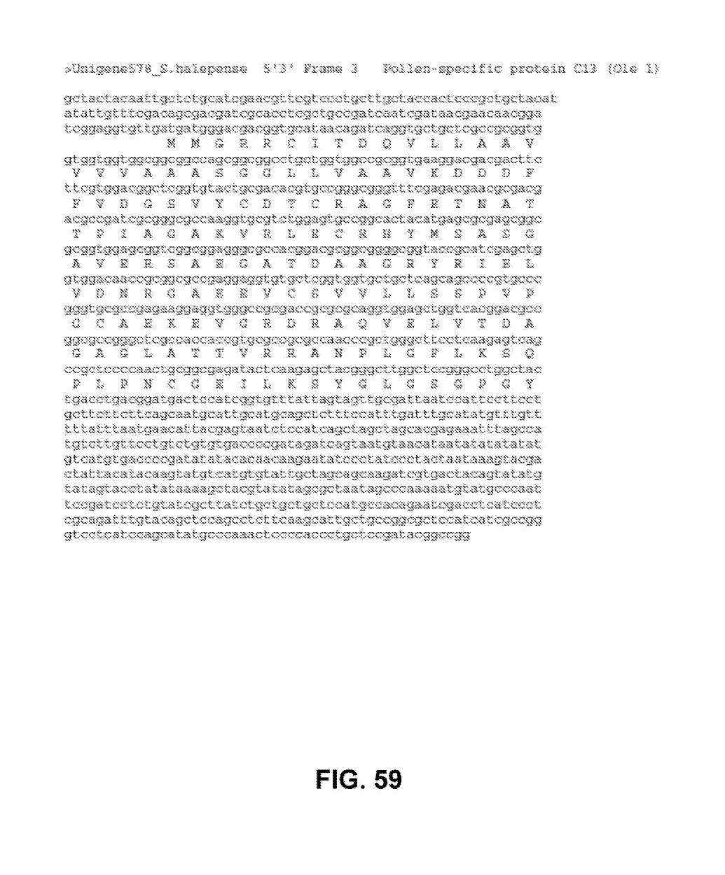

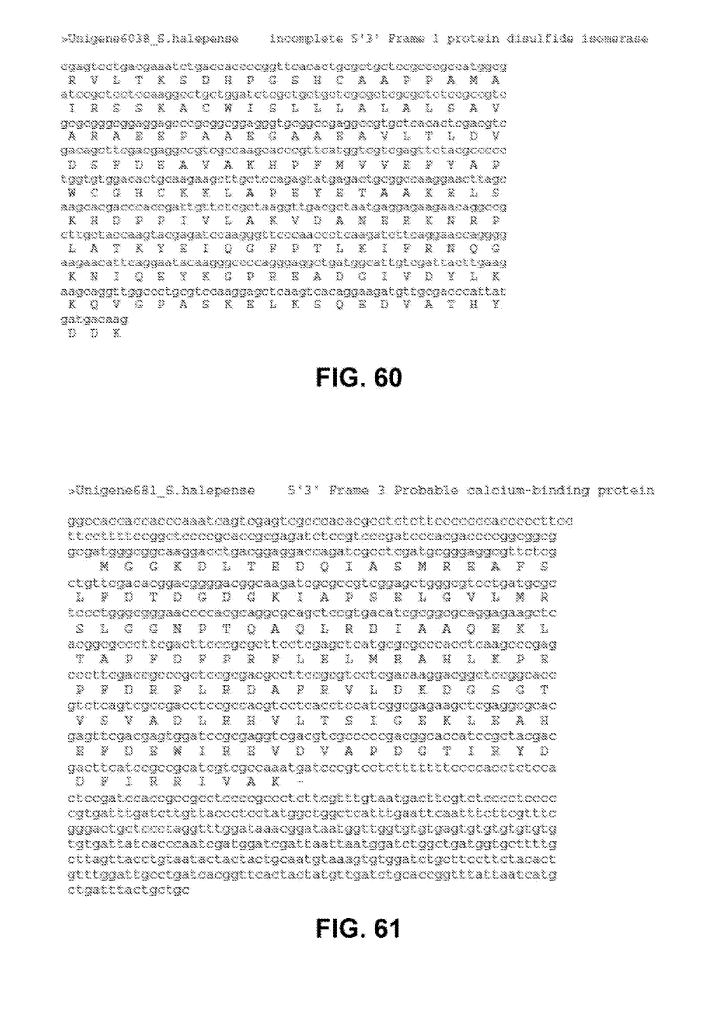

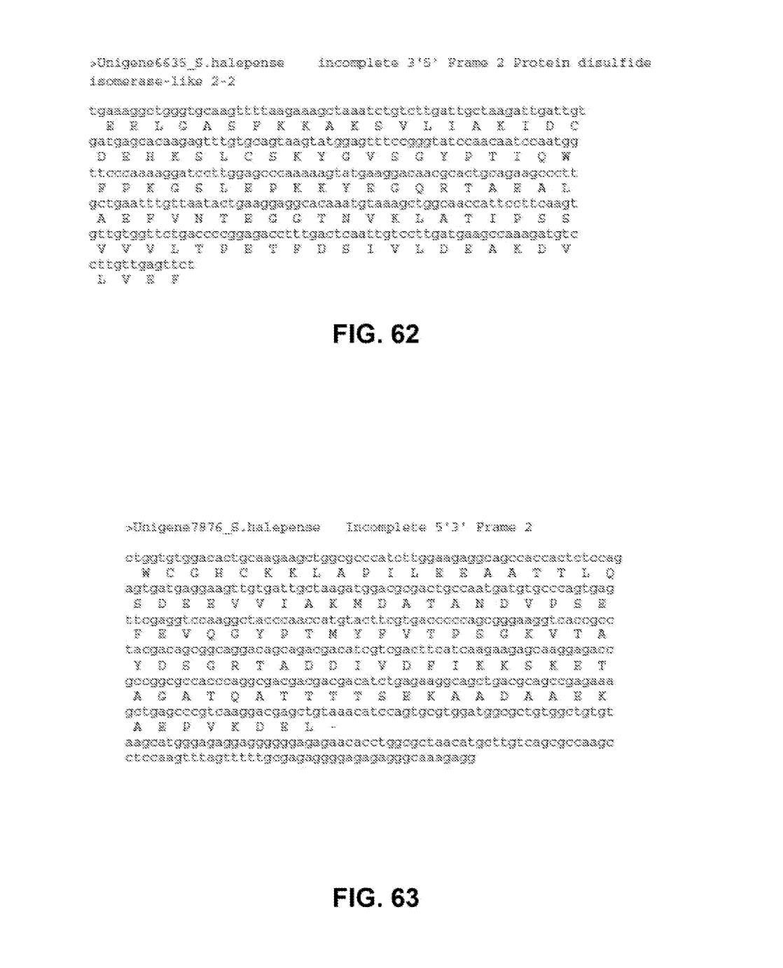

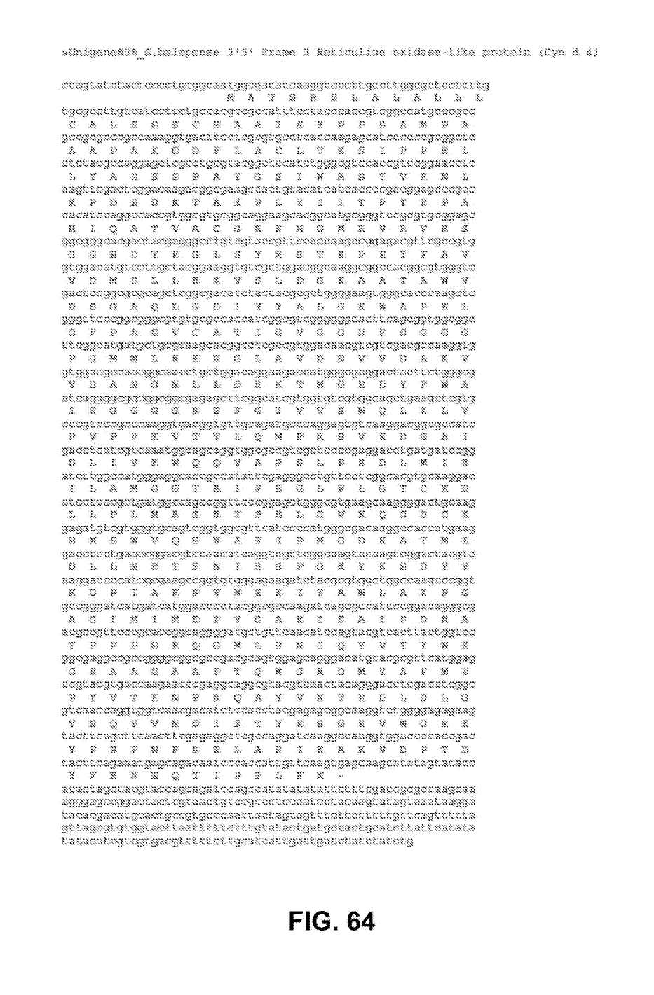

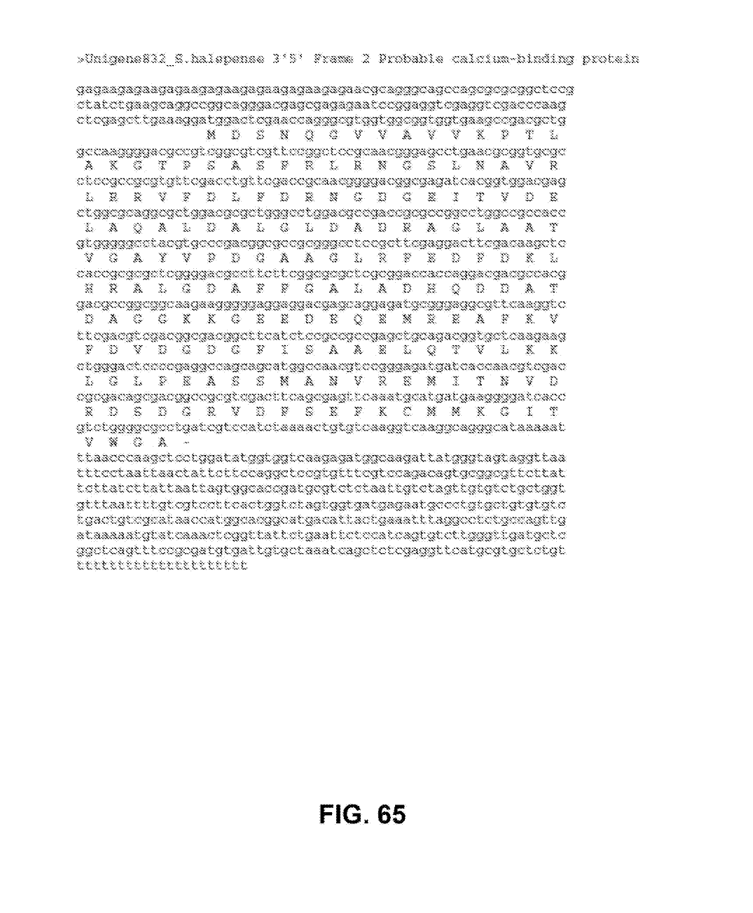

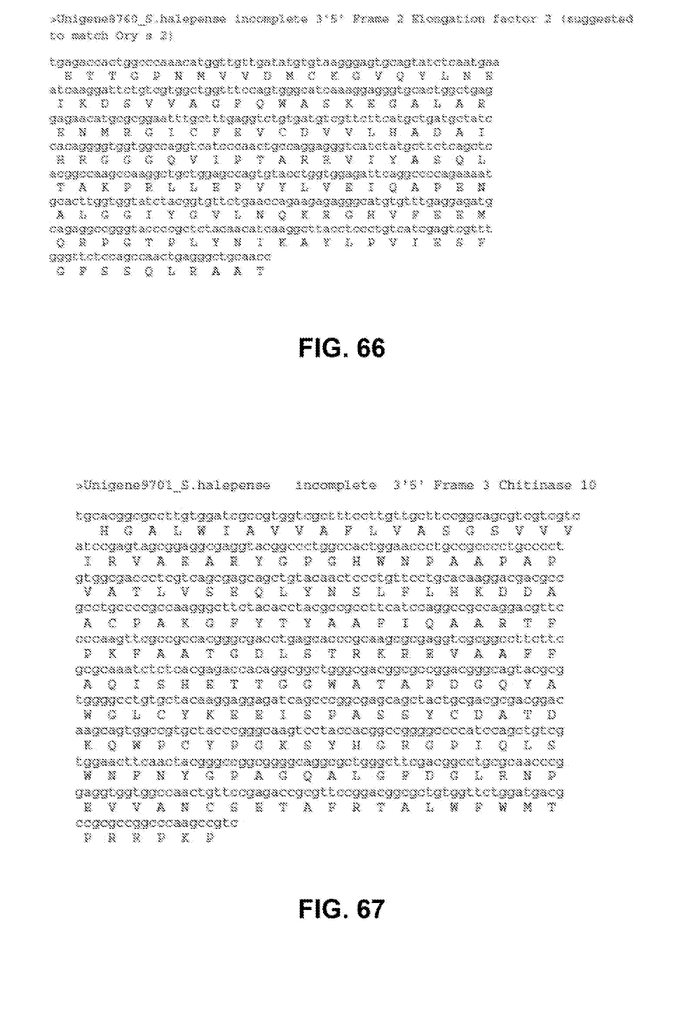

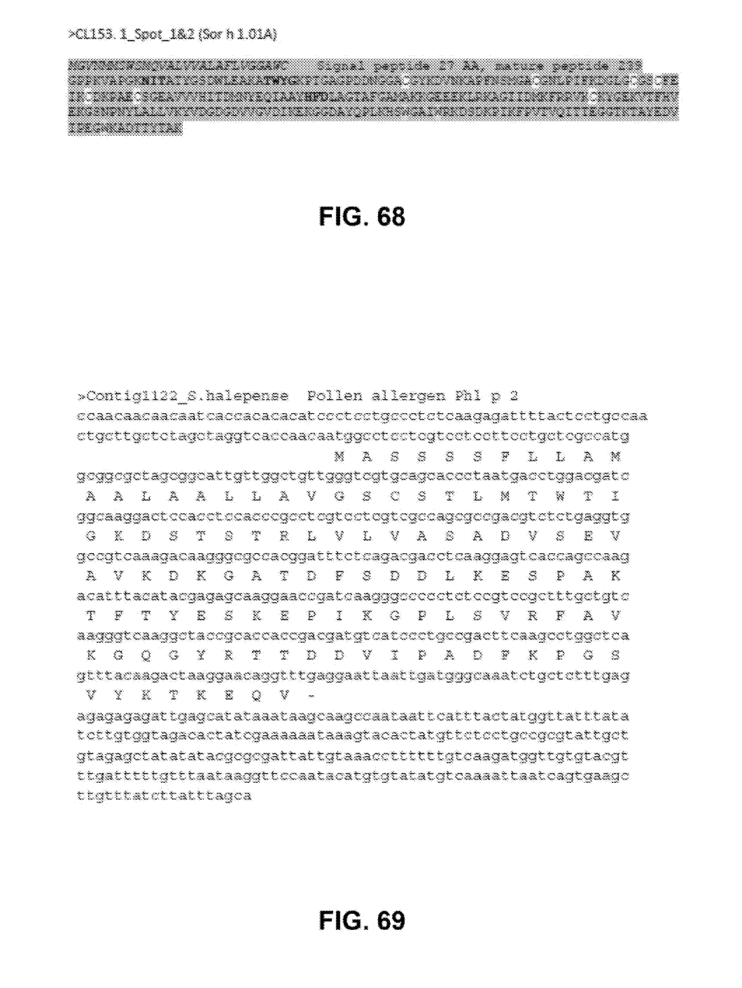

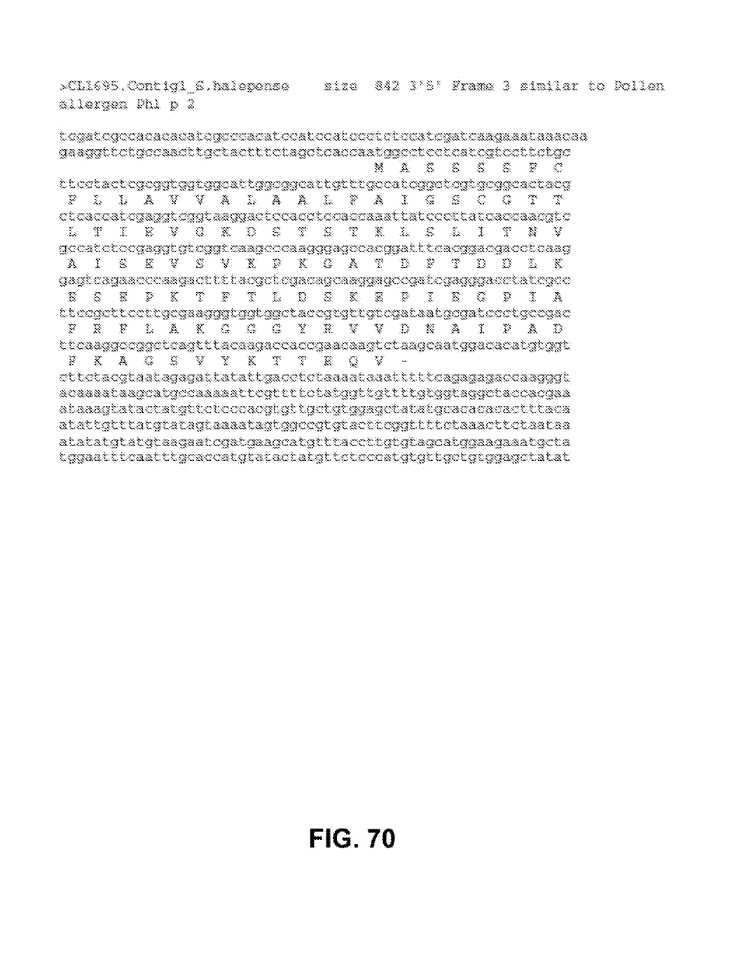

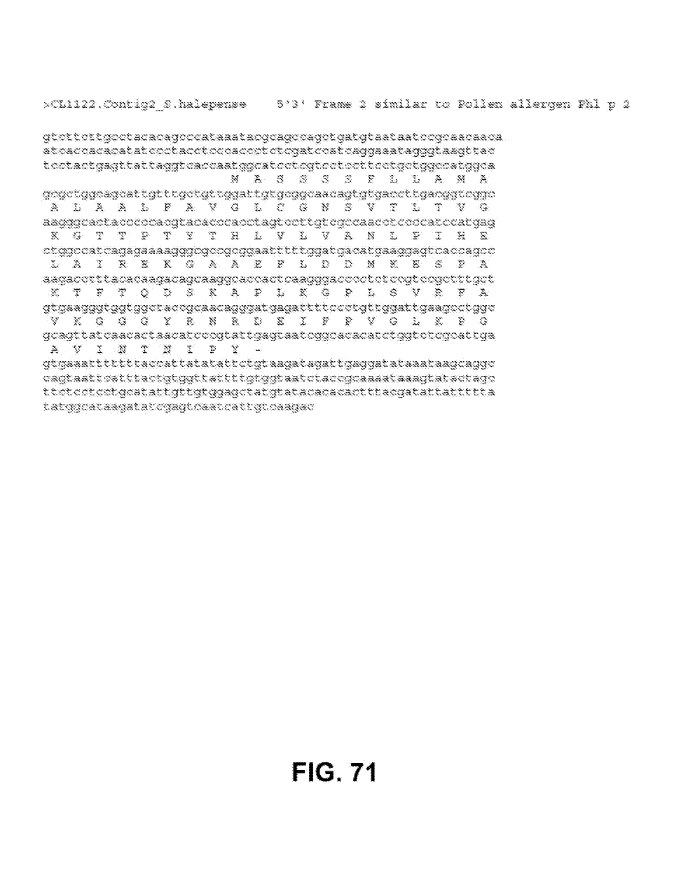

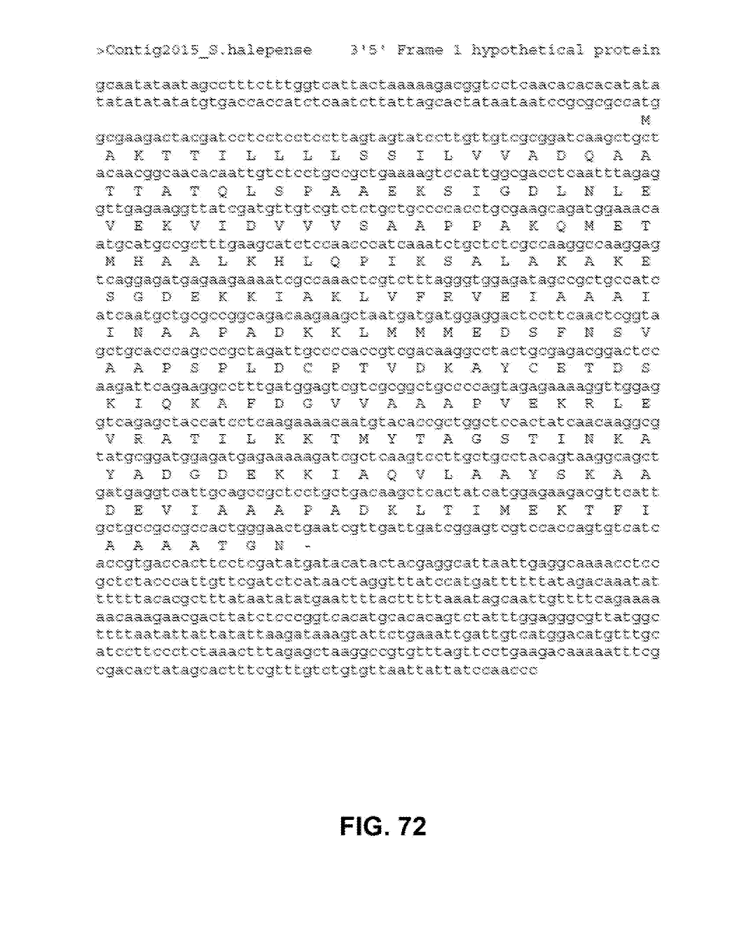

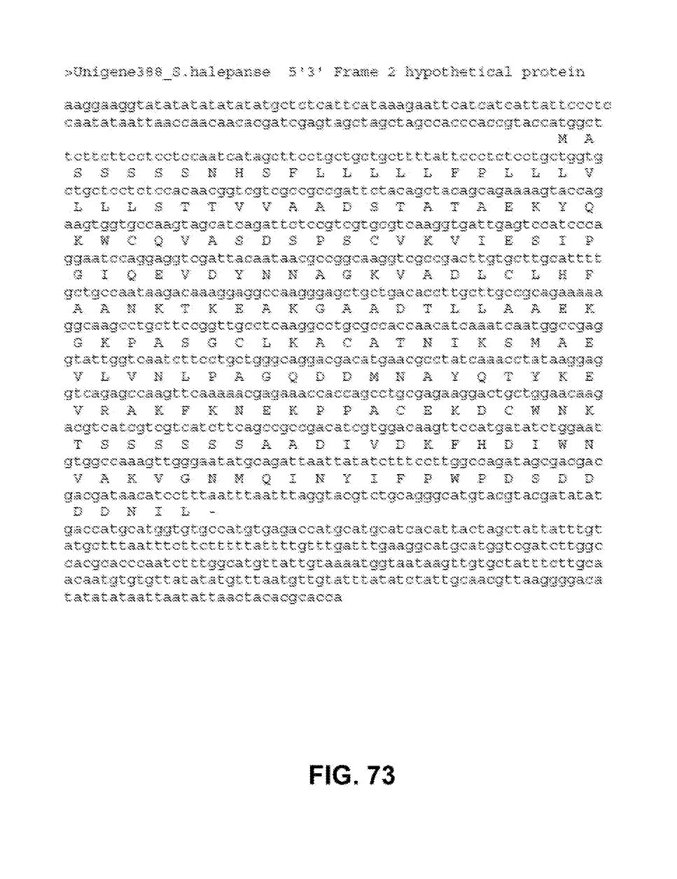

SEQ ID NO: 1=peptide sequence Sor h 1.02B of FIG. 28; sequence includes 24 amino acid signal peptide (total=266 amino acids)
SEQ ID NO: 2=peptide sequence Sor h 1.02B (mature peptide) of FIG. 28; sequence excludes 24 amino acid signal peptide (total=242 amino acids)
SEQ ID NO: 3=peptide sequence CL1737.1 (Sor h 13.01) of FIG. 29; sequence includes 23 amino acid signal peptide (total=422 amino acids)
SEQ ID NO: 4=peptide sequence CL1737.1 (Sor h 13.01, mature peptide) of FIG. 29; sequence excludes 23 amino acid signal peptide (total=399 amino acids)
SEQ ID NO: 5=peptide sequence CL1737.2 (Sor h 13.02) of FIG. 30; sequence includes 22 amino acid signal peptide (total=410 amino acids)
SEQ ID NO: 6=peptide sequence CL1737.2 (Sor h 13.02, mature peptide) of FIG. 30; sequence excludes 22 amino acid signal peptide (total=388 amino acids)
SEQ ID NO: 7=peptide sequence Contig110 of FIG. 31
SEQ ID NO: 8=peptide sequence CL1152 of FIG. 32
SEQ ID NO: 9=peptide sequence CL1715 of FIG. 33
SEQ ID NO: 10=peptide sequence CL1444 of FIG. 34
SEQ ID NO: 11=peptide sequence CL1754 of FIG. 35
SEQ ID NO: 12=peptide sequence CL200 of FIG. 36
SEQ ID NO: 13=peptide sequence CL2015.2 of FIG. 37
SEQ ID NO: 14=peptide sequence CL2052 of FIG. 38
SEQ ID NO: 15=peptide sequence CL248 of FIG. 39
SEQ ID NO: 16=peptide sequence CL70 of FIG. 40
SEQ ID NO: 17 w peptide sequence CL830 of FIG. 41
SEQ ID NO: 18=peptide sequence CL962 of FIG. 42
SEQ ID NO: 19=peptide sequence CL986 of FIG. 43
SEQ ID NO: 20=peptide sequence UG1043 of FIG. 44
SEQ ID NO: 21=peptide sequence UG11756 of FIG. 45
SEQ ID NO: 22=peptide sequence UG1334 of FIG. 46
SEQ ID NO: 23=peptide sequence UG01403 of FIG. 47
SEQ ID NO: 24=peptide sequence UG2745 of FIG. 48
SEQ ID NO: 25=peptide sequence UG308 of FIG. 49
SEQ ID NO: 26=peptide sequence UG332 of FIG. 50
SEQ ID NO: 27=peptide sequence UG335 of FIG. 51
SEQ ID NO: 28=peptide sequence UG342 of FIG. 52
SEQ ID NO: 29=peptide sequence UG397 of FIG. 53
SEQ ID NO: 30=peptide sequence UG451 of FIG. 54
SEQ ID NO: 31=peptide sequence UG540 of FIG. 55
SEQ ID NO: 32=peptide sequence UG5446 of FIG. 56
SEQ ID NO: 33=peptide sequence UG551 of FIG. 57
SEQ ID NO: 34=peptide sequence UG552 of FIG. 58
SEQ ID NO: 35=peptide sequence UG578 of FIG. 59
SEQ ID NO: 36=peptide sequence UG6038 of FIG. 60
SEQ ID NO: 37=peptide sequence UG681 of FIG. 61
SEQ ID NO: 38=peptide sequence UG6635 of FIG. 62
SEQ ID NO: 39=peptide sequence UG7876 of FIG. 63
SEQ ID NO: 40=peptide sequence UG808 of FIG. 64
SEQ ID NO: 41=peptide sequence UG832 of FIG. 65
SEQ ID NO: 42=peptide sequence UG8760 of FIG. 66
SEQ ID NO: 43=peptide sequence UG9701 of FIG. 67
SEQ ID NO: 44=peptide sequence CL153.1 (Sor h 1.01A) of FIG. 68; sequence includes 27 amino acid signal peptide (total=266 amino acids)
SEQ ID NO: 45=peptide sequence CL1122.1 (Sor h 2.01) of FIG. 69
SEQ ID NO: 46=peptide sequence CL1695 (Sor h 2.02) of FIG. 70
SEQ ID NO: 47=peptide sequence CL1122.2 (Sor h 2.03) of FIG. 71
SEQ ID NO; 48=peptide sequence CL2015.1 (Sor h 23) of FIG. 72
SEQ ID NO: 49=peptide sequence CL1388/UG388 (Spot 6) of FIG. 73
SEQ ID NO: 50=nucleic acid sequence of Sor h 1.02 (UG492-UG493) transcript of FIG. 28; the ATG start and Stop codons are highlighted.
SEQ ID NO: 51=nucleic acid sequence of Sor b 13.01 (CL1737.1) transcript of FIG. 29; the coding sequence from the ATG start codon to the TGA stop codon is underlined.
SEQ ID NO: 52=nucleic acid sequence of Sor h 13.02 (CL1737.2) transcript of FIG. 30; the coding sequence from the ATG start codon to the TGA stop codon is underlined.
SEQ ID NO: 53=nucleic acid coding sequence Contig110 of FIG. 31
SEQ ID NO: 54=nucleic acid coding sequence CL1152 of FIG. 32
SEQ ID NO: 55=nucleic acid coding sequence CL1715 of FIG. 33
SEQ ID NO: 56=nucleic acid coding sequence CL1444 of FIG. 34
SEQ ID NO: 57=nucleic acid coding sequence CL1754 of FIG. 35
SEQ ID NO: 58=nucleic acid coding sequence CL200 of FIG. 36
SEQ ID NO: 59=nucleic acid coding sequence CL2015.2 of FIG. 37
SEQ ID NO: 60=nucleic acid coding sequence CL2052 of FIG. 38
SEQ ID NO: 61=nucleic acid coding sequence CL248 of FIG. 39
SEQ ID NO: 62=nucleic acid coding sequence CL70 of FIG. 40
SEQ ID NO: 63=nucleic acid coding sequence CL830 of FIG. 41
SEQ ID NO: 64=nucleic acid coding sequence CL962 of FIG. 42
SEQ ID NO: 65=nucleic acid coding sequence CL986 of FIG. 43
SEQ ID NO: 66=nucleic acid coding sequence UG1043 of FIG. 44
SEQ ID NO: 67=nucleic acid coding sequence UG11756 of FIG. 45
SEQ ID NO: 68=nucleic acid coding sequence UG1334 of FIG. 46
SEQ ID NO: 69=nucleic acid coding sequence UG1403 of FIG. 47
SEQ ID NO: 70=nucleic acid coding sequence UG2745 of FIG. 48
SEQ ID NO: 71=nucleic acid coding sequence UG308 of FIG. 49
SEQ ID NO: 72=nucleic acid coding sequence UG332 of FIG. 50
SEQ ID NO: 73=nucleic acid coding sequence UG335 of FIG. 51
SEQ ID NO: 74=nucleic acid coding sequence UG342 of FIG. 52
SEQ ID NO: 75=nucleic acid coding sequence UG397 of FIG. 53
SEQ ID NO: 76=nucleic acid coding sequence UG451 of FIG. 54
SEQ ID NO: 77=nucleic acid coding sequence UG540 of FIG. 55
SEQ ID NO: 78=nucleic acid coding sequence UG5446 of FIG. 56
SEQ ID NO: 79=nucleic acid coding sequence UG551 of FIG. 57
SEQ ID NO: 80=nucleic acid coding sequence UG552 of FIG. 58
SEQ ID NO: 81=nucleic acid coding sequence UG578 of FIG. 59
SEQ ID NO: 82=nucleic acid coding sequence UG6038 of FIG. 60
SEQ ID NO: 83=nucleic acid coding sequence UG681 of FIG. 61
SEQ ID NO: 84=nucleic acid coding sequence UG6635 of FIG. 62
SEQ ID NO: 85=nucleic acid coding sequence UG7876 of FIG. 63
SEQ ID NO: 86=nucleic acid coding sequence UG808 of FIG. 64
SEQ ID NO: 87=nucleic acid coding sequence UG832 of FIG. 65
SEQ ID NO: 88=nucleic acid coding sequence UG8760 of FIG. 66
SEQ ID NO: 89=nucleic acid coding sequence UG9701 of FIG. 67
SEQ ID NO: 90=nucleic acid coding sequence CL1122.1 (Sor h 2.01) of FIG. 69
SEQ ID NO: 91=nucleic acid coding sequence CL1695 (Sor h 2.02) of FIG. 70
SEQ ID NO: 92=nucleic acid coding sequence CL1122.2 (Sor h 2.03) of FIG. 71
SEQ ID NO: 93=nucleic acid coding sequence 2015.1 (Sor h 23) of FIG. 72
SEQ ID NO: 94=nucleic acid coding sequence CL1388/UG388 (Spot 6) of FIG. 73

### DETAILED DESCRIPTION

The present invention is at least partly predicated on the first detailed bioinformatic and clinical characterisation of the pollen from the subtropical grass Sorghum halepense (Johnson grass; Panicoideae), a wind pollinated perennial grass found worldwide and considered a major weed and significant source of allergenicity in the subtropics including parts of Australia, Africa, Asia and the Americas.

Integrating modern transcriptomic sequencing technology with advanced proteomic and serological analysis has allowed a comprehensive analysis of mature Johnson grass pollen allergen diversity. Furthermore, serum IgE reactivities with pollen and purified allergens were assessed in 64 patients with grass pollen allergy from a subtropical region. IgE of patients with allergic sensitivity to JGP reacted with two dominant allergenic components; Sor h 1 and the newly identified Sor h 13. Serum IgE with purified Sor h 1 was observed in 40 of 41 patients with IgE reactivity to JGP (97.5%) as well as nine grass pollen-allergic patients without IgE to JGP (76% overall). IgE reactivity with JGP and Sor h 1 were highly correlated (r=0.9686, p<0.0001). IgE reactivity with Sot h 13 was observed in 28 of the grass pollen-allergic donors (43.7% overall). Five additional JGP components showed IgE reactivity. cDNA transcripts and peptides of JGP belonging to allergen families 2, 4, 11 and 12 were identified. Group 5 and 6 allergen families were not clearly apparent, whereas homologues of Bermuda grass allergen (groups 15, 22 and 23) were present. Knowledge of the allergenic components of subtropical grass pollens, such as those from Johnson grass, should facilitate increased understanding of the contribution to the disease burden of allergic rhinitis in subtropical regions of the world.

The present invention also includes the identification of previously unknown and/or novel grass pollen allergens from Johnson grass (Sorghum halepense).

In one aspect, the invention provides a method for determining sensitivity to a Johnson grass (Sorghum halepense) pollen allergen, or an allergen immunologically cross-reactive with a Johnson grass pollen allergen, in a subject (e.g., a human), including the step of determining a presence or absence of an allergen-specific immune response in said subject, wherein the presence of said immune response indicates sensitivity to the Johnson grass pollen allergen or said immunologically cross-reactive allergen.

Suitably, sensitivity to the Johnson grass pollen allergen is associated with an allergic condition.

Preferably, the allergic condition is allergic rhinitis, allergic asthma or allergic dermatitis.

As used herein, “sensitive” and “sensitivity”, in the context of allergy, mean that an individual is susceptible to, or has an increased likelihood or probability of following contact with that particular allergen, inducing an allergen-specific immune response. This includes situations where the individual is not yet exhibiting clinical symptoms of sensitivity or allergy as well as where the individual is displaying symptoms of sensitivity or allergy.

By “immune response” is meant the response of a subject's immune system comprising recognizing and responding to an immunogen, such as an allergen, which may neutralize and/or remove said immunogen from the subject. Immunogens may be on the surface of cells, viruses, fungi, or bacteria or may be nonliving substances such as toxins, chemicals, drugs, and foreign particles. An allergen is a type of immunogen that produces an abnormal or aberrant immune response in which the subject's immune system recognises and responds to a perceived harmful immunogen (i.e., the allergen) that would otherwise be largely harmless to the body.

A subject's immune response to an allergen may comprise the production of allergen-specific antibodies, such as IgE, by cells of the subject's immune system. As would be acknowledged by those skilled in the art, allergy or allergic conditions at least partly involve circulating IgE that binds to high-affinity IgE receptors on immune effector cells (e.g. mast cells) located throughout the body triggering mast cell degranulation and an immediate allergic response. Such responses may comprise the release of histamine, leukotrienes, cytokines or other immunologically relevant mediators from allergy effector cells, such as basophils, mast cells or cosinophils. The allergic response in human beings may also be, at least partly, mediated by T lymphocytes, which may proliferate and/or secrete cytokines, such as IL-4, IL-5, and IL-13, in response to activation by allergen-derived peptides.

Allergic conditions commonly include signs and symptoms that can be: (i) cutaneous (e.g. urticaria); (ii) respiratory (e.g. acute bronchospasm, rhinoconjunctivitis); (iii) cardiovascular (e.g. tachycardia, hypotension); (iv) gastrointestinal (e.g. vomiting, diarrhoea); and/or (v) systemic (e.g. anaphylactic shock) in nature.

It would be understood by those skilled in the art that the Johnson grass pollen allergens disclosed herein may be used to detect antibodies or immune cell responses directed against said allergens in vitro or in vivo. Such in vitro testing may involve obtaining a biological sample, such as blood or serum, from the subject. The detection of an antibody or elevated levels of an antibody in the biological sample from a subject may be indicative of sensitization or allergy to a Johnson grass pollen allergen in said subject.

“Elevated levels of antibody” represent a higher than normal level of an antibody or antibodies specific to a particular allergen in their biological sample, when compared to a sample obtained from a subject not exposed to the allergen or to the general population. For example, a subject demonstrating elevated levels of antibody to a specific pollen allergen may be considered to be sensitive to or have a sensitivity to, or may be considered to be allergic or have an allergy to, that particular pollen allergen.

Suitable techniques at measuring the level of antibody specific to a particular allergen are well known in the art. Such techniques typically involve immunoassays, such as western blots, enzyme-linked immunosorbent assays (ELISAs), fluorescent enzyme immunoassays (FEIAs), and radioallergosorbent assays (RASTs). At present, most commercial laboratories use one of three autoanalyzer systems to measure allergen-specific antibody: (i) ImmunoCAP (Thermofisher, formerly Phadia AB, Uppsala, Sweden); (ii) Immulite (Siemens AG, Berlin, Germany); or (iii) HYTEC-288 (Hycor/Agilent, Garden Grove, Calif.). The tests can be used to evaluate sensitivity to various allergens, including common inhalants such as pollens.

It would be further appreciated, that combinations of specific antibody tests, and in particular specific IgE tests, for allergen components of Johnson grass pollen have potential use in characterising the risk profiles of disease progression and disease severity, establishing a primary source of allergic sensitisation, selecting patients for allergen immunotherapy treatment and guiding the choice of appropriate allergens for immunotherapy of a given patient.

Where the concentration of the antibodies is determined, quantitation of the antibody response may be repeated over time. This may include monitoring the efficacy of allergen-specific immunotherapy or desensitisation therapy administered to a subject. Additionally, this may include monitoring disease progression and/or severity.

Suitably, determining a presence or absence of an allergen-specific immune response involves detection of an allergen-specific antibody or antibodies

Preferably, the allergen-specific antibody is of the IgM, IgE, IgG or IgA class.

More preferably, the allergen-specific antibody is an IgE antibody.

The Johnson grass pollen allergens of the current invention may also be used for cell-specific tests, including but not limited to a T-cell proliferation test and a basophil mediator release test. The allergens may be administered to various cell types, including allergy effector cells, to invoke measurable responses, such as histamine and/or cytokine release. In another type of assay, the proliferation (e.g., ³H Thymidine uptake), apoptosis (e.g., Annexin V positivity) or death (e.g., propidium iodide positivity) of cells, such as T cells or peripheral blood mononuclear cells, may be determined.

The Johnson grass pollen allergens may also be used for in vivo diagnostic purposes, such as in vivo provocation testing. Such tests may comprise skin testing (e.g., skin prick testing), nasal provocation testing, allergen aerosol chamber challenge, bronchial provocation testing or food challenge testing.

By “immunologically cross-reactive” in the context of allergens is meant the ability of an individual allergen-specific antibody and/or other elements of the immune response to recognise and react with more than one particular allergen. Immunological cross-reactivity arises, as would be appreciated by a skilled artisan, because the immunologically cross-reactive allergen has an epitope or antigenic determinant in common with or has an epitope or antigenic determinant which is structurally similar to the sensitizing allergen. Since Johnson grass pollen allergens according to the invention may contain one or more epitopes or antigenic determinants (or similar epitopes or antigenic determinants) of unrelated allergens, they may also be used for diagnostic screening/monitoring tests and/or preventative/therapeutic immunotherapy (as described herein) for these unrelated allergens.

In particular embodiments, the Johnson grass pollen allergen comprises an isolated allergenic protein comprising, consisting of or consisting essentially of an amino acid sequence set forth in SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48 or SEQ ID NO: 49.

In this context, by “consisting essentially of” means that the isolated protein comprises the amino acid sequence of any one of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48 or SEQ ID NO: 49 together with 1, 2, 3, 4 or 5 additional amino acids at the N- and/or C-terminus.

In another aspect, the invention provides an isolated protein which comprises, consists essentially oft or consists of an amino acid sequence set forth in SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42 or SEQ ID NO: 43.

In particular embodiments, the isolated protein comprises an amino acid sequence set forth in SEQ ID NO: 1, SEQ ID NO: 2, SEQ ID NO: 3, SEQ ID NO: 4, SEQ ID NO: 5 or SEQ ID NO: 6.

For the purposes of this invention, by “Isolated” is meant material that has been removed from its natural state or otherwise been subjected to human manipulation. Isolated material may be substantially or essentially free from components that normally accompany it in its natural state, or may be manipulated so as to be in an artificial state together with components that normally accompany it in its natural state. Isolated material includes material in native and recombinant form. The term “isolated” also encompasses terms such as “enriched”, “purified” and/or “synthetic”. Synthetic includes recombinant synthetic and chemical synthetic.

By “protein” is meant an amino acid polymer. The amino acids may be natural or non-natural amino acids, D- or L-amino acids, as are well understood in the art.

A “peptide” is a protein having no more than sixty (60) amino acids.

A polypeptide is a protein having more than sixty (60) amino acids.

In further embodiments, the isolated allergenic protein, comprising, consisting of or consisting essentially of an amino acid sequence set forth in SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42 or SEQ ID NO: 43 is a recombinant protein.

This aspect also includes fragments, variants and derivatives of said isolated protein.

In this regard, a protein “fragment” includes an amino acid sequence that constitutes less than 100%, but at least 20%, 30%, 40%, 50%, 60%, 70%, 80% or 90%, 92%, 94%, 96%, 98%, or 99% of said isolated allergenic protein.

In particular aspects, a protein fragment may comprise, for example, at least 10, 15, 20, 25, 30 35, 40, 45, 50, 55, 60, 65, 70, 75, 80, 85, 90, 95, 100, 125, 150, 175, 200, 225, 250, 275, 300, 325, 350, 375 and 400 contiguous amino acids of said allergenic protein.

It will be appreciated that a peptide may be a protein fragment, for example comprising at least 6, 10, 12 preferably at least 15, 20, 25, 30, 35, 40, 45, and more preferably at least 50 contiguous amino acids.

Peptide fragments may be obtained through the application of standard recombinant nucleic acid techniques or synthesized using conventional liquid or solid phase synthesis techniques. For example, reference may be made to solution synthesis or solid phase synthesis as described, for example, in Chapter 18 of CURRENT PROTOCOLS IN PROTEIN SCIENCE, Coligan et al. Eds (John Wiley & Sons, 1995-2000). Alternatively, peptides can be produced by digestion of an allergenic protein of the invention with proteases such as endoLys-C, endoArg-C, endoGlu-C and staphylococcus V8-protease. The digested fragments can be purified by, for example, high performance liquid chromatographic (HPLC) techniques as are well known in the art.

It will also be appreciated that larger peptides and isolated allergenic proteins comprising a plurality of the same or different fragments are contemplated.

The invention also provides variants of the allergenic proteins.

As used herein, a protein “variant” shares a definable nucleotide or amino acid sequence relationship with an isolated protein disclosed herein. Preferably, protein variants share at least 70% or 75%, preferably at least 80% or 85% or more preferably at least 90%, 91%, 92%, 93%, 94%, 95%, 96%, 97%, 98% or 99% sequence identity with the amino acid sequences of the invention.

As used herein “variant” proteins disclosed herein have one or more amino acids deleted or substituted by different amino acids. It is well understood in the art that some amino acids may be substituted or deleted without changing the activity of the allergenic protein (conservative substitutions).

The term “variant” also includes isolated proteins disclosed herein produced from, or comprising amino acid sequences of, allelic variants.

Terms used generally herein to describe sequence relationships between respective proteins and nucleic acids include “comparison window”, “sequence identity” “percentage of sequence identity” and “substantial identity”. Because respective nucleic acids/proteins may each comprise (1) only one or more portions of a complete nucleic acid/protein sequence that are shared by the nucleic acids/proteins, and (2) one or more portions which are divergent between the nucleic acids/proteins, sequence comparisons are typically performed by comparing sequences over a “comparison window” to identify and compare local regions of sequence similarity. A “comparison window” refers to a conceptual segment of typically 6, 9 or 12 contiguous residues that is compared to a reference sequence. The comparison window may comprise additions or deletions (i.e., gaps) of about 20% or less as compared to the reference sequence for optimal alignment of the respective sequences. Optimal alignment of sequences for aligning a comparison window may be conducted by computerised implementations of algorithms (Geneworks program by Intelligenetics; GAP, BESTFIT, FASTA, and TFASTA in the Wisconsin Genetics Software Package Release 7.0, Genetics Computer Group, 575 Science Drive Madison, Wis., USA, incorporated herein by reference) or by inspection and the best alignment (i.e. resulting in the highest percentage homology over the comparison window) generated by any of the various methods selected. Reference also may be made to the BLAST family of programs as for example disclosed by Altschul et al., 1997, Nucl. Acids Res. 25 3389, which is incorporated herein by reference. A detailed discussion of sequence analysis can be found in Unit 19.3 of CURRENT PROTOCOLS IN MOLECULAR BIOLOGY Eds. Ausubel et al. (John Wiley & Sons Inc NY, 1995-1999).

The term “sequence identity” is used herein in its broadest sense to include the number of exact nucleotide or amino acid matches having regard to an appropriate alignment using a standard algorithm, having regard to the extent that sequences are identical over a window of comparison. Thus, a “percentage of sequence identity” is calculated by comparing two optimally aligned sequences over the window of comparison, determining the number of positions at which the identical nucleic acid 20 base (e.g., A, T, C, G, I) occurs in both sequences to yield the number of matched positions, dividing the number of matched positions by the total number of positions in the window of comparison (i.e., the window size), and multiplying the result by 100 to yield the percentage of sequence identity. For example, “sequence identity” may be understood to mean the “match percentage” calculated by the DNASIS computer program (Version 2.5 for windows; available from Hitachi Software engineering Co., Ltd., South San Francisco, Calif., USA). Preferably, sequence identity is measured over the entire amino acid sequence of the Johnson grass allergen.

Derivatives of the allergenic proteins are also provided.

As used herein, “derivative” proteins have been altered, for example by conjugation or complexing with other chemical moieties, by post-translational modification (e.g., phosphorylation, acetylation and the like), modification of glycosylation (e.g., adding, removing or altering glycosylation) and/or inclusion of additional amino acid sequences as would be understood in the art.

Additional amino acid sequences may include fusion partner amino acid sequences which create a fusion protein. By way of example, fusion partner amino acid sequences may assist in detection and/or purification of the isolated fusion protein. Non-limiting examples include metal-binding (e.g., polyhistidine) fusion partners, maltose binding protein (MBP), Protein A, glutathione S-transferase (GST), fluorescent protein sequences (e.g., GFP), epitope tags such as myc, FLAG and haemagglutinin tags.

Other derivatives contemplated by the invention include, but are not limited to, modification to side chains, incorporation of unnatural amino acids and/or their derivatives during peptide, or protein synthesis and the use of crosslinkers and other methods which impose conformational constraints on the allergenic proteins, fragments and variants of the invention.

Specifically, allergen derivatives may be produced with the aim of reducing their allergenicity without affecting their immunogenicity. Such allergen derivatives may therefore achieve similar or improved immunotherapy or desensitisation results with fewer treatments or a shorter course of treatments. Allergen derivatives for use in immunotherapy or desensitisation are well known to the skilled artisan. Non-limiting examples include allergens that have been polymerised, formaldehyde treated or specifically mutated.

In a further aspect, the invention provides an antibody or antibody fragment which binds and/or is raised against an isolated protein comprising an amino acid sequence according to SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42 or SEQ ID NO: 43. Suitably, said antibody or antibody fragment specifically bind the isolated protein comprising said amino acid sequence.

Antibodies of the invention may be polyclonal or monoclonal, native or recombinant. Well-known protocols applicable to antibody production, purification and use may be found, for example, in Chapter 2 of Coligan et at, CURRENT PROTOCOLS IN IMMUNOLOGY (John Wiley & Sons NY, 1991-1994) and Harlow, E. & Lane, D. Antibodies: A Laboratory Manual, Cold Spring Harbor, Cold Spring Harbor Laboratory, 1988, which are both herein incorporated by reference.

Generally, antibodies of the invention bind to or conjugate with an isolated protein, fragment, variant, or derivative disclosed herein. For example, the antibodies may be polyclonal antibodies. Such antibodies may be prepared for example by injecting an isolated protein, fragment, variant or derivative of the invention into a production species, which may include mice, rats or rabbits, to obtain polyclonal antisera. Methods of producing polyclonal antibodies are well known to those skilled in the art. Exemplary protocols which may be used are described for example in Coligan et al., CURRENT PROTOCOLS IN IMMUNOLOGY, supra, and in Harlow & Lane, 1988, supra.

Monoclonal antibodies may be produced using the standard method as for example, described in an article by Köhler & Milstein, 1975, Nature 256, 495, which is herein incorporated by reference, or by more recent modifications thereof as for example, described in Coligan et al., CURRENT PROTOCOLS IN IMMUNOLOGY, supra by immortalizing spleen or other antibody producing cells derived from a production species which has been inoculated with one or more of the isolated proteins, fragments, variants or derivatives of the invention.

The invention also includes within its scope antibody fragments, such as Fc, Fab or F(ab)2 fragments of the polyclonal or monoclonal antibodies referred to above. Alternatively, the antibodies may comprise single chain Fv antibodies (scFvs) against the peptides of the invention. Such scFvs may be prepared, for example, in accordance with the methods described respectively in U.S. Pat. No. 5,091,513, European Patent No 239,400 or the article by Winter & Milstein, 1991, Nature 349:293, which are incorporated herein by reference. The invention is also contemplated to include multivalent recombinant antibody fragments, so-called diabodies, triabodies and/or tetrabodies, comprising a plurality of scFvs. By way of example, such antibodies may be prepared in accordance with the methods described in Holliger et al., 1993 Proc Natl Acad Sci USA 90:6444-6448; or in Kipriyanov, 2009 Methods Mol Biol 562:177-93 and herein incorporated by reference in their entirety.

Antibodies and antibody fragments of the invention may be particularly suitable for affinity chromatography purification of the allergenic proteins described herein. For example reference may be made to affinity chromatographic procedures described in Chapter 9.5 of Coligan et al., CURRENT PROTOCOLS IN IMMUNOLOGY, supra.

For diagnostic compositions and methods, the antibody or antibody fragment may be labelled. Non-limiting examples of labels include fluorescent labels (e.g FITC, Rhodamine, Texas Red and Coumarin, although without limitation thereto), enzyme labels (e.g. horseradish peroxidase or alkaline phosphatase, although without limitation thereto), radionuclides and/or digoxigenin, although without limitation thereto.

In particular embodiments, the antibody or antibody fragment is a recombinant antibody or antibody fragment.

It will be appreciated that an allergen or allergenic protein may bind with one or more allergen-specific antibodies to form an antibody-allergen complex. Binding typically takes place if an epitope or antigenic determinant of the allergen and can “fit into” or otherwise interact with one or more corresponding, specific antigen binding sites of the antibody. It will be well understood by a skilled artisan that most allergens will have multiple epitopes or antigenic determinants. Accordingly, a single antibody-allergen complex may contain more than one allergen-specific antibody.

In another aspect, the invention provides a method for measuring the level of or detecting or monitoring the presence of a Johnson grass pollen allergen, or an allergen immunologically cross-reactive with a Johnson grass pollen allergen, in a sample, including the step of contacting the sample with one or more reagents for a time and under conditions sufficient to detect said Johnson grass allergen or immunologically cross-reactive antigen.

In one embodiment, the one or more reagents are in the form of, or are present in, a diagnostic composition.

Suitably, the one or more reagents of this aspect of the invention include an antibody or fragment thereof.

Preferably, the antibody is polyclonal or monoclonal, native or recombinant.

Even more preferably, the antibody is a monoclonal antibody.

In particular embodiments, the one or more reagents comprises an antibody, or a fragment thereof, that binds and/or is raised against an isolated protein, or a fragment, variant or derivative thereof, comprising an amino acid sequence selected from the group consisting of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48 or SEQ ID NO: 49.

In one embodiment, the sample is an environmental sample. This particular embodiment of the invention may involve the acquisition of indoor samples, such as from homes, schools, commercial buildings and workplaces, and/or outdoor samples. For example, to detect and/or monitor pollen allergen levels in a household environment, a suitable sample may be collected dust. Other suitable samples may include, but are not limited to, soil, water, air, a foodstuff or a drink. Preferably, the environmental sample is air or water.

Suitably, the level of sensitivity is such that it will detect allergens which are present in the environment in concentrations at least which are just high enough to be clinically significant in that they are likely to elicit an immune response in a sensitive subject.

In another embodiment, the test sample is, or is derived from either a composition for immunotherapy or a diagnostic composition. In this regard, it is well appreciated that validated assays are required for the quality control of diagnostic and therapeutic compositions or products. These are applied at various stages of the manufacturing process to confirm batch-to-batch reproducibility and for final product clearance and release. Indeed, specifications and target values and stability data are typically submitted to regulatory bodies as part of the registration process. Amongst the most important requirement is the need for standardisation of the potency or levels of the active ingredient/s, and in particular the allergen/s, in the diagnostic or therapeutic composition or product to ensure batch-to-batch consistency (i.e., batch standardisation). Preferably, the method of this aspect is performed to batch standardize the pharmaceutical composition or the diagnostic composition.

Once collected the sample may be processed in a way, such as purifying, concentrating or solubilising, to make it more suitable for the subsequent allergen detection assay. Such assays, as would be readily understood by those skilled in the art, may include immunoassays, such as western blot and ELISA. It should be understood, however, that this invention is not limited by reference to the specific methods of detection or immunoassays disclosed.

Preferably, the antibodies of this aspect will be provided in molar excess to the levels of allergen that would be expected to be detected in a typical test sample.

In one embodiment, the sample comprises one or a plurality of other grass pollen-derived allergens in addition to said allergen. Such grass pollen-derived allergens may include one or more of those described herein.

Suitably, the method of this aspect is for determining a relative or absolute amount of the allergen in the sample.

Preferably, the levels of allergen detected in the test sample will be quantifiable.

In another aspect, the invention provides a method of preventing or treating sensitivity to a Johnson grass pollen allergen, or an allergen immunologically cross-reactive with a Johnson grass pollen allergen, in a subject, including the step of administering to said subject a composition comprising a therapeutically effective amount of a Johnson grass pollen allergen or an antibody thereto.

In one embodiment, the Johnson grass pollen allergen comprises an isolated protein, or a fragment, variant or derivative thereof, comprising an amino acid sequence selected from the group consisting of SEQ ID NOs: 1 to 49.

In a particular embodiment, the antibody, or a fragment thereof, binds and/or is raised against an isolated protein, or a fragment, variant or derivative thereof, comprising an amino acid sequence selected from the group consisting of SEQ ID NOs: 1 to 49.

Suitably, the composition to be administered comprises one or more pharmaceutically acceptable carriers, diluents or excipients as hereinafter described.

In one embodiment, the method of this aspect further comprises administering one or more additional allergens or one or more antibodies that bind and/or are raised against additional allergens. Such additional allergens may be one of those described herein. Preferably, the one ore more additional allergens include one or more grass pollen allergens from Bahia grass (Paspalum notatum), Bermuda grass (Cynodon dactylon) and/or Ryegrass (Lolium perenne).

In one embodiment, the therapeutically effective amount of the Johnson grass pollen allergen is administered subcutaneously.

In an alternative embodiment, the therapeutically effective amount of the Johnson grass pollen allergen is administered sublingually.

It will be appreciated by those skilled in the art that the methods of determining, preventing or treating sensitivity to a Johnson grass pollen allergen, or an allergen immunologically cross-reactive with a Johnson grass pollen allergen, described herein may be performed on any animal, inclusive of mammals such as domestic animals, livestock, performance animals and humans. Preferably, the subject is a human.

In yet another aspect, the invention provides a composition comprising an isolated protein comprising an amino acid sequence according to SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42; SEQ ID NO: 43, SEQ ID NO: 44, SEQ ID NO: 45; SEQ ID NO: 46; SEQ ID NO: 47; SEQ ID NO: 48 or SEQ ID NO: 49, a fragment, variant or derivative thereof, or an antibody which binds or is raised against said isolated protein.

In an embodiment, the isolated protein comprises an amino acid sequence according to any one of SEQ ID NOs: 1 to 43.

In an embodiment, the composition comprises one or more pharmaceutically acceptable carriers, diluents or excipients. Suitably, according to this embodiment the composition is suitable for treating or preventing sensitivity to a Johnson grass allergen.

As used herein, “treating” (or “treat” or “treatment”) refers to a therapeutic intervention that ameliorates a sign or symptom of allergen sensitivity after it has begun to develop. The term “ameliorating”, with reference to sensitivity, refers to any observable beneficial effect of the treatment. Treatment need not be absolute to be beneficial to the subject. The beneficial effect can be determined using any methods or standards known to the ordinarily skilled artisan.

As used herein, “preventing” (or “prevent” or “prevention”) refers to a course of action (such as administering a therapeutically effective amount of one or more Johnson grass pollen allergens or a biologically active fragment or variant thereof) initiated prior to the onset of a symptom, aspect, or characteristic of sensitivity so as to prevent or reduce the symptom, aspect, or characteristic. It is to be understood that such preventing need not be absolute to be beneficial to a subject. A “prophylactic” treatment is a treatment administered to a subject who does not exhibit signs of sensitivity or exhibits only early signs for the purpose of decreasing the risk of developing a symptom, aspect, or characteristic of sensitivity.

By “administration” is meant the introduction of a composition (e.g., a composition comprising one or more Johnson grass pollen allergens, or a biologically active fragment or variant thereof) into a subject by a chosen route.

The term “therapeutically effective amount” describes a quantity of a specified agent sufficient to achieve a desired effect in a subject being treated with that agent. For example, this can be the amount of a composition comprising one or more Johnson grass pollen allergens (or a biologically active fragment or variant thereof) necessary to reduce, alleviate and/or prevent sensitivity to said allergen. In some embodiments, a “therapeutically effective amount” is sufficient to reduce or eliminate a symptom of sensitivity. In other embodiments, a “therapeutically effective amount” is an amount sufficient to achieve a desired biological effect, for example an amount that is effective to decrease the immune response associated with sensitivity to said Johnson grass pollen allergen.

Ideally, a therapeutically effective amount of an agent is an amount sufficient to induce the desired result without causing a substantial cytotoxic effect in the subject. The effective amount of an agent, for example one or more Johnson grass pollen allergens (or a biologically active fragment or variant thereof), useful for reducing, alleviating and/or preventing inflammation will be dependent on the subject being treated, the type and severity of any associated disease, disorder and/or condition, and the manner of administration of the therapeutic composition.

Suitably, the composition comprises one or more pharmaceutically acceptable carriers, diluents or excipients.

By “pharmaceutically-acceptable carrier, diluent or excipient” is meant a solid or liquid filler, diluent or encapsulating substance that may be safely used in systemic administration. Depending upon the particular route of administration, a variety of carriers, well known in the art may be used. These carriers may be selected from a group including sugars, starches, cellulose and its derivatives, malt, gelatine, talc, calcium sulfate, vegetable oils, synthetic oils, polyols, alginic acid, phosphate buffered solutions, emulsifiers, isotonic saline and salts such as mineral acid salts including hydrochlorides, bromides and sulfates, organic acids such as acetates, propionates and malonates and pyrogen-free water.

A useful reference describing pharmaceutically acceptable carriers, diluents and excipients is Remington's Pharmaceutical Sciences (Mack Publishing Co. N.J. USA, 1991) which is incorporated herein by reference.

A therapeutically effective amount of a composition comprising one or more Johnson grass pollen allergens (or a biologically active fragment or variant thereof) may be administered in a single dose, or in several doses, for example daily, during a course of treatment. However, the frequency of administration is dependent on the preparation applied, the subject being treated, the severity of sensitivity, and the manner of administration of the therapy or composition.

Any safe route of administration may be employed for administering the allergenic protein of the invention. For example, oral, rectal, parenteral, sublingual, buccal, intravenous, intra-articular, intra-muscular, intra-dermal, subcutaneous, inhalational, intraocular, intraperitoneal, intracerebroventricular, transdermal and the like may be employed.

Dosage forms include tablets, dispersions, suspensions, injections, solutions, syrups, troches, capsules, suppositories, aerosols, transdermal patches and the like.

These dosage forms may also include injecting or implanting controlled releasing devices designed specifically for this purpose or other forms of implants modified to act additionally in this fashion. Controlled release of the therapeutic agent may be achieved by coating the same, for example, with hydrophobic polymers including acrylic resins, waxes, higher aliphatic alcohols, polylactic and polyglycolic acids and certain cellulose derivatives such as hydroxypropylmethyl cellulose. In addition, the controlled release may be achieved by using other polymer matrices, liposomes and/or microspheres.

Compositions of the present invention suitable for oral or parenteral administration may be presented as discrete units such as capsules, sachets or tablets each containing a pre-determined amount of one or more therapeutic agents of the invention, as a powder or granules or as a solution or a suspension in an aqueous liquid, a non-aqueous liquid, an oil-in-water emulsion or a water-in-oil liquid emulsion. Such compositions may be prepared by any of the methods of pharmacy but all methods include the step of bringing into association one or more therapeutic agents as described above with the carrier which constitutes one or more necessary ingredients. In general, the compositions are prepared by uniformly and intimately admixing the therapeutic agents of the invention with liquid carriers or finely divided solid carriers or both, and then, if necessary, shaping the product into the desired presentation.

The above compositions may be administered in a manner compatible with the dosage formulation, and in such an amount as is effective to prophylactically and/or therapeutically treat sensitivity to a grass pollen allergen and/or alleviate symptoms associated therewith. The dose administered to a patient, in the context of the present invention, should be sufficient to achieve a beneficial response in a patient over time such as a reduction in the level of circulating allergen-specific IgE, level of sensitivity-related symptoms, or to inhibit allergic or hypersensitive reactions to the grass pollen allergen. The quantity of the therapeutic agent(s) to be administered may depend on the subject to be treated inclusive of the age, sex, weight and general health condition thereof. In this regard, precise amounts of the therapeutic agent(s) required to be administered will depend on the judgement of the clinician. The total dose required for each treatment may be administered by multiple doses or in a single dose.

In determining the effective amount of the therapeutic agent to be administered in the prevention or treatment of sensitivity to a grass pollen allergen, the clinician may evaluate circulating allergen-specific antibody (e.g., of the IgE and/or IgG classes and particularly those of the IgG4 subclass) levels, and/or the response to skin testing and/or any additional diagnostic sensitivity tests outlined above. In any event, suitable dosages of the therapeutic agents of the invention may be readily determined by those skilled in the art. Such dosages may be in the order of nanograms to milligrams of the therapeutic agents of the invention.

In one embodiment, the subject is a human.

In a further embodiment, the therapeutically effective amount of the Johnson grass pollen allergen is administered subcutaneously.

In another embodiment, the therapeutically effective amount of the Johnson grass pollen allergen is administered sublingually.

It is contemplated that the composition may alternatively comprise (i) an isolated nucleic acid, for example, any one or more of SEQ ID NOs: 50 to 89 encoding the isolated protein and/or a recombinant antibody of this aspect, inclusive of variants, derivatives and fragments thereof; (ii) an expression construct encoding the isolated nucleic acid of (i); and/or a host cell comprising the expression construct of (ii).

In one embodiment, the composition further comprises one or more additional environmental allergenic proteins or one or more antibodies which bind or are raised against said allergenic proteins.

Allergens are well known to persons skilled in the art. Common environmental allergens which induce allergic conditions are found in pollen (e.g., tree, herb, weed and grass pollen allergens), food, dust mites, animal hair, dander and/or saliva, moulds, fungal spores and venoms (e.g., from insects) A non-exhaustive list of environmental allergans may be found at the online allergenic molecules (allergens) database, the Allergome (www.allergome.org) or the International Union of Immunological Societies (IUIS) official database of allergens (www.allergen.org).

In particular embodiments, the composition further comprises one or more grass pollen allergens from Bahia grass (Paspalum notatum), Bermuda grass (Cynodon dactylon) and/or Ryegrass (Lolium perenne).

Suitably, the grass pollen allergen/s from Bahia grass may be selected from Pas n 1 and Pas n 13.

Preferably, the grass pollen allergen from Bahia grass is Pas n 1.

Even more preferably, the grass pollen allergen/s from Bahia grass is selected from one or more of those isoforms provided in O'Hehir et al. (WO/2009/052555).

Suitably, the grass pollen allergen/s from Bermuda grass may be selected from Cyn d 1, Cyn d 2, Cyn d 4, Cyn d 6, Cyn d 7, Cyn d 1, Cyn d 12, Cyn d 13, Cyn d 15, Cyn d 22, Cyn d 23 and Cyn d 24.

Preferably, the grass pollen allergen from Bermuda grass is Cyn d 1.

Even more preferably, the grass pollen allergen/s from Bermuda grass is selected from one or more of those isoforms provided in O'Hehir et al. (US 2011/0217325 A1).

Suitably, the grass pollen allergen/s from Ryegrass may be selected from Lol p 1, Lol p 2, Lol p 3, Lol p 4, Lol p 5, Lol p 7, Lol p 10, Lol p 11, Lol p 12 and Lol p 13.

Preferably, the grass pollen allergen from Ryegrass is Lol p 1, Lol p 5 or Lol p 11.

In another embodiment, the composition may be a diagnostic composition suitable for detecting or measuring the level of a Johnson grass allergen disclosed herein, or an immunologically cross-reactive allergen. Suitably, the composition further comprises one or more reagents suitable for diagnostic use. Such reagents may include buffers, diluents, blocking agents, detection reagents and the like, although without limitation thereto. It will also be appreciated that the diagnostic composition may further comprise one or more additional environmental allergens or antibodies thereto, as hereinbefore described.

In another aspect, the invention provides an isolated nucleic acid comprising a nucleotide sequence which encodes, or is complementary to a nucleotide sequence which encodes, an isolated protein comprising an amino acid sequence according to SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, or SEQ ID NO: 43.

In particular embodiments, the isolated nucleic acid comprises, consists of or consists essentially of a nucleotide sequence according to SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88 or SEQ ID NO: 89.

In particular embodiments, the isolated nucleic acid comprises, consists of or consists essentially of a nucleotide sequence set forth in SEQ ID 50, SEQ ID 51 or SEQ ID 52.

This aspect also includes fragments, variants and derivatives of said isolated nucleic acid.

The term “nucleic acid” as used herein designates single- or double-stranded DNA and RNA. DNA includes genomic DNA and cDNA. RNA includes mRNA, RNA, RNAi, siRNA, cRNA and autocatalytic RNA. Nucleic acids may also be DNA-RNA hybrids. A nucleic acid comprises a nucleotide sequence which typically includes nucleotides that comprise an A, G, C, T or U base. However, nucleotide sequences may include other bases such as inosine, methylycytosine, methylinosine, methyladenosine and/or thiouridine, although without limitation thereto.

Accordingly, in particular embodiments, the isolated nucleic acid is cDNA.

In further embodiments, the isolated nucleic acid is codon-optimised nucleic acid.

A “polynucleotide” is a nucleic acid having eighty (80) or more contiguous nucleotides, while an “oligonucleotide” has less than eighty (80) contiguous nucleotides.

A “probe” may be a single or double-stranded oligonucleotide or polynucleotide, suitably labeled for the purpose of detecting complementary sequences in Northern or Southern blotting, for example.

A “primer” is usually a single-stranded oligonucleotide, preferably having 15-50 contiguous nucleotides, which is capable of annealing to a complementary nucleic acid “template” and being extended in a template-dependent fashion by the action of a DNA polymerase such as Taq polymerase, RNA-dependent DNA polymerase or Sequenase™.

Another particular aspect of the invention provides a variant of an isolated nucleic acid that encodes an isolated protein of the invention.

In one embodiment, nucleic acid variants encode a variant of an isolated protein of the invention.

In another embodiment, nucleic acid variants share at least 60% or 65%, 66%, 67%, 68%, 69%, preferably at least 70%, 71%, 72%, 73%, 74% or 75%, more preferably at least 80%, 81%, 82%, 83%, 84%, or 85%, and even more preferably at least 90%, 91%, 92%, 93%, 94%, or 95% nucleotide sequence identity with an isolated nucleic acid of the invention. Percent sequence identity may be determined as previously described.

In yet another embodiment, complementary nucleic acids hybridise to nucleic acids of the invention under high stringency conditions.

“Hybridise and Hybridisation” is used herein to denote the pairing of at least partly complementary nucleotide sequences to produce a DNA-DNA, RNA-RNA or DNA-RNA hybrid. Hybrid sequences comprising complementary nucleotide sequences occur through base-pairing.

“Stringency” as used herein, refers to temperature and ionic strength conditions, and presence or absence of certain organic solvents and/or detergents during hybridisation. The higher the stringency, the higher will be the required level of complementarity between hybridizing nucleotide sequences.

“Stringent conditions” designates those conditions under which only nucleic acid having a high frequency of complementary bases will hybridize.

Stringent conditions are well-known in the art, such as described in Chapters 2.9 and 2.10 of Ausubel et al., supra, which are herein incorporated by reference. A skilled addressee will also recognize that various factors can be manipulated to optimize the specificity of the hybridization. Optimization of the stringency of the final washes can serve to ensure a high degree of hybridization.

Complementary nucleotide sequences may be identified by blotting techniques that include a step whereby nucleotides are immobilized on a matrix (preferably a synthetic membrane such as nitrocellulose), a hybridization step, and a detection step, typically using a labelled probe or other complementary nucleic acid. Southern blotting is used to identify a complementary DNA sequence; Northern blotting is used to identify a complementary RNA sequence. Dot blotting and slot blotting can be used to identify complementary DNA/DNA, DNA/RNA or RNA/RNA polynucleotide sequences. Such techniques are well known by those skilled in the art, and have been described in Ausubel et al., supra, at pages 2.9.1 through 2.9.20. According to such methods, Southern blotting involves separating DNA molecules according to size by gel electrophoresis, transferring the size-separated DNA to a synthetic membrane, and hybridizing the membrane bound DNA to a complementary nucleotide sequence. An alternative blotting step is used when identifying complementary nucleic acids in a cDNA or genomic DNA library, such as through the process of plaque or colony hybridization. Other typical examples of this procedure are described in Chapters 8-12 of Sambrook et al., MOLECULAR CLONING. A Laboratory Manual (Cold Spring Harbor Press, 1989).

Methods for detecting labelled nucleic acids hybridized to an immobilized nucleic acid are well known to practitioners in the art. Such methods include autoradiography, chemiluminescent, fluorescent and colorimetric detection.

Nucleic acids may also be isolated, detected and/or subjected to recombinant DNA technology using nucleic acid sequence amplification techniques.

Suitable nucleic acid amplification techniques are well known to the skilled addressee, and include polymerase chain reaction (PCR); strand displacement amplification (SDA); rolling circle replication (RCR); nucleic acid sequence-based amplification (NASBA), Q-β replicase amplification and helicase-dependent amplification, although without limitation thereto.

As used herein, an “amplification product” refers to a nucleic acid product generated by nucleic acid amplification.

Nucleic acid amplification techniques may include particular quantitative and semi-quantitative techniques such as qPCR, real-time PCR and competitive PCR, as are well known in the art.

In another aspect, the invention provides a genetic construct comprising: (i) the isolated nucleic acid described herein; or (ii) an isolated nucleic acid comprising a nucleotide sequence complementary thereto; operably linked or connected to one or more regulatory sequences in an expression vector.

Suitably, the genetic construct is in the form of; or comprises genetic components of, a plasmid, bacteriophage, a cosmid, a yeast or bacterial artificial chromosome as are well understood in the art. Genetic constructs may be suitable for maintenance and propagation of the isolated nucleic acid in bacteria or other host cells, for manipulation by recombinant DNA technology and/or expression of the nucleic acid or an encoded protein of the invention.

For the purposes of host cell expression, the genetic construct is an expression construct. Suitably, the expression construct comprises the nucleic acid of the invention operably linked to one or more additional sequences in an expression vector. An “expression vector” may be either a self-replicating extra-chromosomal vector such as a plasmid, or a vector that integrates into a host genome. Non-limiting examples of expression constructs include adenovirus vectors, adeno-associated virus vectors, herpesviral vectors, retroviral vectors, lentiviral vectors, and the like. For example, adenovirus vectors can be first, second, third, and/or fourth generation adenoviral vectors or gutless adenoviral vectors. Adenovirus vectors can be generated to very high titers of infectious particles, infect a great variety of cells, efficiently transfer genes to cells that are not dividing, and are seldom integrated in the host genome, which avoids the risk of cellular transformation by insertional mutagenesis (Douglas and Curiel, Science and Medicine, March/April 1997, pages 44-53; Zern and Kresinam, Hepatology 25:484-91, 1997). Representative adenoviral vectors are described by Stratford-Perricaudet et al. (J. Clin. Invest. 90:626-30, 1992), Graham and Prevec (In Methods in Molecular Biology: Gene Transfer and Expression Protocols 7:109-28, 1991) and Barr et al. (Gene Therapy, 2:151-55, 1995).

Adeno-associated virus (AAV) vectors also are suitable for administration of the nucleic acids of the invention. Methods of generating AAV vectors, administration of AAV vectors and their uses are well known in the art (see, e.g., U.S. Pat. No. 6,951,753; U.S. Patent Application Publication Nos. 2007/036757, 2006/205079, 2005/163756, 2005/002908; and PCT Publication Nos. WO 2005/116224 and WO 2006/119458).

By “operably linked” is meant that said additional nucleotide sequence(s) is/are positioned relative to the nucleic acid of the invention preferably to initiate, regulate or otherwise control transcription.

Regulatory nucleotide sequences will generally be appropriate for the host cell used for expression. Numerous types of appropriate expression vectors and suitable regulatory sequences are known in the art for a variety of host cells.

Typically, said one or more regulatory nucleotide sequences may include, but are not limited to, promoter sequences, leader or signal sequences, ribosomal binding sites, transcriptional start and termination sequences, translational start and termination sequences, and enhancer or activator sequences.

Constitutive or inducible promoters as known in the art are contemplated by the invention. The promoters may be either naturally occurring promoters, or hybrid promoters that combine elements of more than one promoter. Non-limiting examples of promoters include SV40, cytomegalovirus (CMV), and HIV-1 LTR promoters.

The expression construct may also include an additional nucleotide sequence encoding a fusion partner (typically provided by the expression vector) so that the recombinant allergenic protein of the invention is expressed as a fusion protein, as hereinbefore described.

In a further aspect, the invention provides a host cell transformed with a nucleic acid molecule or a genetic construct described herein.

Suitable host cells for expression may be prokaryotic or cukaryotic. For example, suitable host cells may be mammalian cells (e.g. HeLa, HEK293T, Jurkat cells), yeast cells (e.g. Saccharomyces cerevisiae), insect cells (e.g. Sf9, Trichoplusia ni) utilized with or without a baculovirus expression system, or bacterial cells, such as E. coli, or a Vaccinia virus host. Introduction of genetic constructs into host cells (whether prokaryotic or eukaryotic) is well known in the art, as for example described in CURRENT PROTOCOLS IN MOLECULAR BIOLOGY Eds. Ausubel et al., (John Wiley & Sons, Inc. 1995-2009), in particular Chapters 9 and 16.

In yet another aspect, the invention provides a method of producing a recombinant protein described herein, comprising; (i) culturing the previously transformed host cell hereinbefore described; and (ii) isolating said protein from said host cell cultured in step (i).

The recombinant protein may be conveniently prepared by a person skilled in the art using standard protocols as for example described in Sambrook, et al., MOLECULAR CLONING. A Laboratory Manual (Cold Spring Harbor Press, 1989), in particular Sections 16 and 17; CURRENT PROTOCOLS IN MOLECULAR BIOLOGY Eds. Ausubel et al., (John Wiley & Sons, Inc. 1995-2009), in particular Chapters 10 and 16; and CURRENT PROTOCOLS IN PROTEIN SCIENCE Eds. Coligan et al., (John Wiley & Sons, Inc. 1995-2009), in particular Chapters 1, 5 and 6.

In another further aspect, the invention provides a diagnostic and/or screening kit comprising: (i) one or more of the proteins described herein and/or one or more antibodies that bind or are raised against the proteins; and (ii) instructions for use.

This aspect also includes fragments, variants and derivatives of said proteins and/or antibodies that bind to or are raised against said isolated protein, variant or derivative.

It would be appreciated that certain embodiments of this aspect may be used for detecting and/or monitoring sensitivity to one or more Johnson grass pollen allergens in a subject. Further embodiments of this aspect may be used in detecting and/or monitoring the presence of one or more Johnson grass pollen allergens in the environment. Even further embodiments of this aspect may be used in measuring levels of one or more Johnson grass pollen allergens in a therapeutic or diagnostic sample for batch standardization.

In one embodiment, the kit further comprises one or more additional environmental allergens or antibodies thereto.

Accordingly, the kit of this aspect of the invention may comprise two or more different allergens originating from, and/or antibodies thereto, the same allergenic grass, such as Sor h 1 (i.e., SEQ ID NOs: 1 or 2) and Sor h 13 (i.e., SEQ ID NOs: 3, 4, 5 or 6), and/or from different allergenic grasses, such as Sor h 1 (i.e., SEQ ID NOs: 1 or 2) and Pas n 1, and/or even different allergenic sources, such as Sor h 1 (i.e., SEQ ID NOs: 1 or 2) and the dust mite allergen, Der p 1. Furthermore, more than one isoform, and/or antibodies directed to more than isoform, of the same allergen may be included in the kit of this aspect.

The allergen of this aspect may be a purified allergen, a recombinant allergen or it may be in the form of a crude allergen extract.

The allergen protein or antibody of the kit may be provided in a composition, such as a diagnostic composition as hereinbefore described. The kit may further comprise additional diagnostic reagents such as secondary antibodies, enzymes (e.g., alkaline phosphatase or horseradish peroxidase) and/or substrates for the enzymes (e.g., Luminol, ABTS or NBT). The antibody and/or the secondary antibody may be labeled as hereinbefore described.

In a further aspect, the invention provides a method of determining the amino acid sequence of a grass pollen allergen, including the steps of: (i) preparing cDNA from RNA extracted from a grass pollen; (ii) determining the nucleotide sequence of said cDNA library; (iii) isolating allergenic proteins or fragments thereof from the corresponding grass pollen in (i); (iv) determining the amino acid sequence of the isolated allergen proteins or fragments thereof from (iii).

Preferably, the method further comprises extracting RNA from a grass pollen and preparing an RNA fragment library from said RNA.

Preferably, the method further includes the step of confirming the amino acid sequence of (iii) by aligning and comparing the predicted peptide sequence encoding the nucleotide sequence of (ii) with the amino acid sequence of (iii).

It will be appreciated that the method adopted by the current invention has been successfully used to identify a number of previously unknown Johnson grass pollen allergens. Accordingly, this method provides a novel means of creating a grass pollen allergome through modern transcriptome-proteome assembly and analysis techniques.

In order that the invention may be readily understood and put into practical effect, particular preferred embodiments will now be described by way of the following non-limiting examples.

### Example 1

### Materials & Methods

Clinical Study Participants.

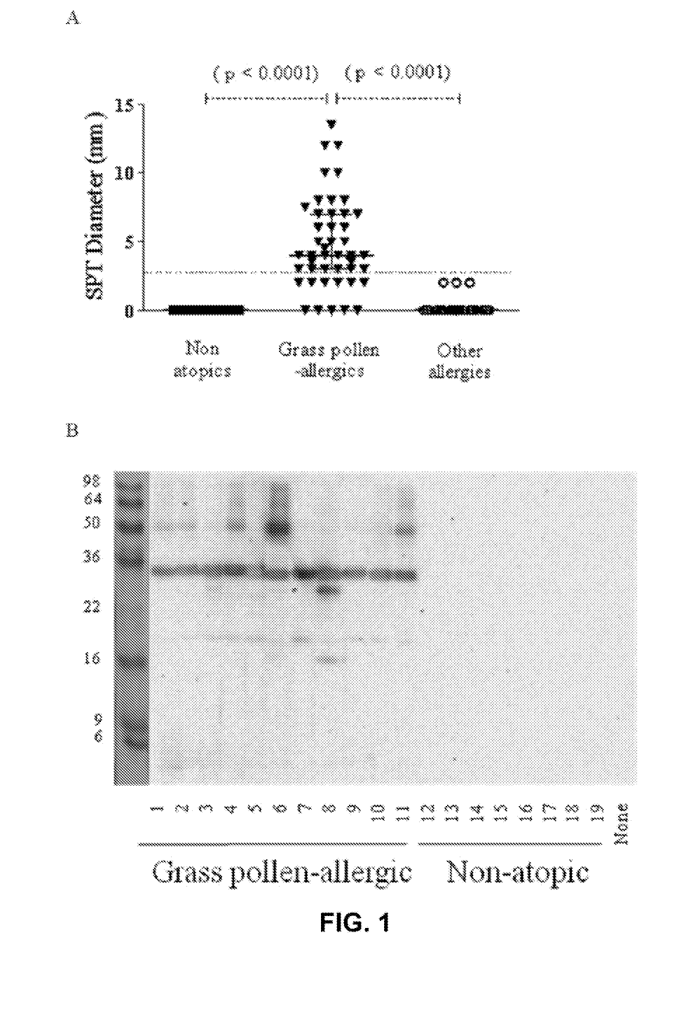

Participants were recruited from immunology or respiratory clinics at The Princess Alexandra Hospital, Brisbane, or regional parts of Queensland, Australia, with informed consent as approved by the Metro South Human Research Ethics Committee. Subjects were tested for allergic sensitivity to a panel of 10 common aeroallergen extracts including Johnson, Bahia, Bermuda or Ryegrass pollen extracts by skin prick test (SPT) (Hollister-Stier, USA) according to guidelines of the Australian Society for Clinical Immunology and Allergy (FIG. 1A). Diameters greater than 3 mm were considered positive. The grass pollen-allergic patients had a history of allergic rhinitis consistent with pollen allergy and showed a SPT response to the pollen extract of at least one grass species (n 64). Non-atopic subjects with no history of allergic disease and no positive SPT response (n=19), and subjects with histories of allergic rhinitis and asthma with SPT responses to allergens other than grass pollens, frequently house dust mite, cat dander or Alternaria, were included as controls (n=23). Sera were obtained from participants by venepuncture.

One and Two Dimensional Gel Electrophoresis and Immunoblotting.

JGP (Greer, Lenois USA) extracted in phosphate buffered saline was separated by 14% SDS PAGE gel electrophoresis (10 μg per lane) and immunoblotted for monoclonal antibody (mA) or serum IgE reactivity using the following modifications to published methods (Davies et al., Mol Immunol, 2011). Patient sera diluted 1/50 were incubated overnight before incubation with rabbit anti-human IgE diluted 1/10,000 for 2 hours and goat anti-rabbit IgG-horse radish peroxidase conjugate at 1/10,000 for 2 hours. IgB immunoblots were developed for 5 minutes by chemiluminescence (Pierce). Immunoblots probed with mAb 6C6 (Davies et al., Clin Exp Allergy, 2011) and AF6 (Petersen et al., Proteomics, 2006) were visualized by standard 1,4-dichloronapthol development (Davies et al., Clin Exp Allergy, 2011). JGP (50 μg per dry gel strip, pH 3-11, GE Healthcare, Uppsala Sweden) was separated by charge and size by two dimensional (2D) gel electrophoresis and stained with Coomassie Brilliant Blue as described in Davies et al., Mol Immunol, 2011. 2D gels of JGP were also immunoblotted and probed for IgE reactivity with serum pools of 11 JGP-allergic donors and 8 non-atopic donors, or mAb reactivity as described above. 2D gels of JGP spiked with isoelectric focusing standard proteins were examined to determine the observed molecular weights and isoelectric focusing points of IgE reactive components.

Serum IgE reactivity with purified Sor h 1 and Sor h 13.

The two dominant allergenic components of JGP were purified from an aqueous extract of JGP by ammonium sulphate precipitation, hydrophobic interaction and size exclusion chromatography as described for Pas n 1 (Drew et al., Int Arch Allergy Immunol, 2011) and Pas n 13 (Davies et al., Clin Exp Allergy, 2011) of Bahia grass pollen. Sera from 19 non-atopic donors, 23 donors with allergic sensitivities to allergens other than grass pollen (other allergies) and 64 grass pollen-allergic patients, including 31 recruited from regional parts of QLD, were tested for serum IgE reactivity with whole JGP extract (5 μg/ml) and the purified allergens (1 μg/ml) by ELISA (Davies et al., Clin Exp Allergy, 2011).

Statistical Analysis.

The distribution of data was assessed for normality by Kolmogorov-Smirnov test. Statistical differences between groups were assessed by Mann Whitney U test for non-parametrically distributed data. Within group differences in responses to allergens were assessed by Wilcoxon signed ranks test for paired data. Correlations of IgE reactivity with JGP compared with each purified allergen were determined by Spearman's rank test for paired data. P values less than 0.05 were considered significant.

### Results

Allergenic Components of Johnson Grass Pollen for Patients from Subtropical Region.

By immunoblotting, sera of 11 grass pollen-allergic patients from Queensland with positive SPT to JGP, showed IgE reactivity with a 30 kDa component consistent in size with the known group 1 allergen, Sor h 1 (FIG. 1B). Five of these 11 patients showed IgE reactivity with a protein component at 55 kDa. JGP-allergic patients also showed IgE reactivity with other allergenic components of JGP; bands at 28, 18 and 16 kDa reacted with 3, 11 and 1 sera respectively (FIG. 1B). Eight non-atopic participants showed no IgE with any JGP components.

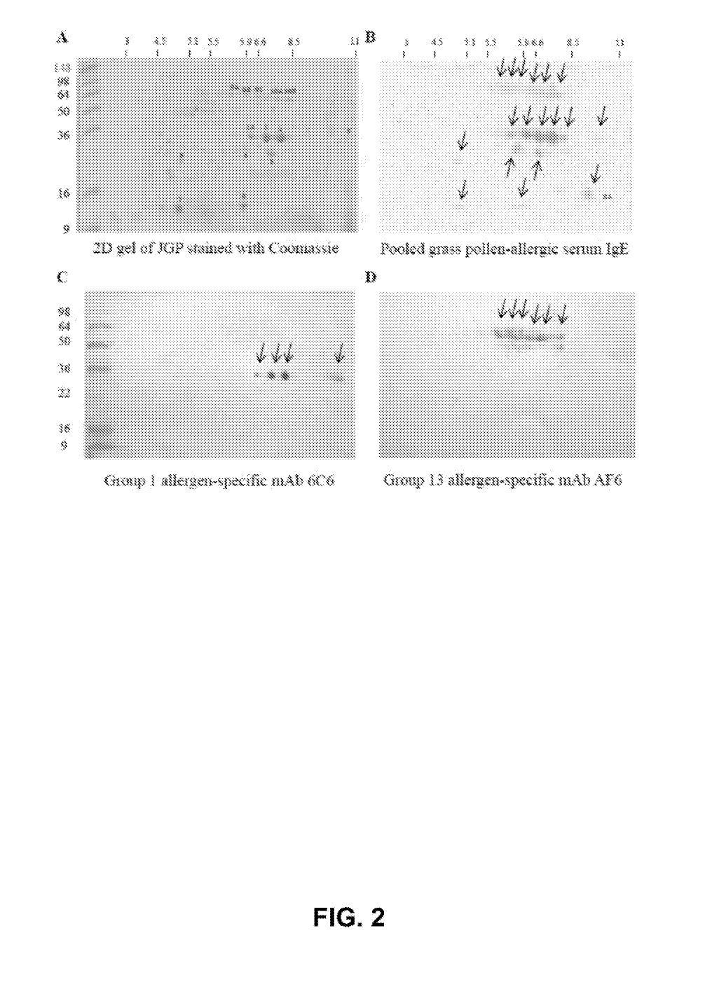

Forty seven protein components of JPG were evident by 2D gel electrophoresis (FIG. 2A). By 2D immunoblotting, 18 spots showed IgE reactivity with serum pooled from the 11 JGP allergic patients (FIG. 2B), but no IgE reactivity was observed with serum pooled from the eight non-atopic donors in FIG. 1 (data not shown). Three protein spots with neutral pI (63, 6.8 and 7.1) at 30 kDa reacted with patient IgE and with a mAb 6C6 to the group 1 allergen of Bermuda grass pollen (Cyn d 1), confirming the identity of these 30 kDa allergenic components as isoforms of Sor h 1. An additional basic isoform of Sor h 1 was present at a low amount (FIG. 2A) but showed reactivity with the 6C6 mAb (FIG. 2C) and weak IgE reactivity (FIG. 2B). Six spots with pIs from 5.7 to 7.6 at 54-55 kDa were IgE reactive with the JGP-allergic serum pool (FIG. 2B) and with the mAb AF6 to the group 13 allergen of Timothy grass pollen (Phl p 13) (FIG. 2D). The 55 kDa allergenic component of JGP was designated as Sor h 13. IgE-reactive spots at 28 kDa, 26 kDa, 18 kDa and two at 16 kDa were observed (Table 2).

Serum IgE reactivity with dominant allergenic components of JGP.

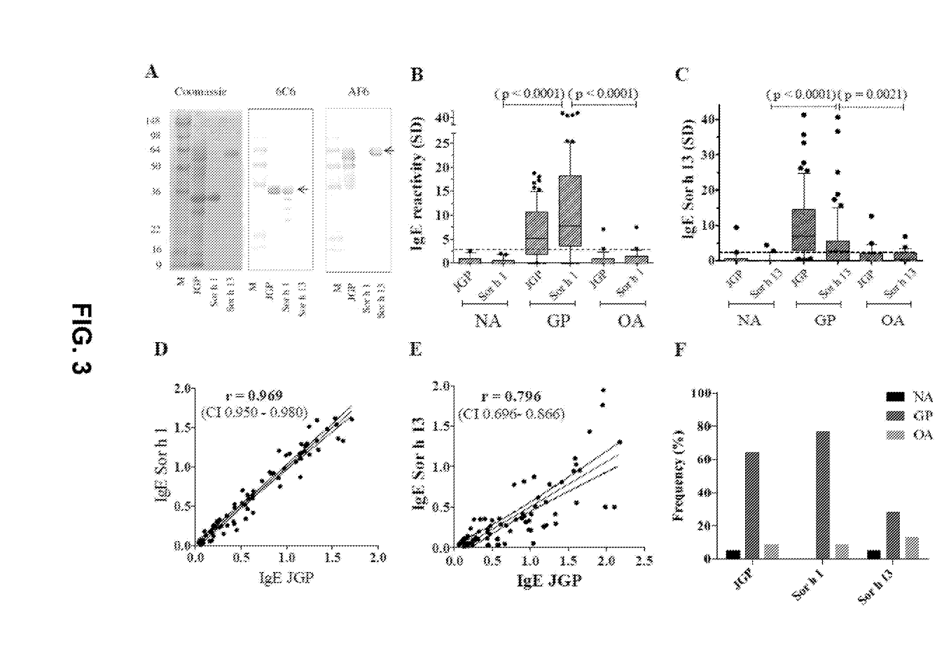

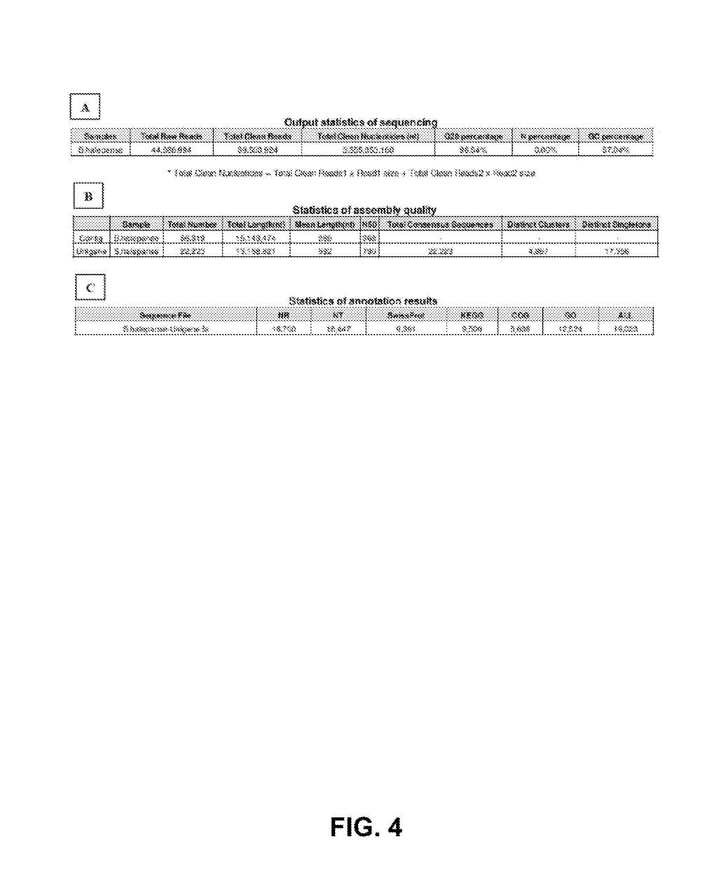

The dominant allergenic components of JGP, Sor h 1 and Sor h 13 were purified to a single protein band and their identity was confirmed by immunoblotting with allergen-specific mAb (FIG. 3A). Scrum IgE reactivity with JGP and purified Sor h 1 and Sor h 13 allergens was assessed in 19 non-atopic donors, 23 donors with allergic sensitivities to allergens other than grass pollen and 64 grass pollen-allergic patients from a subtropical region. Since there was significantly lower level of IgE reactivity amongst the non-atopic donors with purified Sor h 1 compared with JGP (FIG. 4; p=0.0085), and because samples were assayed across multiple days, the data for each donor were expressed as the number of standard deviations above the mean of the non-atopic donors of whom there were at least 12 included in each assay.

There was significantly higher serum IgE reactivity with Sor h 1 in the JGP allergic subject group than the non-atopic and other allergy control groups (FIG. 3B). IgE reactivity with JGP and Sor h 1 were highly correlated (r=0.969) (FIG. 3D). There was a higher level of IgE reactivity with Sor h 1 in JGP-allergic patients than in patients with other allergies or non-atopic control donors (Wilcoxon rank-signed test, p<0.0001). Whereas 41 of 64 grass pollen-allergic donors showed serum IgE reactivity with JGP (64%), 49 patients showed IgE reactivity with Sor h 1 (76.5%, FIG. 3F), consistent with the frequency of 77% of subjects who showed SPT reactivity with JGP amongst the grass pollen-allergic patients). Of the 41 grass pollen-allergic donors with positive SPT to JGP, 40 (97.5%) showed IgE reactivity with Sor h 1.

Serum IgE with Sor h 13 was detected in 28 of the 64 (43.7%) of grass pollen allergic donors by ELISA (FIGS. 3C & F). IgE reactivity with Sor h 13 was significantly higher in the grass pollen-allergic patients than non-atopic and other allergy control groups (FIG. 3C) (Wilcoxon, p<0.0001). There was a strong correlation between IgE reactivity with Sor h 13 and JGP (r=0.796) (FIG. 3E). There was one non-atopic donor and three patients with other allergies who showed serum IgE reactivity with Sor h 13 (FIG. 3F).

The inventors have further developed an ImmunoCAP® (Pharmacia diagnostics) assay for the measurement and detection of specific IgE to the JGP allergens Sor h 1 and Sor h 13 which has potential utility for the diagnosis of patients with grass pollen allergy. An ImmunoCAP test is considered the gold standard for the detection and/or measurement of IgE antibodies to specific allegens as it performs excellently for IgE antibody detection as well as enabling quantitative measurements thereof.

As would be appreciated by the skilled artisan, an ImmunoCAP test first requires the covalent coupling, such as by streptavidin and biotin, of the allergen of interest to a cellulose-based solid phase. A biological sample from the patient, typically serum or plasma, is then contacted with this solid phase, such that the allergen of interest can react and bind with any corresponding IgE in the patient's sample. After suitable incubation, any unbound IgE is then washed away and enzyme-labelled anti-IgE antibodies are added. Following suitable incubation, any unbound enzyme-anti-IgE is washed away and the ImmunoCAP is incubated with a suitable developing agent. The fluorescence of the eluate is then measured following quenching of the enzyme-based reaction. An IgE level in the patient's sample can then be determined by comparing the result of the test to a reference curve or samples of known IgE concentrations.

### Example 2

### Materials and Methods

Transcriptome sequencing of Johnson Grass Pollen.

Total RNA was extracted from mature pollen grains of Johnson grass pollen utilising a modified protocol based on Li and Trick 2005 (Li and Trick, Biotechniques, 2005). Total RNA was DNase treated with the Ambion® TURBO™ DNase kit according to manufacturer's instruction. RNA quality was visualised on an agarose gel and confirmed using an Agilent 2100 Bioanalyzer (Santa Clara, Calif., USA). The RNA Integrity Number value was 8.7. The concentration of RNA was measured using a NanoDrop 8000 Multi-Sample Micro-Volume UV-Vis Spectrophotometer (Thermo Fisher Scientific, Wilmington Del., USA). The cDNA library preparation and sequencing was completed by Beijing Genomics Institute (BGI), Shenzen, China using the RNA-seq pipeline from Illumina (www.illumina.com).

Transcriptome Data Analysis.

De novo transcriptome assembly was carried out with the short reads assembly program—Trinity (Grabherr et al., Nat Biotechnol, 2011). Once assembled, a blastx alignment (evalue <0.00001) between Unigenes and protein databases NCBI-nr, Swiss-Prot, KEGG and COO was performed. The results with the best alignment scores were used to inform Unigene sequence direction and functional annotation. Conflicting database results were resolved using the priority order of nr, Swiss-Prot, KEGG and COG when deciding sequence direction of Unigenes. When a Unigene could not be aligned to any of the above databases, ESTScan was utilized (Iseli et al., Proc Int Conf Intell Syst Mol Biol, 1999). Gene abundance was calculated using RSEM v1.2.0 (Li and Dewey, BMC Bioinformatics, 2011).

A set of predicted peptide sequences were constructed from the total JGP messenger RNA transcriptome assembly translated in all six frames by sequentially running the total JGP transcriptome library through the Sequence Manipulation Suite (SMS; http://www.bioinformatics.org/sms2/translate.html) and selecting for each reading frame using the standard translation code. The predicted proteome of JGP comprising a concatenated file containing all six frames of possible peptides was then compared to the grass pollen allergen protein sequences in Allergome (Allergome.org), a comprehensive database of up to 6896 allergens, by BlastP.

Proteome Assembly of Johnson Grass Pollen by Mass Spectrometry (MS).

In-gel digest was performed as previously described by Davies et al. (Mol Immunol, 2011) with the difference that the in-gel digest was conducted on ID gel slices containing whole JGP extracted in PBS or purified allergen resolved over 8 mm. Tryptic peptides were separated and analysed with Agilent's 1200 HPLC Chip cube coupled to the 6520 QTOF. A flow rate of 4 μL/min was used to load the peptides onto the enrichment column of a Large Capacity HPLC Chip (Agilent G4240-62010) and a flow rate of 0.3 ul/min was used to separate the peptides on the analytical column with a 5-50% buffer B gradient in 45 min. The HPLC chip was cleaned with 95% buffer B for 9 mins and equilibrated with buffer 5% B for 9 mins. The HPLC gradient used Buffer A with 0.1% formic acid and buffer B with 0.1% formic acid, 90% acetonitrile. Mass spectrum acquisition was set to 8 MS and 4 MS/MS per second. Dynamic exclusion was applied after 2 precursor spectra and released after 0.25 min. The observed peptides were searched against a database of all six possible translation frames of putative peptide sequences deduced from the JGP transcriptome using Spectrum Mill (Agilent B.04.00.127). The parameters used in Spectrum Mill are detailed in Davies et al. (Mol Immunol, 2011).

In the absence of knowledge of the Johnson grass genome from which to predict peptide sizes, the mass spectra of tryptic digest peptide of the total JGP extract were compared against the NBCI non-redundant plant database, the predicted peptide library of transcripts generated using ORFPredictor and a database created from all six reading frames of the JGP transcriptome library. Mass spectra of tryptic digest peptides from purified Sor h 13 were analysed against the predicted peptide library of transcripts generated using ORFPredictor. Tryptic digest peptide fragments observed in excised IgE reactive spots were compared against all possible peptides predicted from all six reading frames of the transcriptome assembly.

The coverage of peptide spectra from the IgE-reactive spots were mapped against the predicted protein sequences from the relevant reading frame using Geneious (www.biomatters.com). Signal peptides were predicted using the SignalP 4.1 online tool (http//www.cbs.dtu.dk/services/SignalP/) and if present these signals were annotated on the predicted protein. Peptide spectra were mapped to the predicted peptide sequence for which the highest number of unique matches were observed. Peptide spectra were aligned to multiple sequences when the specific origin of a peptide could not be determined. Where peptides were compared to multiple predicted proteins, alignments were performed using MUSCLE in the Geneious environment with standard parameters. Molecular mass and pI of predicted peptides from the JGP transcriptome were performed using ExPASy proteomic tools (www.expasy.org). Signal peptides were not included in alignments or calculations of pI and molecular mass.

### Results

Quality of JGP Transcriptome Sequencing.

Sequencing of the JGP transcriptome [Sorghum halepense (L.) Pers, 2n=2x=40], yielded a total of 44, 686, 994 raw and 39, 503, 924 clean reads with a Q20 quality score of 96.54% (FIG. 4). Transcriptome assembly identified 56, 319 contigs and 22, 223 Unigenes (FIG. 4).

Identification of Allergens within the Proteome and Transcriptome of JGP.

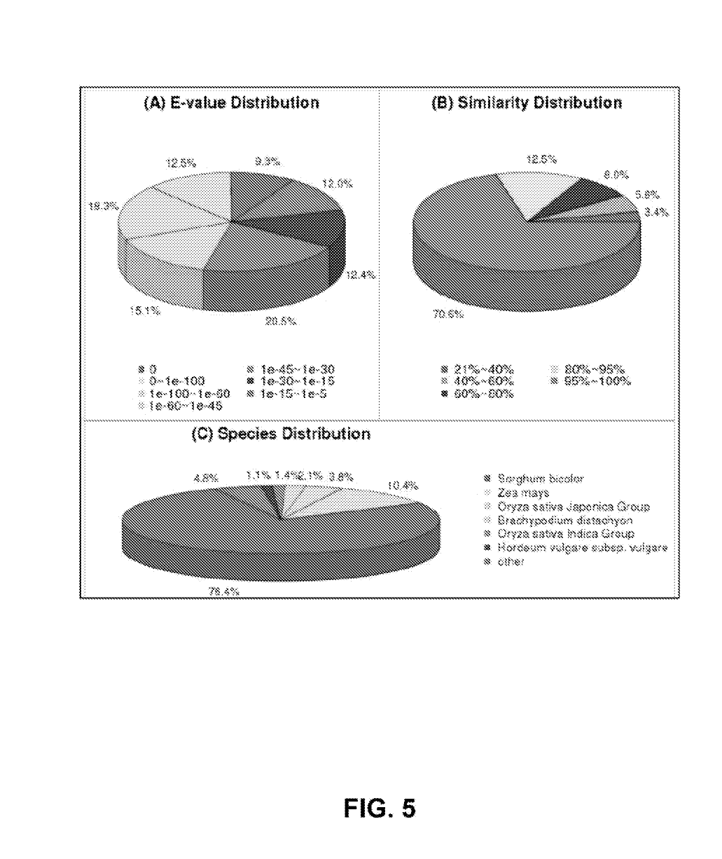

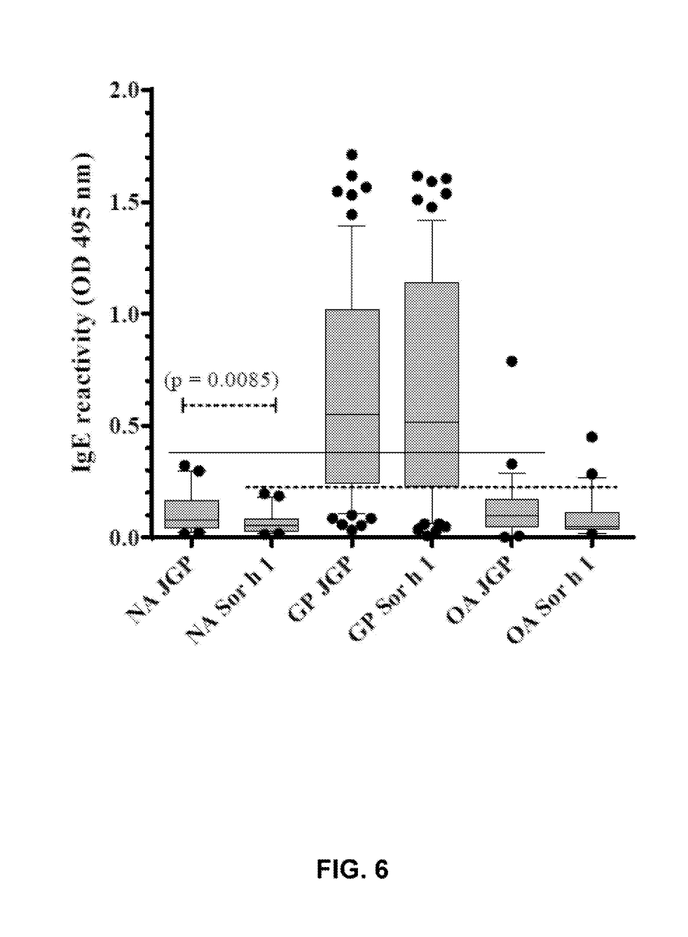

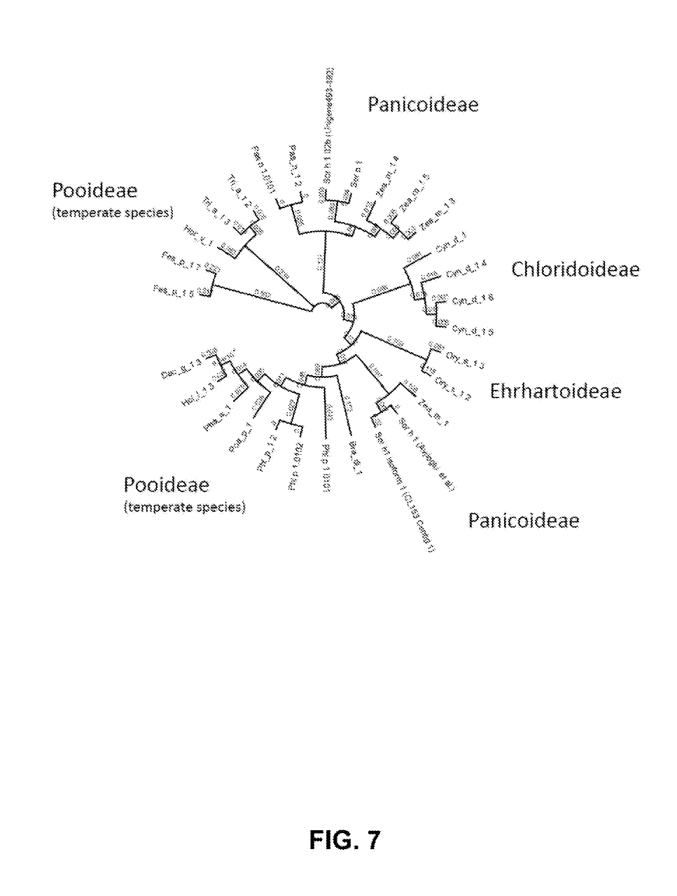

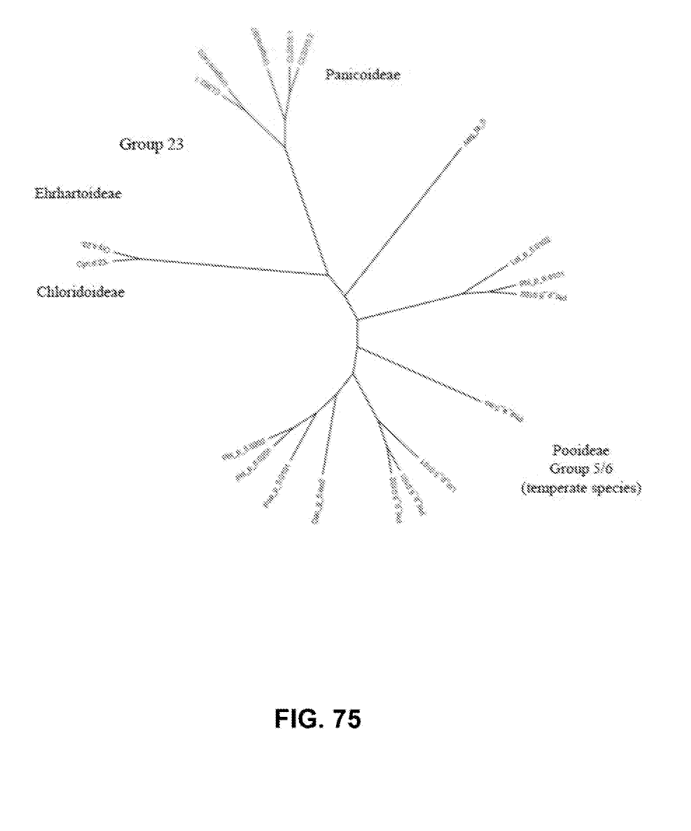

To identify the additional molecular allergenic components the total JGP pollen transcriptome was sequenced revealing high quality sequence data for expressed RNA originating from over 22 thousand potential gene candidates (FIG. 5). The JGP transcriptome had 76.4% sequence identity with the closely related species S. bicolor, 10.4% with Zea mays and 8.6% with Oyza sativa (FIG. 5). Tryptic digestion of total JGP revealed 4609 peptide spectra observed by mass spectrometry that matched the predicted proteome of JGP based on the total pollen transcriptome (Table 3). Subsequently, the potential allergome of S. halepense was deduced by BLAST results against the IUIS official list of allergens (www.allergen.org/), revealing up to 685 unique hits against a database of approximately 1800 known allergens (Table 3). A full listing of Johnson grass allergens identified so far are provided in FIGS. 7-75 and SEQ ID Nos: 1-49. Encoding nucleic acids are SEQ ID Nos. 50-89. Some of the key allergen groups identified in JGP matched pollen allergens of the temperate grasses including timothy (Phleum pratense) Phl p 1, 2, 3, 4, 7, 11, 12 and 13; and ryegrass (Lolium perenne) Lol p 1, 2, 3, 4, and 11; as well as the subtropical grasses Bermuda (Cynodon dactylon) Cyn d 1, 2, 4, 11, 12, 15, 22 and 23; and Bahia (Paspalum noratum) Pas n 1 and 13. The allergen groups most notably missing were Phl p 5 and 6, important allergens of temperate grasses (Table 1). The putative pollen allergens of JGP based on their presence in the transcriptome and proteome of Johnson grass pollen and the Allergome.org database are listed in Table 4.

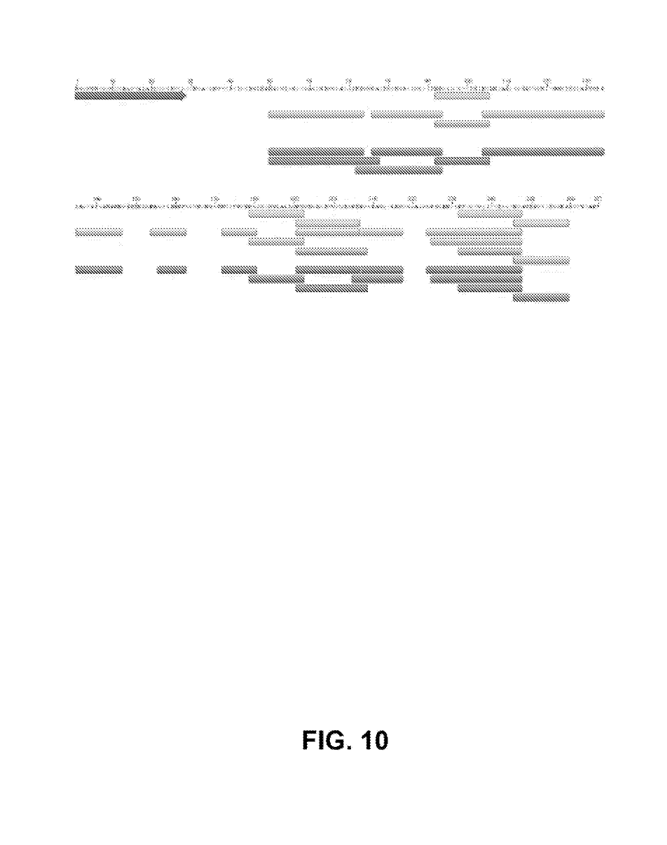

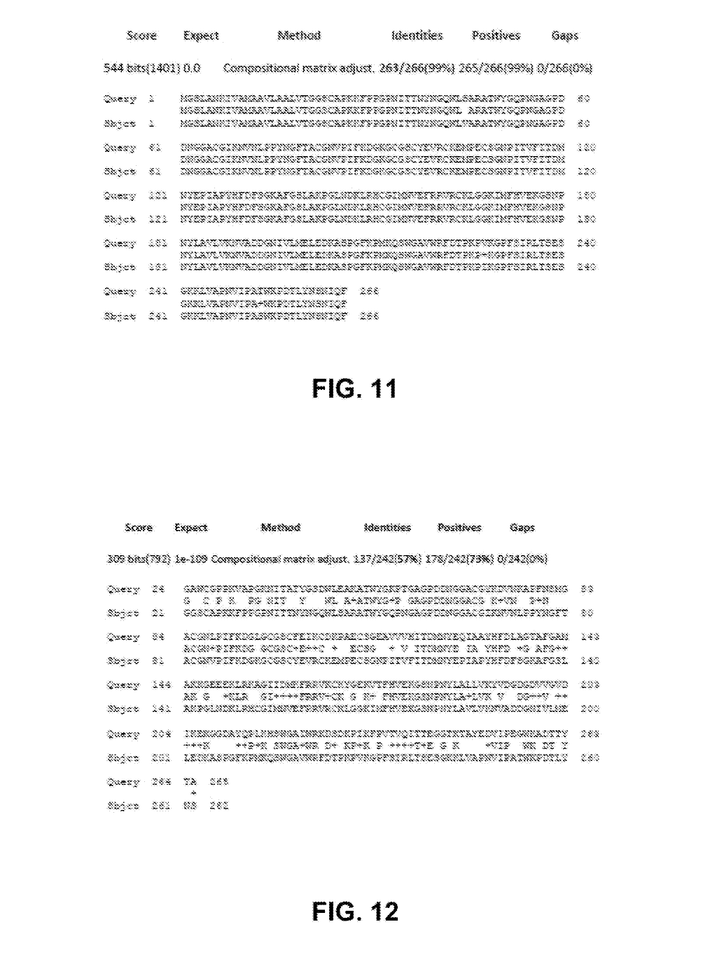

There was 99% sequence identity between CL153 isoform 1 with the previously published Sor h 1 sequence (Avjioglu et al., Molecular Biology and Immunology of Allergens, 1993), validating the experimental strategy of combining transcriptomic and proteomic data to characterise an allergome in the absence of genomic data. MS analysis showed 78% coverage of the contig CL153 (FIG. 10). The spectra of unique peptides for the IgE reactive protein spots 1 and 2 matched this contig. (Table 2).

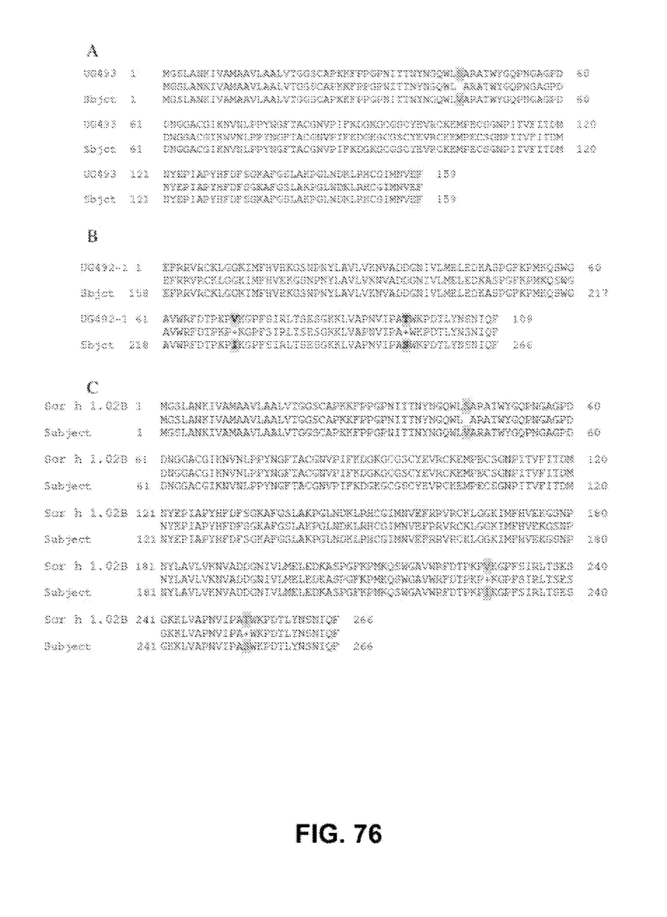

Transcripts for Sor h 1, 2 and 15 show homology to genes belonging to the p-expansin family of proteins, based on BLAST results and identified functional domains (Tables 1 and 3). Furthermore, the observed isoelectric points and molecular weights from the excised IgE-reactive protein spots approximately matched their published equivalent in other species. This was the case with all other allergen groups identified. The clustering pattern of group 1 allergens showed that sub-tropical species formed a distinct clade from the temperate (FIG. 7). Two of the transcripts encoding Sor h 1 (contigs CL153, 1 and 2), only differed within the translation start site. A second group 1 allergen isoform designated Sor h 1.02B (FIG. 7), was encoded by concatenation of two overlapping transcripts UG 493 and UG 492 (FIG. 76). These Sor h 1.01A and Sor h 1.02B isoforms are likely to be encoded by separate loci given that their charges (pI) differ (Table 2) and their predicted peptide sequences share only 57% amino acid identity and 73% similarity, respectively (FIG. 12). Moreover, these two isoforms aligned to separate branches of a dendrogram of group 1 grass pollen allergens (FIG. 7).

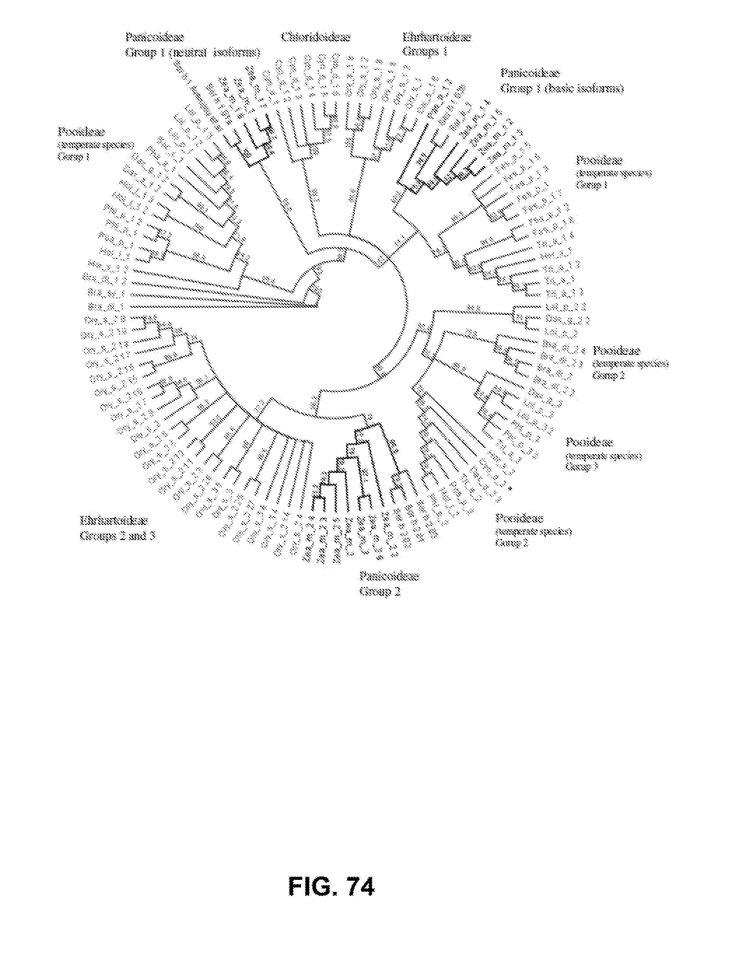

Based on the predicted pI of deduced peptides and spectra of peptide of IgE-reactive spots contigs CL1122.1 and CL1695.1, encode proteins consistent with Sor h 2 (Tables 1 & 2). The contig CL1122.2 encodes a peptide predicted to have basic pI of 9.35 more consistent with group 3 allergens (Table 1), but it also aligns closely with group 2 allergens (FIG. 74).

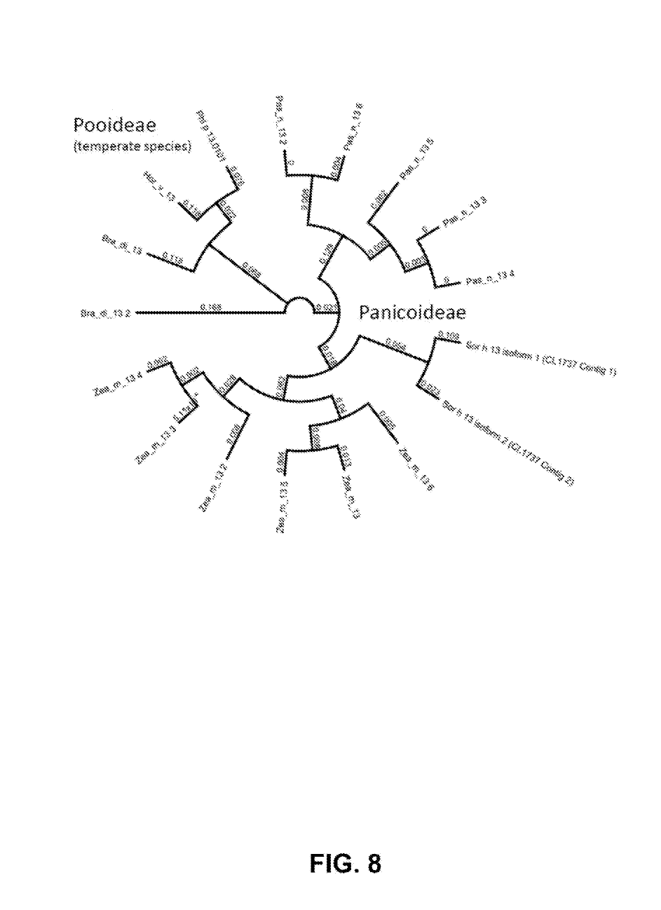

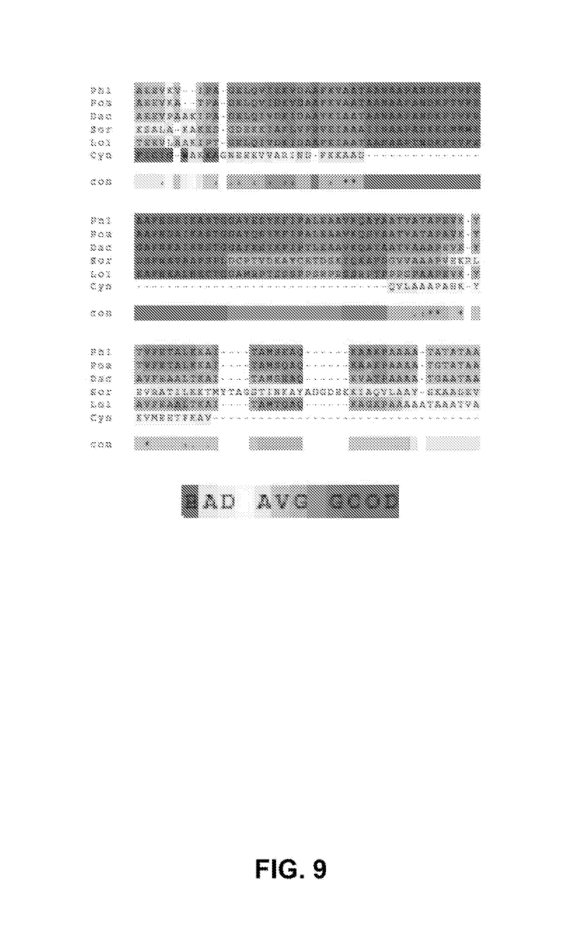

Contig CL1737.1 and CL1737.2 encode related proteins with predicted MW and pI of 41.6 kDa, pI of 6.59 and of 40.5 kDa, pI 7.84 consistent with group 13 allergen isoforms designated Sor h 13.01 and Sor h 13.02. The three predicted asparagine glycosylation sites in both sequences could account for the discrepancy in predicted and observed size. BLAST analysis and sequence alignments showed contig CL1737.1 and CL1737.2 had 76% homology to Phl p 13 (CAB42886.1) and had the functional domains of a polygalacturonase (Table 1). The gene tree for the group 13 allergens illustrated how the Sor h 13 sequences fall into the same clade as sorghum's close relative Zea mays (FIG. 8). Sor h 13.1 and Sor h 13.2 showed 86% identity and 88% similarity in peptide sequence with most divergence in the signal peptide and amino-terminal.

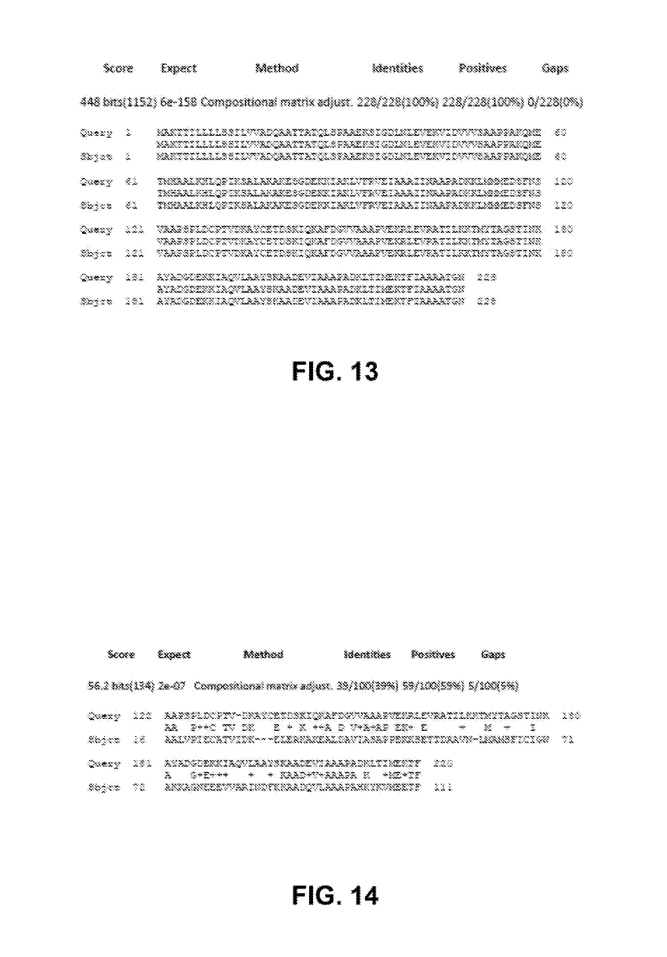

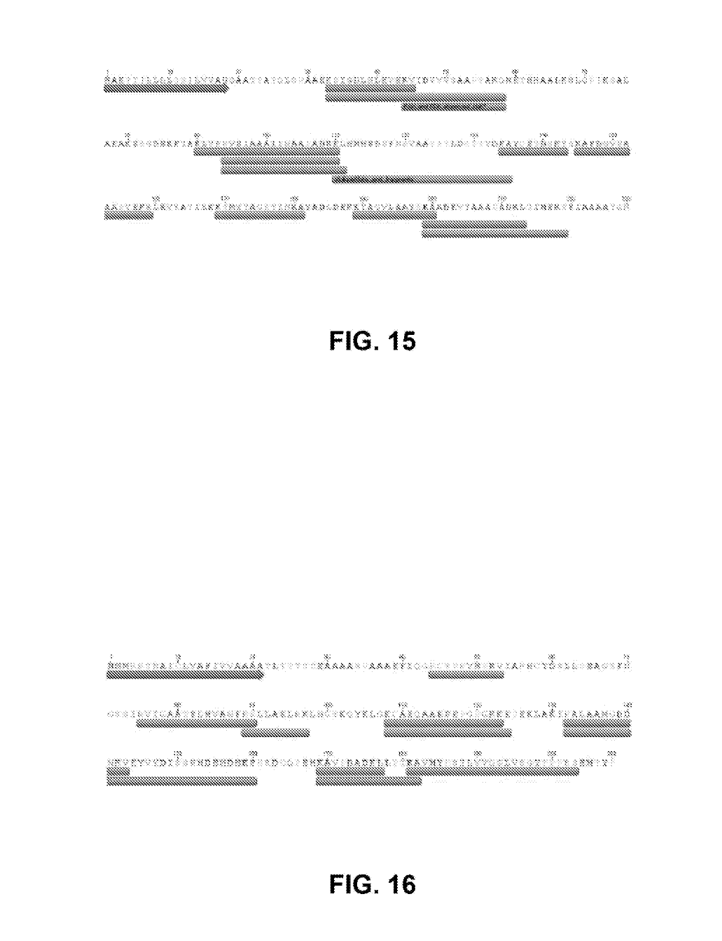

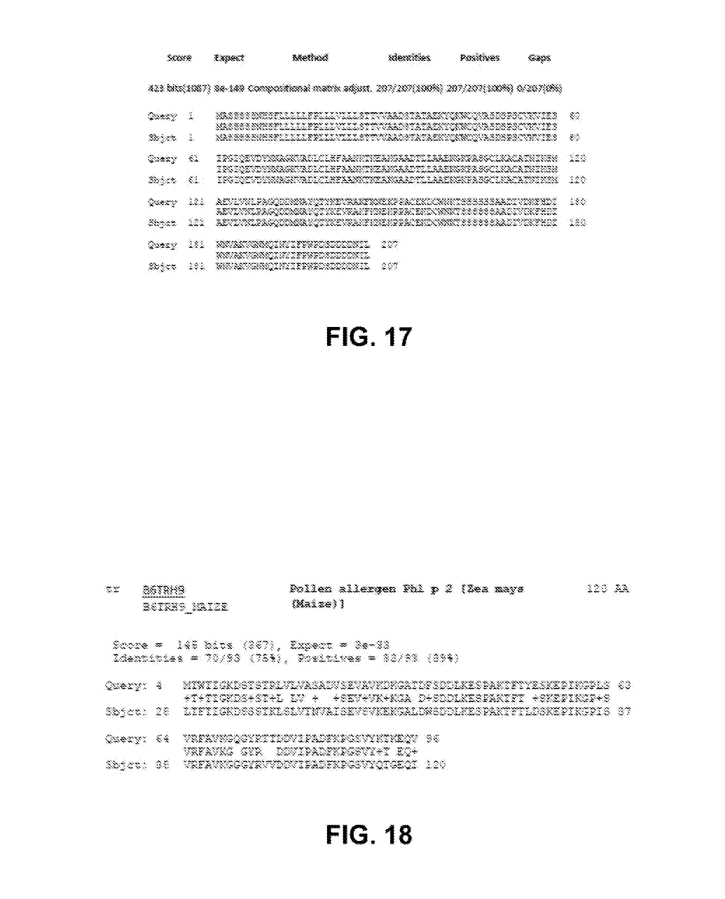

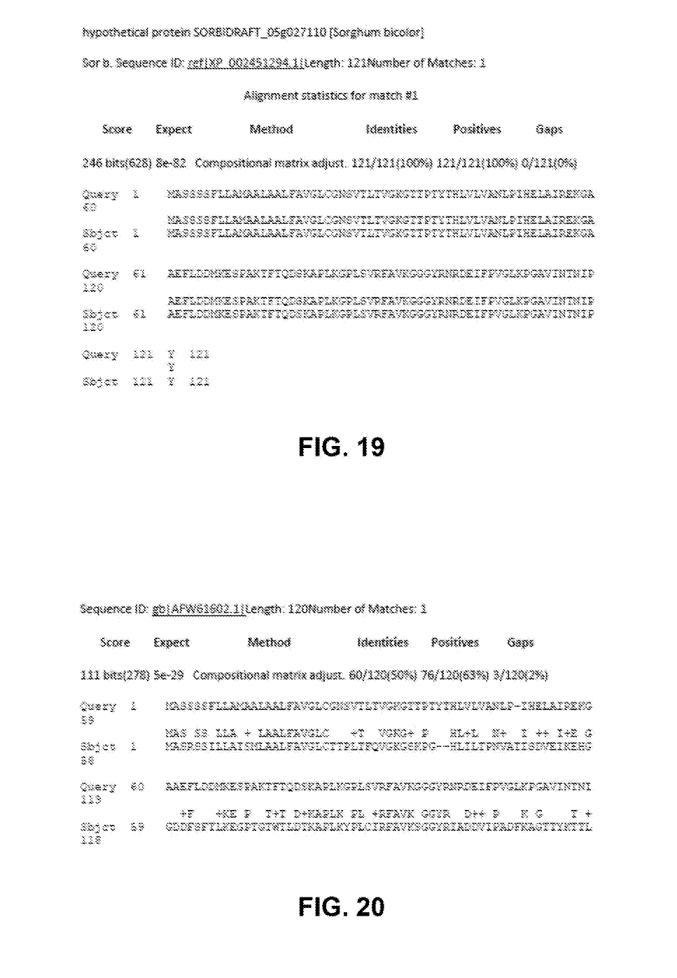

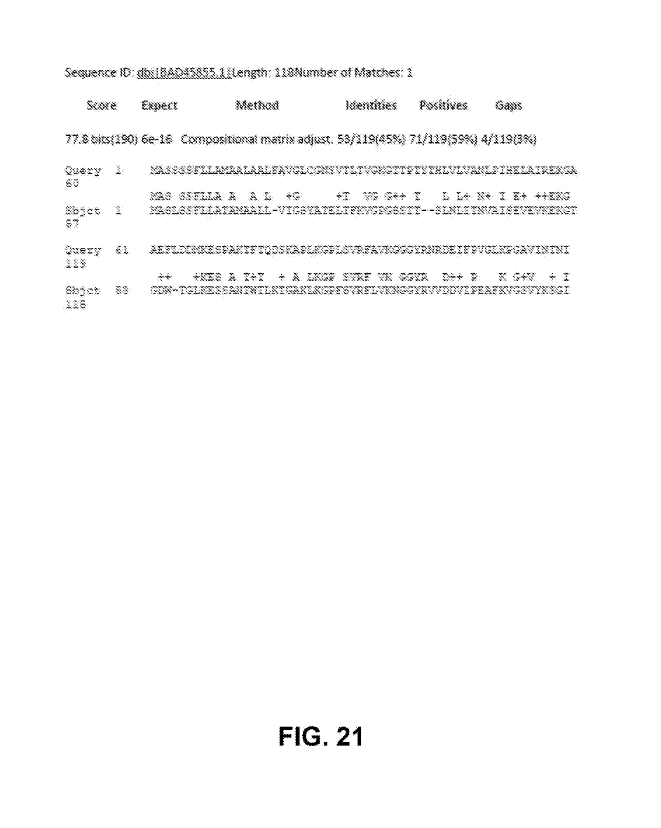

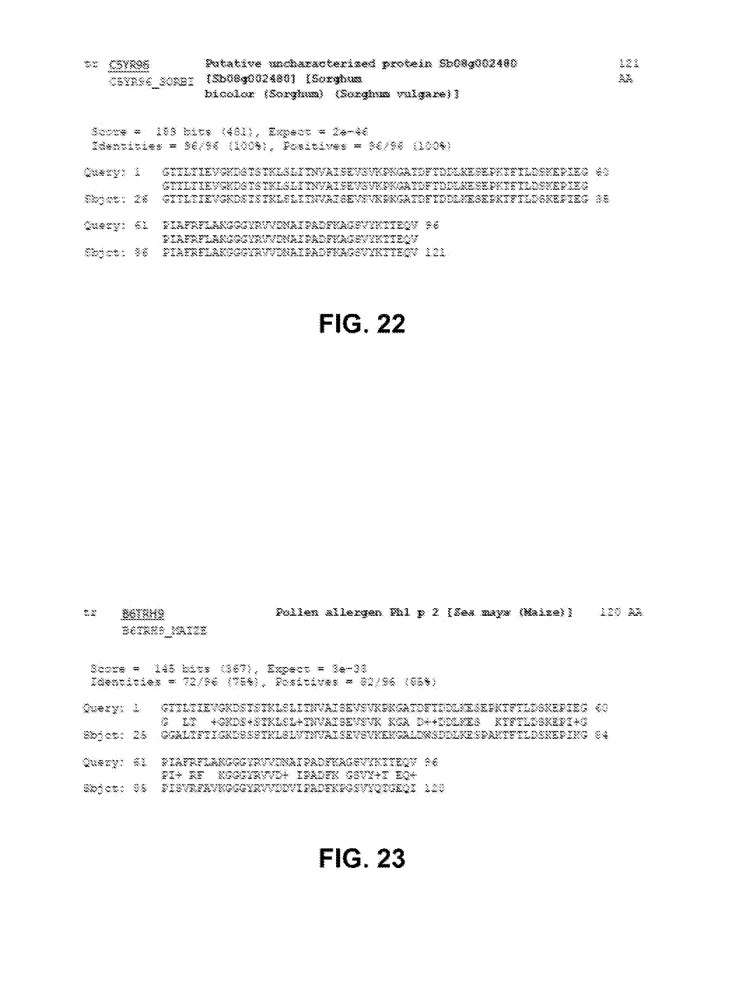

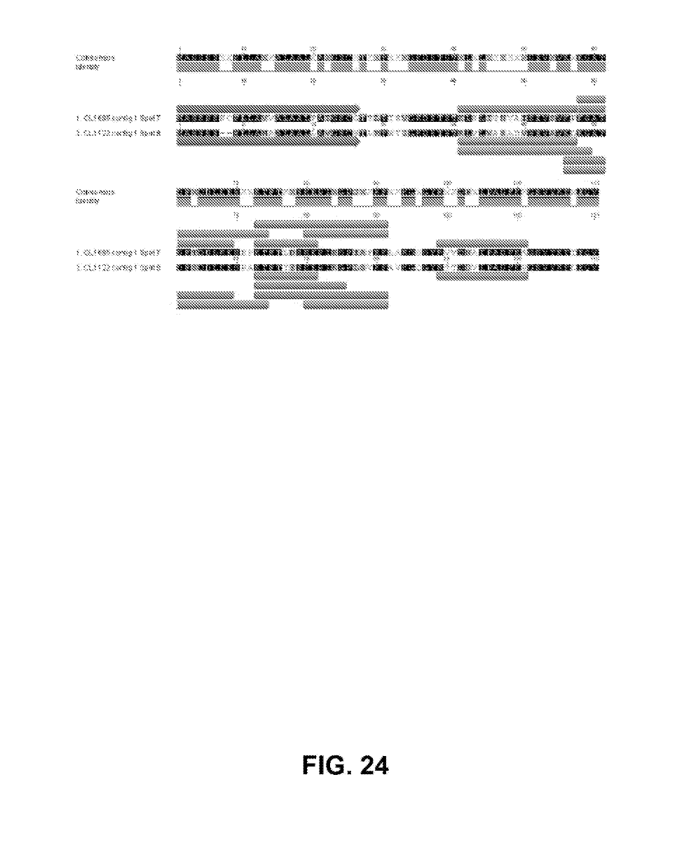

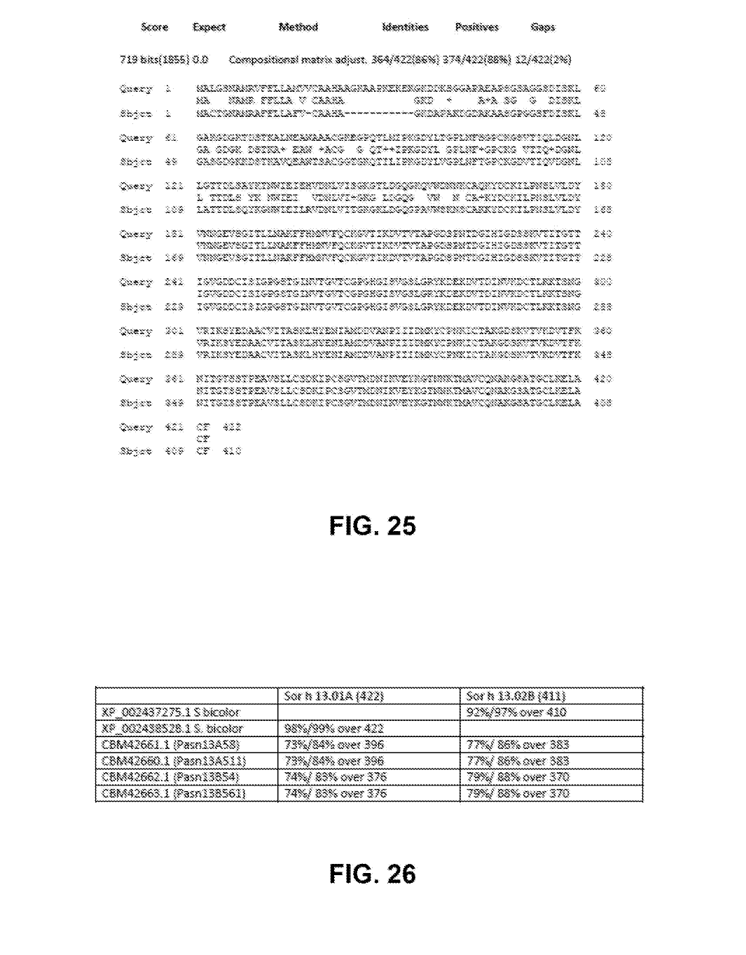

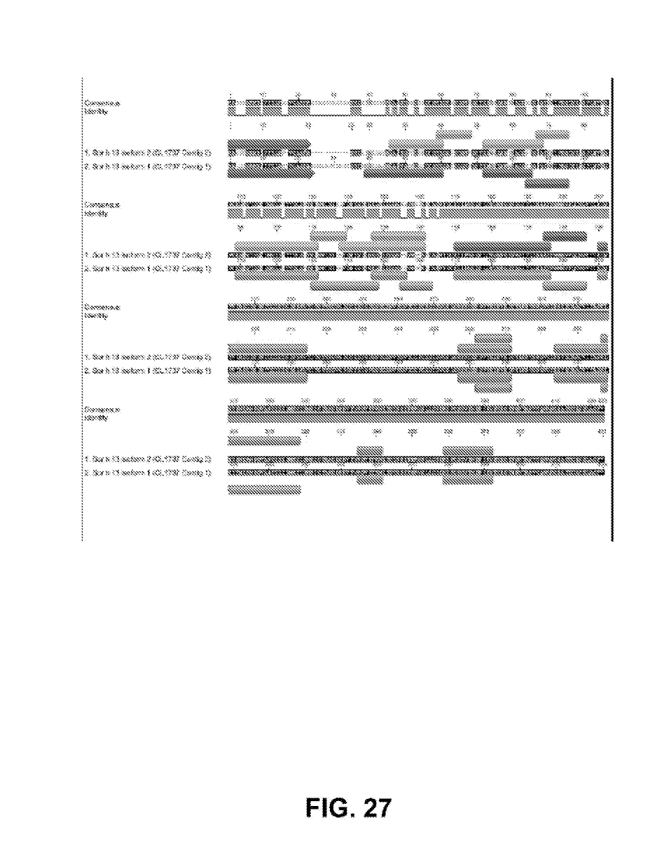

Two IgE reactive proteins of 28 kDa with pI of 6.9 (spot 4) and 5.7 (spot 5) respectively, were observed. The contig with the highest number of unique peptide spectra for spot 4, and second highest for spot 5 was CL2015.1; peptides matching spot 4 and 5 covered 66% and 73% respectively, of the predicted peptide sequence of this contig. A BLAST search revealed that this contig had 100% identity with an hypothetical protein of the related S. bicolor and showed 39% amino acid identity and 59% amino acid similarity with Cyn d 23 (gb AAP80170.1) (FIGS. 13 and 14).

Other putative allergen groups were detected in the total transcriptome and proteome but IgE reactivity was not detected. JGP contained molecules identified as allergens in other sources including reticuline oxidases (Sor h 4), polcalcins (Sor h 7), extensins (Sor h 11), profilins (Sor h 12), Cyn d 15 homologue (Sor b 15) and enolase (Sor h 22) (Table 1).

Sor h 1, 2 3 and 15—β-Expansin Related Proteins.

The P-expansin proteins comprise the group 1 pollen allergen family, yet share sequence similarity with members of the group 2, 3 and 15 allergens as well. The Sor h 1 is a P-expansins, with nucleotide sequence similarity to Phl p 1, of 73%. Further, all cDNA transcripts for Sor h 1 displayed a predicted signal peptide, as well as a putative N-glycosylation site at position 10 characteristic of β-expansins (Table 1, FIG. 7). Typical 3-expansin domains, rare lipoprotein A (Rlp-A-)-like double-psi beta barrel motif etc were predicted.

Within the JGP transcriptome, 13 different cDNA transcripts matching the group 1 allergen family (designated Sor h 1), with representatives matching several different sub-families of f-expansin (Table 1). Of the 13 cDNA transcripts, 2 were recognized as isomers of each other and closely related to the B11 sub-family of expansins. Both isomers had highly abundant transcripts although neither was present in the proteome. In fact, MS data revealed that only 5 cDNA transcripts were actively translated into protein and the transcript abundance ranged from 23172 to 2351 RPKM (Tables 1 and 3).

Within the Sor h 2 allergens, 71 cDNA transcripts were observed, three of which were translated into protein. The group 1 pollen allergen superfamily/α-expansin conserved domain was found. CL1122.1 (Sor h 2.01) and CL1695 (Sor h 2.02) had predicted protein lengths of 119 amino acids (with 23 residue signal peptides) and 121 amino acids (with 25 residue signal peptide) were Sor h 2.01 and Sor h 2.02 showed 61% sequence identity between each other.

Since Sor h 2.03 is so closely related to the Sor h 2 allergen family, it was not possible to identify directly cDNA clones specifically encoding group three allergens.

Since Sor h 2.03 shows substantial homology with pollen expansins, it is conceivable that they are involved in expansin-like activities.

Sor h 4—Reticuline Oxidases.

Related to the FAD/FMN-containing dehydrogenases, 8 cDNA transcripts were identified in JGP and only was detected in the proteome. Demonstrating up to 66% identity with Phl p 4, Unigene 808 matched closely with reticuline oxidase from Zea mays. This putative Sor h 4 and had a gene length of 1913 bp and predicted protein length of 526 amino acids including 22 residue signal peptide. Both the FAD/FMN-containing dehydrogenase and FAD-binding domain were observed. Relative transcript abundance was 1200 RKPM, indicating that this protein is relatively low in frequency (Tables 1 and 3).

Sor h 7—Polcalcins.

cDNA transcripts matching the polcalcin allergen family 7 were abundant in number and transcript of unique cDNA transcripts. Sharing 96% sequence identity with Phl p 7, 68 cDNA transcripts were observed in the JGP transcriptome. Interestingly, 18 cDNA transcripts were identified as belonging to 7 different loci, an example being contig CL216 which had 4 isomers present.

The characteristic EF-hand domain of Sor h7 was observed and the gene ontology (GO) identified amongst several different databases showed that Sor h 7 was a polcalcin with Ca2+-binding capacity. Transcript abundance ranged from 80390 to 41 RSEM-RPKM amongst the cDNA transcripts of which only 2 but few were shown to be translated into protein. Notably, several cDNA transcripts with high RPKM roads e.g. CL637. Contig1 with 80,390.46 was not expressed in the proteome.

Bet v 6—Soflavone Reductase Homolog.

Two cDNA transcripts CL2295 and Unigene 7449 from JGP were shown to match the minor Birch pollen allergen Bet v 6. CL2295 with a predicted size of 309 amino acids showed 66% sequence identity with Bet v 6 (gb AAG22740.1). GO annotation matched that of an isoflavone reductase, a class of proteins believed to be involved in plant defence. The relative transcript abundance was quite low at 763 RPKM Unigene and only three unique spectra were detected in the proteome. (Table 1).

Sor h 11—Ertensins.

There were 14 unique cDNA transcripts identified which had a close match to either the major pollen allergen Lol p 11 or Phl p 11. Unigene 540 matched the sequence of Lol p 11 and Ph p 11 at 87% and 96% identity respectively. Both transcripts contained protein motifs in keeping with the trypsin inhibitor-like family. Unlike Phl p 11, allergens associated with Lol p 11 do not have trypsin-inhibitory capability, but are closer in function to proteins called extensins, which are important constituents of primary cell walls and maintain their integrity. For example, transcript CL1754 has its GO biological process listed as glucuronoxylan biosynthetic process highlighting the link to the extensin family of proteins. Transcript abundance varied widely, with the highest amount belonging to contig CL1754 at 499143 RPKM, and ranging to as low as 2 for Unigene 15400. Only, one cDNA transcript was likely to be translated into protein and that was Unigene 540, which had a RPKM amount of 2479. Generally, gene length ranged from 1253 to 205 bp and predicted protein length for transcript Unigene 540 was 144 amino acids (Tables 1 and 3).

Sor h 12—Profilins

Closely related to the known pollen allergen Phl p 12, 16 cDNA transcripts were identified, with close matches to different profilins Transcript abundance ranged from 26487 RPKM to as low as 3. Unigene 308 was also expressed in the proteome. Profilins are believed to regulate the dynamics of the pollen actin cytoskeleton in germinating pollen, and each of the cDNA transcripts had gene ontologies linking them to actin binding and actin cytoskeleton organisation. Unigene 1043 contained a profilin domain, poly-proline binding sites, actin interaction sites and putative PIP2-interaction sites. Gene length ranged from 991 to 276 bp and predicted protein length of Unigene 308 was 131 amino acids (Tables 1 and 3).

Sor k 13—Polygalacturonase.

Approximately 17 cDNA transcripts closely homologous to Phl p 13 (76% identity) appeared frequently in the JGP transcriptome. Similarly, peptides of Sor h 13 within the proteome matched 8 unique cDNA transcripts. These cDNA transcripts matched closely the exopolygalacturonase proteins from Zea mays. Most transcripts had the glycosyl hydrolase family-28 domain commonly found in polygalacturonases. Of the 17 cDNA transcripts, CL248 contig 1 had the highest RPKM value of 272584, while Unigene 17192 had the lowest at 1 (Table 3). Like Sor h 7, Sor h 13 was observed to have several isoforms, with CL986 contig 1 being observed in the proteome, while contig 2 was absent. The other isoforms present belonged to CL1737, with both contigs being expressed in the proteome. ClustalW alignment between the predicted protein of both isoforms of CL1737 from JGP, the actual peptides from MS showed both isoforms are expressed in the proteome, but that the sequence identity is also very high and the pattern of hydrophobic amino acids between each sequence is nearly identical (FIG. 33). Gene length ranged from 2334 down to 203 bp (Tables 1 and 3).

Sor h 22—Enolase

Within the JGP transcriptome, 3 cDNA transcripts closely matched the enolase allergen of Bermuda grass pollen Cyn d 22. Peptides matching cDNA CL70 contigs 1 and 2 were identified in the proteome. (Tables 1 and 3).

Sor h 23-Cyn d 23 Like Protein.

There were 36 cDNA transcripts identified matching the uncharacterised pollen allergen Cyn d 23, 2 of which were isomers of each other. Relatively abundant, 3 of the transcripts including CL2015.1 were detected in the proteome, the highest having an RPKM of 211352. Gene length ranged from 1247 to 428 bp (Tables 1 and 3). The closest allergen match for predicted peptide sequence of CL 2015.1 was 39% amino acid identity and 59% similarity with the Bermuda grass pollen allergen Cyn d 23 justifying its designation as a group 23 allergen (FIG. 14). However, there was a domain with high similarity to a domain of the temperate Pooideae group 5 allergens (FIG. 39), indicating this Johnson grass pollen allergen could share allergen properties with the temperate grass pollen group 5 allergen family.

### Discussion

Integrating modern transcriptomic sequencing technology with advanced proteomic and serological analysis has allowed a comprehensive analysis of mature Johnson grass pollen allergen diversity. Knowledge of allergenic components of subtropical grass pollens will facilitate increased understanding of the contribution to the disease burden of allergic rhinitis in subtropical regions of the world. It was revealed that Sor h 1 is a major allergen of JGP. New isoforms including one with a basic pI were discovered all displaying IgE reactivity with relevant patient sera and mAb to group 1 allergens. Our data suggests Sor h 1 may have utility for more sensitive diagnosis of JGP allergy than whole JGP extract.

Sor h 1 displayed five allergen spots and only two gene loci, indicative of post translational modifications. That related contigs CL153.1 and CL153.2 encoding Sor h 1 only differ in their respective signal peptide, suggests alternative splicing may regulate intracellular location. This phenomenon was noticed in Sor h 2 and 13 as well. Differences between basic and neutral isoforms of Sor h 1 may be relevant for the allergenic activity and epitope recognition at both a T and B cell level (Chabre et al. Clin Exp Allergy, 2010).

Sor h 1 and 2 appear to be homologues of the β-expansin family, cell wall loosening enzymes found in the cell walls of most plant tissues (Cosgrove et al., Proc Natl Acad Sci USA, 1997). Sor h 2 isoforms are clearly related to the C-terminal domain of Sor h 1 but still separate out into their own clade, which corresponds with literature on Phl p 2 and 3 and Lol p 2 and 3 (Peterson et al., Proteomics, 2006; Sidoli et al, J Biol Chem, 1993; Tamborini et al., Mol Immunol, 1995).

The newly identified allergen designated as Sor h 13, was the second most IgE reactive allergen of JGP. However, its frequency of IgE reactivity did not achieve the 50% mark of a major allergen in this cohort of patients and the level of IgE reactivity was significantly lower than JGP or Sor h 1. Polygalacturonase allergens are located in the internal cell wall and cytoplasm of mature pollen grains (Grote et al., Int Arch Allergy Immunol, 2005) and have previously been shown to accumulate in mature barley pollen (Pulido et al., Plant Cell Rep, 2009). The relatively high transcript copies in JGP for both Sor h 13 isoforms (123,023 and 82,537 for contig CL1737.1 and CL1737.2 respectively) suggests a similar pattern of development. Polygalacturonase arise from a large gene family that serve various functions (Kim et al., Genome Biol, 2006). It is hypothesised that these enzymes supply wall precursors for pollen tube growth, as well as assist the penetration of the pollen tube into the stigma and style tissues via degradation of their cell walls (Chiang et al., Plant Physiol Biochem, 2006; Niogret et al., Plant Mol Biol, 1991).

An IgE reactive protein designated Sor h 23, showed sequence homology to Cyn d and Ory s 23 (Russel et al., Mol Plant, 2008; <http://www.allergome.org/script/dettaglio.php?id_molecule=691>). The allergenic significance of this group 23 allergen is yet to be further characterised, but its relative transcript abundance in JGP (≈211,351 copies), suggests it has a necessary function within the mature pollen. Although a second contig with 67.6% identity to CL2015.1 was present in the JGP transcriptome, the observed peptide spectra of IgE reactive spots 4 and 5 only matched CL2015.1. The alignment between both these related contigs indicated that the second sequence is more consistent with an orthologous gene from a different locus, which fits with the polyploidy nature of the S. halepense genome.

That IgE reactivity to berbine bridge oxidase, profilin, polcalcin or enolase proteins, corresponding to putative allergens Sor h 4, Sor h 12, Sor h 7, and Sor h 22 respectively, was not detected may be an issue of assay sensitivity or size of the study population. Proteome and transcriptome analysis confirmed the presence of each of these potential allergens within JGP, but the data did not allow for discernment of the abundance of their expression. In patients from Europe, primarily exposed to temperate grass pollens, most of these proteins are minor allergens. Grass pollen allergic patients show low frequency of serum IgE reactivity with the Timothy grass pollen allergens (Phl p 12) and polcalcin (Phl p 7) of 24% and 7% respectively whereas the frequency of IgE reactivity with Phl p 4 is high at 85% (Westritschnig et al., Eur J Clin Invest, 2008). Of 10 sera from Taiwanese patients with Bermuda grass pollen allergy, Kao observed IgE reactivity with enolase (Cyn d 22) in all ten, BG60 (Cyn d 4) in five, profilin (Cyn d 12) in two but none with polcalcin (Cyn d 7). Others report IgE reactivity with a lambda phage clone expressing Cyn d 7 in three of 30 subjects with Bermuda grass pollen allergy (Smith et al., Int Arch Allergy Immunol, 1997). The importance of these potential proteins as allergens of JGP needs to be determined using purified or recombinant protein preparations for testing in larger populations from other subtropical regions where JGP is an important pollen allergen source eg South Africa, Thailand and India.

Whilst incidences of AR and asthma have plateaued in developed nations, the frequency of allergic respiratory diseases shows greater variability and immense impact in countries with emerging economies (The Global Asthma Report 2011, International Union Against Tuberculosis and Lung Disease: Paris, France). Knowledge of sensitization to subtropical grass pollen allergens will assist with development of clinical guidelines for appropriate grass pollen allergy diagnosis and immunotherapy in places where people are predominantly exposed to subtropical grass pollens. The newly identified molecular allergenic components of JGP identified here will have global utility to customise diagnosis and treatment for subtropical grass pollen allergy. Integration of the total transcriptome, proteome and allergome of a clinically significant allergen has not previously been reported. This combined molecular and bioinformatics approach is amenable for use in discovery of unknown allergenic components of diverse sources for which the genome has not been determined.

TABLE 1
Putative grass pollen allergen encoding transcripts identified within the
transcriptome of Johnson grass pollen.

| | | | | Predicted | | | | |
|---|---|---|---|---|---|---|---|---|
| | [symbol] | | Predicted | SP/ | Predicted | | | InterPro |
| Allergen | bits in | Transcript | pI/MW | mature | N- | | | family |
| group | transcriptome | ID* | (kDa) [symbol] | peptide [symbol] | Glycosylation [symbol] | Annotaion | Comments [symbol] | identified [symbol] |
| 1 | 51 | CL153.1 | 7.04/25.8 | 27/239 | Asn10 | β-Expansin | Predicted peptide for | IPR005795, |
| | | | | | | | CL153.1 and UG493-492 | IPR007112, |
| | | | | | | | show 57% identity and | IPR007117, |
| | | | | | | | 73% similarity | IPR007118 |
| | | CL153.2 | 7.04/25.8 | 27/239 | Asn10 | | | |
| | | UG 493-492 | 9.13/26.5 | 24/137 | Asn10 | | | |
| 2 and 3 | 71 | CL1122.1 | 6.29/10.4 | 23/96 | no Asn | Expansin C- | Predicted peptide identity | IPR007117 |
| | | CL1121.2 | 9.35/10.5 | 23/98 | none | terminal fragment | between CL1122.1 and | |
| | | CL2095.1 | 5.02/10.2 | 25/96 | none | | CL1122.2 38% | |
| | | | | | | | CL1122.1 and CL1695, | |
| | | | | | | | 65%, CL1122.2 and | |
| | | | | | | | CL1695, 48% | |
| 4 | 8 | UG 808 | 9.35/55.5 | 22/504 | Asn335 | FAD linked | | IPR006094, |
| | | | | | | oxidase | | IPR012951, |
| | | | | | | Berberine-like | | IPR016166, |
| | | | | | | | | IPR016167, |
| | | | | | | | | IPR016169 |
| 7 | 67 | UG 461 | 4.72/8.9 | 0/96 | no Asn | EF hand-like | | IPR011992, |
| | | UG 293 | | | | calcium binding | | IPR002048, |
| | | | | | | protein | | IPR018247 |
| 11 | 14 | UG 540 | 5.09/13.6 | 19/125 | Asn30 | extensin family | 100% ID to S. bicolor | IPR006041 |
| | | | | | | | ref XP_002440811.1 | |
| 12 | 15 | UG 308 | 4.91/14.3 | no Asn | —/13.1 | Profilm, actin | | IPR005455, |
| | | | | | | binding protein | | IPR027310 |
| 13 | 17 | CL1737.1 | 6.39/41.6 | Asn80, | 23/399 | Polygalacturonase | | IPR000743, |
| | | | | 235 and | | Glycoside | | IPR006620, |
| | | | | 376 | | hydrolase family | | IPR011050, |
| | | | | | | 28 | | IPR012334 |
| | | CL1737.2 | 7.84/40.5 | Asn69, | 22/388 | | | |
| | | | | 224, 365 | | | | |
| 22 | 5 | CL70/1 | 4.94/48.1 | Asn19, | —/446 | [symbol] | 89% indentity and 95% | IPR000941, |
| | | | | 146 and | | | similar to Cynd 22 | IPR020809, |
| | | | | 335 | | | | IPR020810, |
| | | | | | | | | IPR020811 |
| 23 | 36 | CL2015.1 | 6.22/22.2 | none | 17/211 | 39% identical and | 100% identical to | unresolved |
| | | | | | | 59% similar to | hypothetical protein of S. bicolor | |
| | | | | | | predicted protein | XP_002446575.1 | |
| | | | | | | Cyn d 23 gb | | |
| | | | | | | AAP80170.1 | | |

[symbol]

indicates data missing or illegible when filed

TABLE 2
Characteristics of observed IgE reactive molecular allergen components of
JGP.
TABLE 2 Characteristics of observed IgE reactive molecular allergen components of JGP

| | | Number of | | | | | | |
|---|---|---|---|---|---|---|---|---|
| | Observed | spectral | | Predicted | | | Peptide | |
| | pI/MW | Unique | JGP | pI/MW | Predicted N- | Allergen | Coverage | Closest match identified by NBCI non r [symbol] |
| Spot | (kDa) | peptides | transcript | (kDa) | glycosylation | designation | (%) | protein BLAST search |
| 1 | 6.8/30 | 63/16 | CL153 | 7.04/25.8 | Asn10 | Sor h 1.01A | 78.2 | 100% identity to S. bicolor XP_002466021.1 |
| 2 | 71/30 | 64/14 | CL153 | 7.04/25.8 | Asn10 | Sor h 1.01A | 78.2 | |
| 3 | 10.5/30 | 11/5 | UG | 9.13/26.5 | Asn10 | Sor h 1.02B | 76.1 | 99% identity to S. bicolor XP_002467539 |
| | | | 493/492 | | | | | |
| 4 | 5.7/29 | 43/14 | CL2015 | 6.22/22.2 | none | Sor h 23 | 66.2 | 100% identity to S. bicolor XP_002446575.1 |
| 5 | 6.9/29 | 39/12 | CL962 | 6.85/23.8 | none | ND | 73 | 76% identity to sequence of Z. mays |
| | | | | | | | | NP_001131253.1 |
| | | 39/11 | CL2015 | 6.22/22.2 | none | Sor h 23 | | |
| | | 41/40 | CL994 | 5.97/20.4 | Asn50, 79 | ND | | 32% indentity to S. bicolor XP_002437741.1 |
| 6 | 5.0/25 | 17/7 | UG358 | 3.06/19.0 | Asn 53/129 | ND | 56.8 | Predicted SP of 32 AA with mature peptide |
| | | | | | | | | of 175 AA. 100% identical to S. bicolor |
| | | | | | | | | XM_002448839.1 UniProKB/ |
| | | | | | | | | UniProtSb03e0009101 with pectin |
| | | | | | | | | methylesterase incubation domain |
| 7 | 4.9/12 | 57/11 | CL1595 [symbol] | 5.07/10.2 | none | Sor h 2.01 | 48.8 | 100% identity to S. bicolor C5YR96 |
| | | | | | | | | (UniProtKB/UniProt Sb [symbol] g002480) and |
| | | | | | | | | 75% identity to Z. mays |
| | | | | | | | | homologue of Phi p 2 B6TRH9 |
| | | | | | | | | (UniProtKB) |
| 8 | 5.9/12 | 57/11 | CL1121.1 | 6.19/10.4 | none | Sor h 2.01 | 67.4 | 75% identity to Z. mays homologue of |
| | | | | | | | | Phl p 2 HSTRH9 |
| 8A | 10/16 | | CL1122.2 | 9.35/10.5 | none | Sor h 3 | | no proteomic data obtained |
| 9A | 5.7/55 | 568/30 [symbol] | CL1737.1 | 6.59/41.6 | Asn80, 235 | Sor h 13.01A | | 98% identity to S. bicolor XP_002438528.1 |
| | | | | | and 376 | | | |
| 9B | 5.9/55 | | | | | Sor h 13.01A | 58.8 | |
| 9C | 6.6/55 | | | | | Sor h 13.01A | | |
| 10A | 7.0/54 | 58/28 [symbol] | CL11737.2 | 7.84/40.5 | Asn69, 224, | Sor h 13.01B | 58.3 | 92% identity to S. bicolor XP_002437273.1 |
| | | | | | 365 | | | |
| 10C | 7.6/54 | | | | | Sor h 13.01B | | |

[symbol]

Mass spectrometry was performed on purified group 13 allergen [symbol]
Identity between CL1122.1 and CL1122.2, CL1122.1 and CL1695, and CL1122.2 and CL1695, was 58%, 65% and 48% respectively. pI of CL1122.2 consistent with group 3 allergen. [symbol]
Sequence identity code for NBCI database unless [symbol] indicated. ND, not determined Asn. A [symbol] [symbol]
indicates data missing or illegible when filed

TABLE 3
Representation of allergen transcripts and proteins in the total Johnson grass
pollen allergome.

| | | No. of cDNA | Transcript | Number of peptide | | |
|---|---|---|---|---|---|---|
| | Allergen | Clones Present in | Abundance | spectra in the | Percentage identity | |
| Family | type | protosome | (RSEM-RPCAM) | proteosome | with known allergen | Domain/motif |
| β-Expansins | Group 1 | 13 hits, including | 33573.73 | 71 | 73% with Phi p 1 | Rare lipoprotein A |
| | | B11, A3Z, A26, 89 | to 5 | | | (RipA)-like double-psi |
| | | B1, B4, B7 | | | | beta-barrel; |
| | | | | | | Group 1 pollen allergen. |
| C terminal of | Group 2 | 7 hits, several | 33573.73 | 14 | 32% with Phi p 2 | Group 1 pollen allergen |
| expansin | | matches | to 5 | | | superfamily/α-expansin |
| Expansin related; | Group 3 | 7 hits, several | 33573.73 | | | Pollen allergen/expansin |
| similiar to Group 2 | | matches | to | | | C terminal domain |
| allergens | | | 2259.2 | | | |
| [symbol] oxidases | Group 4 | 1 hit | 1300 | | 95% with Phi p 4 | PAD/ [symbol] -containing |
| | | | | | | dehydrogenase, |
| | | | | | | PAD-binding domain. |
| Polcalcine | Group 7 | 42 hits, very | 234808.83 | | 70% with Phi p 7 | Ca²⁺-binding protein (EF- |
| | | abundant | to 5 | | | Hand superfamily) |
| Isoflavone | Set v 6 | 2 hits, very rare | 763.01 | 5 | | Oxidoreductase activity |
| reductase homolog | | | to | | | |
| | | | 38 | | | |
| Pectin esterase | Set v [symbol] | | | 8 | | |
| Extensin | Group 11 | 9 hits, several | 499142.69 | 22 | | Pollen Ole e 1 |
| | | matches | to 2 | | | allergen/extension |
| | | | | | | domain |
| Profilin | Group 12 | 4 hits, including | 26486.93 | 62 | | Profilin |
| | | prf A, prf 1, prf 2 | to 3 | | | Poly-proline binding |
| | | | | | | sites, |
| | | | | | | Actin interaction sites, |
| | | | | | | PIP2-interaction sites. |
| Polygalacturonase | Group 13 | 12 hits, very | 272583.93 | 75 | 77% to Phi p 13 | Glycosyl hydrolase |
| | | abudant | to 1 | | | family. |
| C terminal of | Cyn d 15 | Similar to | | | | |
| expansin | | transcripts | | | | |
| | | matching | | | | |
| | | Phi p 1 | | | | |
| Enoiase | Group 22 | 3 hits, very rate | 728.86 | 64 | | Glycolysis, |
| | | | to 0 | | | phosphopyruvate |
| | | | | | | hydratase activity |
| Pollen allergen like | Group 23 | 7 hits, very | 211351.24 | | 39% to Cyn d 23 | Undetermined function |
| Cyn d 23 | | abudant | to | | | |
| | | | 22.05 | | | |

[symbol]

indicates data missing or illegible when filed

TABLE 4
Putative allergen components of JGP; matches between JGP transcripts
expressed in JGP proteome and grass pollen allergen sequences in Allergome
database

| Allergen | | | | | | |
|---|---|---|---|---|---|---|
| family | JGP Transcripts | Protein family | Chloridoideae | Panicoideae | Ehrhartoideae | Pooideae |
| 1 | CL153.1, UG 493, | beta-expansin | 47 | 57 | 53 | 169 |
| | UG492 UG335, UG551 | | | | | |
| 2/3 | CL1122.1, CL1122.2, | C terminal beta-expansin | 5 | 66 | 164 | 117 |
| | CL1695.1, UG 8760 | | | | | |
| 4 | UG808 | Reticuline oxidase-like | 1 | 1 | 0 | 17 |
| | | protein | | | | |
| 5/6 | UG397, UG1403, | Ribonuclease | | | | 7 |
| | CL2015.1, CL962.1 | | | | | |
| 7 | CL1715.1, UG451, | Polcalcin | 22 | 15 | 7 | 28 |
| | UG681, UG832 | | | | | |
| 11 | CL1754.1, CL2052.1, | Glucuronosyl-transferase | 0 | 16 | 4 | 16 |
| | UG540, UG578, | | | | | |
| 12 | UG1043, UG308, | Profilin-A | 2 | 53 | 7 | 57 |
| | UG342 | | | | | |
| 13 | CL1737.1, CL1737.2, | Exopolygalact-uronase | 2 | 53 | 7 | 57 |
| | CL248, CL986.1, | | | | | |
| | UG1334, UG332, | | | | | |
| | UG552, CL110 | | | | | |
| 15^{a} | UG551, CL153.1, | Expansin like protein | 6 | 0 | 0 | 0 |
| | UG492, CL1122.1, | | | | | |
| | CL1122.2, CL1695.1 | | | | | |
| 22 | CL70.1 | Enolase 1 | 1 | 1 | 0 | 0 |
| 23 | UG397, CL962.1, | Unknown | 5 | 0 | 6 | 0 |
| | CL2015.1, CL2015.2, | | | | | |
| | CL1403 | | | | | |
| 24 | CL830.1 | Pathogenesis-related protein | 1 | 0 | 0 | 0 |
| | | 1A | | | | |
| 25 | CL1152.1, UG2745, | Thioredoxin H-type | 0 | 11 | 0 | 11 |
| | UG5446, UG6038, | | | | | |
| | UG6635, UG7876 | | | | | |
| 12pT | CL1444.1, CL200.1 | Beta-fructofuran-osidase | 0 | 0 | 0 | 4 |
| Total | | | 90 | 404 | 255 | 453 |
| matches: | | | | | | |

Matches between peptides predicted by translation of JGP transcriptome in all 6 reading frames and allergen sequences in Allergome.org. Matches were filtered for those JGP transcripts expressed in the proteome, with query length Over 50 amino acids, percentage identity of 30% or greater (identities/(align length-gap length)) and alignment over 30% of the query length ((align length-gap length)/query length). The number of matches with grass pollen allergens^{42,43,58} (after exclusion of allergens restricted to seed or other tissue) within each subfamily and the corresponding JGP transcripts are given. Those matches specific to subtropical grasses are in bold.
^{a}Matches to Cyn d 15 also show homology with group 1 and 2.

### REFERENCES

Avliogln A et al Cloning and characterization of the major allergen of Sorgham halepense, a subtropical grass. Molecular Biology and Immunology of Allergens, ed. D. Kraft and A. Schon. 1993, CRC Press: Boca Raton. 161-164.
Bauehau V, Durham S R. Prevalence and rate of diagnosis of allergic rhinitis in Europe. Eur Respir J 2004, 24:758-764.
Beggs P J, Bennett C M. Climate change, aeroallergens, natural particulates, and human health in Australia: state of the science and policy. Asia Pac J Public Health 2011; 23: 546-53.
Brozek J L, Bousquet J, Baena-Cagnani C E, Bonini S, Canonica G W, Casale T B et al. Allergic rhinitis and its impact on asthma (ARIA) guidelines: 2010 revision. J Allergy Clin Immunol 2010; 126:466-476.
Bousquet J, Bodez T, Gehano P, Klossek J M, Liard F, Neukirch F, et al. Implementation of guidelines for allergic rhinitis in specialist practices. A randomized pragmatic controlled trial. Int Arch Allergy Immunol 2009; 150:75-82.
Bufe A, Eberle P, Franke-Beckmann E, Funck J, Kimmig M, Klimek L, et al. Safety and efficacy in children of an SQ-standardized grass allergen tablet for sublingual immunotherapy. J Allergy Clin Immunol 2009; 123: 167-173, e167.
Chabre H, Gouyou B, Huet A, Boran-Bodo V, Nony E, Hrabina M et al. Molecular variability of group 1 and 5 grass pollen allergens between Pooideae species: implications for immunotherapy. Clin Exp Allergy 2010; 40: 505-19.
Chiang J Y, Bale N, Hsu S W, Yang C Y, Ko C W, Hsu Y F, Swoboda I, Wang C S. A pollen-specific polygalacturonase from lily is related to major grass pollen allergens. Plant Physiol Biochem 2006; 44:743-51.
Cook M, Douglas J A, Mallon D, Mullins R, Smith J, Wong M. The economic impact of allergic disease in Australia: not to be sneezed at. Australia: Report by Access Economics Pty Ltd., 2007:1-111.
Cosgrove D J, Bedinger P, Durachko D M. Group I allergens of grass pollen as cell wall-loosening agents. Proc Natl Acad Sci USA 1997; 94: 6559-64.
Davies J M, Bright M L, Rolland J M, O'Hehir R E. Bahia grass pollen specific IgE is common in seasonal rhinitis patients but has limited cross-reactivity with Ryegrass. Allergy 2005; 60: 251-5.
Davies J M, Mittag D, Dang T D, Symons K, Voskamp A, Rolland J M, et al. Molecular cloning, expression and immunological characterisation of Pas n 1, the major allergen of Bahia grass Paspalum notatum pollen. Mol Immunol 2008; 46: 286-93.
Davies J M, Voskamp A, Dang T D, Pettit B, Loo D, Petersen A, et al. The dominant 55 kDa allergen of the subtropical Bahia grass (Paspalum notatum) pollen is a group 13 pollen allergen, Pas n 13. Mol Immunol 2011; 48: 931-40.
Davies J M, Dang T D, Voskamp A, Drew A C, Biondo M, Phung M, et al. Functional immunoglobulin E cross-reactivity between Pas n 1 of Bahia grass pollen and other group 1 grass pollen allergens. Clin Exp Allergy 2011; 41: 281-291.
Davies J M, Lil H, Green M, Towers M, Upham J W. Subtropical grass pollen allergens are important for allergic respiratory diseases in subtropical regions. Clin Transl Allergy 2012; 2: 4.
Didler A, Mailing H J, Worm M, Horak F, Jager S, Montagut A, et al. Optimal dose, efficacy, and safety of once-daily sublingual immunotherapy with a 5-grass pollen tablet for seasonal allergic rhinitis. J Allergy Clin Immunol 2007; 120: 1338-1345.
Drew A, Davies J M, Dang T D, Rolland J M, O'Hehir R E. Purification of the major group 1 allergen from Bahia grass pollen, Pas n 1. Int Arch Allergy Immunol 2011; 54: 295-298.
Erbas B, Chang J H, Dharmage S, Ong E K, Hyndman R, Newbigin E, Abramson M. Do levels of airborne grass pollen influence asthma hospital admissions?Clin Exp Allergy 2007; 37: 1641-7.
Grabherr M G, Haas B J, Yassour M, Levin J Z, Thompson D A, Amit I, et al. Full-length transcriptome assembly from RNA-Seq data without a reference genome. Nat Biotechnol 2011; 29: 644-U130.
Grote M, Swoboda I, Valenta R, Reichelt R. Group 13 allergens as environmental and immunological markers for grass pollen allergy, studies by immunogold field emission scanning and transmission electron microscopy. Int Arch Allergy Immunol 2005; 136: 303-10.
Gupta, A. Geoindicators for tropical urbanization. Environmental Geology 2002; 42: 736-42.
Holm L G, Plucknett D L, Pancho J V, Herberger J. The World's Worst Weeds. Honolulu, USA: University Press of Hawaii; 1977. P. 54-61.
Iseli C, Jongeneel C V, Bucher P. ESTScan: a program for detecting, evaluating, and reconstructing potential coding regions in EST sequences. Proc Int Conf Intell Syst Mol Biol 1999; 138-48.
Katelaris C H, Lee B W, Potter P C, Maspero J F, Cingi C, et al. Prevalence and diversity of allergic rhinitis in regions of the world beyond Europe and North America. Clin Exp Allergy 2012; 42: 186-207.
Kim J, Shiu S H, Thoma S, Li W H, Patterson S E. Patterns of expansion and expression divergence in the plant polygalacturonase gene family. Genome Biol 2006; 7: R87.
Li Z W, Trick H N. Rapid method for high-quality RNA isolation from seed endosperm containing high levels of starch. Biotechniques 2005; 38: 872-876.
Li B, Dewey C N. RSEM: accurate transcript quantification from RNA-Seq data with or without a reference genome. BMC Bioinformatics 2011; 12: 323.
Linneberg A, Gislum M, Johansen N, Husemoen L L, Jorgensen T. Temporal trends of aeroallergen sensitization over twenty-five years. Clin Exp Allergy 2007; 37: 1137-42.
McWhorter C G. History, biology, and control of johnsongrass. Reviews of Weed Science 1989; 4: 85-121.
Meltzer E O, Bukstein D A. The economic impact of allergic rhinitis and current guidelines for treatment. Ann Allergy Asthma Immunol 2011; 106:S12-S16.
Morgan J A, LeCain D R, Pendall E, Blumenthal D M, Kimball B A, Carrillo Y, et al. C4 grasses prosper as carbon dioxide eliminates desiccation in warmed semi-arid grassland. Nature 2011; 476: 202-5.
Niogret M F, Dubald M, Mandaron P, Mache R. Characterization of pollen polygalacturonase encoded by several cDNA clones in maize. Plant Mol Biol 1991; 17:1155-64.
Petersen A, Dresselhaus T, Grobe K, Becker W M. Proteome analysis of maize pollen for allergy-relevant components. Proteomics 2006; 6: 6317-6325.
Phillips J W, Bucholtz G A, Fernandez-Caldas E, Bukantz S C, Lockey R F. Bahia grass pollen, a significant aeroallergen: evidence for the lack of clinical cross-reactivity with timothy grass pollen. Ann Allergy 1989; 63: 503-7.
Pulido A, Bakos F, Devic M, Barnaba's B, Olmedilla A. HvPG1 and ECA1: two genes activated transcriptionally in the transition of barley microspores from the gametophytic to the embryogenic pathway. Plant Cell Rep 2009; 28:551-59.
Russel S D, Bhalla P L, Singh M B. Transcriptome-based examination of putative pollen allergens of rice (Oryza sativa ssp. japonica). Mol Plant 2008; 1: 751-59
Seidel D J, Fu Q, Randel W J, Riechler T J. Widening of the tropical belt in a changing climate. Nat Geoscience 2008; 1: 21-4.
Sidoll A, Tamborini E, Gluntini I, Levi S, Volonte G, Paini C et al. Cloning, expression, and immunological characterization of recombinant Lolium perenne allergen Lol p II. J Biol Chem 1993; 268: 21819-25.
Smith P M, Xu H, Swoboda I, Singh M B. Identification of a Ca2+ binding protein as a new Bermuda grass pollen allergen Cyn d 7: IgE cross-reactivity with oilseed rape pollen allergen Bra r 1. Int Arch Allergy Immunol 1997; 114: 265-71.
Tamborini E, Brandauzza A, De Lalla C, Masco G, Siccardi A G, Aroslo P et al. Recombinant allergen Lol p II: expression, purification and characterization. Mol Immunol 1995; 32: 505-13.
Weber R W. Cross-reactivity of pollen allergens: impact on allergen immunotherapy. Ann Allergy Asthma Immunol 2007; 99: 203-11.
Weber R W. Cross-reactivity of pollen allergens: recommendations for immunotherapy vaccines. Curr Opin Allergy Clin Immunol 2005; 5: 563-9.
Westritschnig K, Horak F, Swoboda I, Balk N, Spitzauer S, Kundi M et al. Different allergenic activity of grass pollen allergens revealed by skin testing. Eur J Clin Invest 2008; 38: 260-7.
White J F, Bernstein D I. Key pollen allergens in North America. Ann Allergy Asthma Immunol 2003; 91: 425-435, 435-426, 492.
Ziska L H, Caulfield F A. Rising CO2 and pollen production of common ragweed (Ambrosia artemisifolia), a known allergy-inducing species: implications for public health. Aust J Plant Physiol 2000; 27: 893-98.

1. A method for determining or monitoring sensitivity to a Johnson grass (Sorghum halepense) pollen allergen, or an allergen immunologically cross-reactive with a Johnson grass pollen allergen, in a subject, including the step of determining a presence or absence of an allergen-specific immune response in said subject, wherein the presence of said immune response indicates sensitivity to the Johnson grass pollen allergen or said immunologically cross-reactive allergen, wherein the Johnson grass pollen allergen is or comprises an isolated protein, or a fragment, variant or derivative thereof, the isolated protein comprising an amino acid sequence selected from the group consisting of SEQ ID NOs: 1 to 49.

2. A method for measuring the level of or detecting or monitoring the presence of a Johnson grass pollen allergen, or an allergen immunologically cross-reactive with a Johnson grass pollen allergen, in a sample, including the step of contacting the sample with one or more reagents for a time and under conditions sufficient to detect said Johnson grass pollen allergen or said immunologically cross-reactive allergen, wherein the Johnson grass pollen allergen is or comprises an isolated protein, or a fragment, variant or derivative thereof, the isolated protein comprising an amino acid sequence selected from the group consisting of SEQ ID NOs: 1 to 49.

3. The method of claim 2, wherein the sample is obtained from a mammal.

4. The method of claim 2, wherein the sample is an environmental sample.

5. The method of claim 4, wherein the environmental sample is air or water.

6. The method of claim 2, wherein the sample is, or is derived from, a pharmaceutical composition for immunotherapy.

7. The method of claim 2, wherein the sample is, or is derived from, a diagnostic composition.

8. The method of claim 7, wherein the method is performed to batch standardize the diagnostic composition.

9. The method of claim 2, wherein the sample comprises one or a plurality of other grass pollen-derived allergens in addition to said allergen.

10. The method of claim 2, wherein the method is for determining a relative or absolute amount of the allergen in the sample.

11. The method of claim 2, wherein the reagent is an antibody or antibody fragment.

12. A method of preventing or treating sensitivity to a Johnson grass pollen allergen, or an allergen immunologically cross-reactive with a Johnson grass pollen allergen, in a subject, including the step of administering to said subject a therapeutically effective amount of a Johnson grass pollen allergen or an antibody thereto, or a composition comprising said therapeutically effective amount of a Johnson grass pollen allergen or an antibody thereto, wherein the Johnson grass pollen allergen is or comprises an isolated protein, or a fragment, variant or derivative thereof, the isolated protein comprising an amino acid sequence selected from the group consisting of SEQ ID NOs: 1 to 49.

13. The method of claim 12 further comprising administering one or more additional allergens or one or more antibodies that bind and/or are raised against additional allergens.

14. The method of claim 13 wherein the additional allergens include one or more grass pollen allergens from Bahia grass (Paspalum notatum), Bermuda grass (Cynodon dactylon) and/or Ryegrass (Lolium perenne).

15. The method of claim 12, wherein the therapeutically effective amount of the Johnson grass pollen allergen is administered subcutaneously.

16. The method of claim 12, wherein the therapeutically effective amount of the Johnson grass pollen allergen is administered sublingually.

17. The method of claim 12, wherein the antibody, or a fragment thereof, binds and/or is raised against an isolated protein, or a fragment, variant or derivative thereof, comprising an amino acid sequence selected from the group consisting of SEQ ID NOs: 1 to 49.

18-19. (canceled)

20. A composition comprising one or more of an isolated protein, or a fragment, variant or derivative thereof, comprising an amino acid sequence selected from the group consisting of SEQ ID NOs: 1 to 49 or one or more antibodies or antibody fragments that bind and/or are raised against said isolated protein, fragment, variant or derivative, and one or more pharmaceutically acceptable carriers, diluents or excipients.

21. The composition of claim 20, further comprising one or more additional allergens or one or more antibodies that bind and/or are raised against one or more additional allergens.

22. The composition of claim 21, wherein the additional allergens comprise one or more grass pollen allergens from Bahia grass (Paspalum notatum), Bermuda grass (Cynodon dactylon) and/or Ryegrass (Lolium perenne).

23-25. (canceled)

26. A genetic construct comprising: (i) an isolated nucleic acid molecule comprising a coding nucleotide sequence selected from the group consisting of SEQ ID NOs: 50 to 89, or a fragment, variant or derivative thereof; or (ii) a nucleotide sequence complementary thereto; operably linked or connected to one or more regulatory sequences in an expression vector.

27. A host cell transformed with an isolated nucleic acid molecule comprising a coding nucleotide sequence selected from the group consisting of SEQ ID NOs: 50 to 89, or a fragment, variant or derivative thereof, or the genetic construct of claim 26.

28. A method of producing an isolated protein comprising an amino acid sequence selected from the group consisting of SEQ ID NOs: 1 to 43, or a fragment, variant or derivative thereof, comprising; (i) culturing the previously transformed host cell of claim 27; and (ii) isolating said protein from said host cell cultured in step (i).

29. A diagnostic kit comprising: (i) one or more isolated proteins comprising an amino acid sequence selected from the group consisting of SEQ ID NOs: 1 to 49, or a fragment, variant or derivative thereof; and/or (ii) one or more antibodies, or a fragment thereof, that bind and/or are raised against an isolated protein, or a fragment, variant or derivative thereof, comprising an amino acid sequence selected from the group consisting of SEQ ID NOs: 1 to 49; and (iii) instructions for use.

30. The kit of claim 29, further comprising one or more additional environmental allergens and/or one or more additional antibodies that bind and/or were raised against an environmental allergen.

31. A method of determining the amino acid sequence of one or more grass pollen allergens, including the steps of: (i) preparing cDNA from RNA extracted from a grass pollen; (ii) determining the nucleotide sequence of said cDNA library; (iii) isolating allergenic proteins or fragments thereof from the corresponding grass pollen in (i); and (iv) determining the amino acid sequence of the isolated allergen proteins or fragments thereof from (iii).

32. The method of claim 31, further including the step of confirming the amino acid sequence of (iii) by aligning and comparing the predicted peptide sequence encoding the nucleotide sequence of (ii) with the amino acid sequence of (iii).

33. The method of claim 6, wherein the method is performed to batch standardize the pharmaceutical composition.

34. The method of claim 11, wherein the antibody or antibody fragment binds and/or is raised against an isolated protein, or a fragment, variant or derivative thereof, comprising an amino acid sequence selected from the group consisting of SEQ ID NOs: 1 to 49.
